(19) United States
(12) Patent Application Publication
(10) Pub. No.: US 2025/0079015 A1
(43) Pub. Date: Mar. 6, 2025

(54) DNA METHYLATION BIOMARKERS FOR PRETERM BIRTH

(71) Applicant: WASHINGTON STATE UNIVERSITY, Pullman, WA (US)

(72) Inventor: Michael K. SKINNER, Pullman, WA (US)

(21) Appl. No.: 18/727,885

(22) PCT Filed: Jan. 10, 2023
(86) PCT No.: PCT/US2023/060351

§ 371 Date: Jul. 10, 2024

Related U.S. Application Data
(60) Provisional application No. 63/298,085, filed on Jan. 10, 2022.

(51) Int. Cl. G16H 50/30; C12Q 1/6806; C12Q 1/6809; C12Q 1/6869; G16B 20/20

(52) U.S. Cl. CPC G16H 50/30; C12Q 1/6806; C12Q 1/6809; C12Q 1/6869; G16B 20/20; C12Q 2600/154

(57) ABSTRACT

The present disclosure relates to methods of analyzing the methylation level of differential DNA methylation regions (DMRs) obtained from buccal cells. Also disclosed herein are methods of analyzing methylation level of DMRs for identification and prevention of preterm birth in human subjects.

### CROSS REFERENCE TO RELATED APPLICATIONS

This application claims the benefit under 35 USC § 119 (e) of U.S. Provisional Application Ser. No. 63/298,085, filed on Jan. 10, 2022, the entire disclosure of which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

The present disclosure relates to methods of analyzing the methylation level of differential DNA methylation regions (DMRs) obtained from buccal cells. More particularly, the present disclosure relates to methods of analyzing methylation level of DMRs for identification and prevention of preterm birth in human subjects.

### SUMMARY

Preterm birth (PTB) in humans is generally known as a childbirth that occurs at less than 37 weeks of gestation. Worldwide, preterm birth rates are estimated to be approximately 11%, accounting for about 14.8 million of the live births occurring in 2014. Complications associated with a preterm birth were the leading cause of mortality in children less than five years of age in 2015. Furthermore, children who survive preterm birth are at increased risk of developing future adverse health outcomes, including cognitive disabilities, seizures, visual and hearing impairment, and cardiovascular problems. Although many risk factors associated with preterm birth are known (e.g., genetic variants, exposure to environmental toxicants, presence of multiple fetuses, preeclampsia and ethnicity), more than half of preterm birth cases have an unknown etiology.

A number of potential biomarkers for preterm birth have been identified, for instance alpha-fetoprotein (ms-AFP) and human chorionic gonadotropin (ms-hCG). However, the sensitivity and positive predictive value for these biomarkers are too low for clinically effective screening tests for preterm birth. Moreover, other proposed biomarkers are not useful due to expensive or technically challenging assays or due to poor assay specificity and sensitivity.

Reliable biomarkers for preterm birth could greatly help in predicting which pregnancies are at risk and would improve clinical management and health outcomes for the children. For instance, epigenetics could be explored to identify biomarkers in this regard. Epigenetics is known to include molecular factors and processes around DNA that regulate genome activity, independent of DNA sequence, and that are mitotically stable. Epigenetic factors and procedures include DNA methylation, histone modifications, non-coding RNA, and chromatin structure changes. Assays for DNA methylation can advantageously utilize smaller sample size due to high sensitivity of the assays and are also less expensive and technically demanding than assays for proteins and metabolic products. DNA methylation changes can also be detected in easily obtained surrogate samples (e.g., marker cells not directly associated with the etiology of the pathology), such as cheek buccal epithelial cells. Buccal swabs can provide one of the least invasive and easiest means to obtain a purified cell type via a cheek swab, which includes greater than 90% pure squamous epithelial cells. Any contaminating bacterial molecular data can be removed during the analysis. Since epigenetic differences can be heritable, all somatic cells derived from the embryo of an individual have cell-specific epigenetic changes derived from the germline. Altered DNA methylation sites caused by fetal toxicant exposure, abnormal nutrition, or stress have been found in previous studies to be associated with increased risk of disease in exposed offspring and their descendants (i.e., epigenetic transgenerational inheritance).

Accordingly, the present disclosure provides methods and kits that utilize the concepts of epigenetics to meet the need for biomarkers and diagnostics of preterm birth. Importantly, the methods and kits of the present disclosure can provide options for prevention of preterm birth by identifying susceptible individuals at risk. Furthermore, the methods and kits of the present disclosure can provide important information for administration of preventative therapeutics to be administered to susceptible individuals. For example, such preventative therapeutics could be used to delay or to even prevent the onset of preterm birth.

The methods and kits described herein have several desirable features. First, the methods of the present disclosure can utilize buccal cells that are easily obtained from subjects via a cheek swab and have a high level of purity. Furthermore, although some epigenetic studies have investigated DNA from cells obtained from blood, it is known that blood provides a mixture of cells that contains over 20 different cell types. Since each individual cell type is known to have a unique epigenome to give the cell type its specificity, a mixed cell type analysis can be misleading and reflect changes in cell populations instead of epigenetic change. Accordingly, the present disclosure also contemplates using purified cells to obtain DNA for epigenetic analysis.

Finally, the present disclosure provides the ability to identify preterm birth susceptibility in patients prior to or early in pregnancy. The use of the described preterm birth susceptibility biomarkers permits the initiation of preventative therapeutic intervention to delay or prevent preterm birth in the most susceptible patients. For instance, identification of maternal biomarkers associated with preterm birth could help in the prediction and clinical management of at-risk pregnancies and allow for better preventative care for preterm birth children. Clinical management protocols that could be used to reduce the incidence of preterm birth and infant morbidity include enhanced surveillance of at-risk pregnancies, timely use of prenatal steroids and tocolytics, application of protective uterine monitoring, hospitalization, and operative delivery.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIGS. 1A-1D show principal component analysis (PCA). PCA with sample identification and the African American individuals are circled. FIG. 1A shows mother PCA.

FIG. 1B shows father PCA. FIG. 1C shows female child PCA. FIG. 1D shows male child PCA.

FIGS. 2A-2F show DMR identification and numbers. The number of DMRs were found using different p-value cutoff thresholds. The All-Window column shows all DMRs. The Multiple Window column shows the number of DMRs containing at least two nearby significant windows (1 kb each). The number of DMRs with the number of significant windows (1 kb per window) at a p-value threshold of p<1e-04 for DMR is bolded. FIG. 2A shows mother DMRs. FIG. 2B shows father DMRs. FIG. 2C shows female child DMRs. FIG. 2D shows male child DMRs. FIG. 2E shows mother log-fold-change DMR alterations. FIG. 2F shows father log-fold-change DMR alterations.

FIGS. 3A-3D show DMR methylation log-fold change (X-axis) in comparison with log 2p-value significance (Y-axis). Arrows generally indicate DMR individual's statistical significance (p<1e-04) and non-arrowed places are not statistically significant. FIG. 3A shows mother; FIG. 3B shows father; FIG. 3C shows female child; and FIG. 3D shows male child. The arrows indicate circles in areas demonstrating statistically significant DMRs with log-fold change distribution (i.e., increase or decrease DNA methylation).

FIGS. 4A-4B show DMR group overlaps. FIG. 4A shows DMR p<1e-04 Venn diagram overlap. FIG. 4B shows extended overlaps with p<1e-04 and p<0.05 comparisons. DMR number and percent (%) overlap presented within the rows.

FIG. 5 shows Table 1, which provides a DMR list comprising those identified in buccal samples from the mothers (p<1e-04). The table includes DMR name, chromosomal location (Chr), DMR start nucleotide number for the chromosome (start), length (bp) of the DMR, number of 1 kb significant windows, minimum p-value, CpG number (number per DMR) and CpG density (number per 100 base pair), and gene association within 10 kb and gene functional categories. DMR statistics for EdgeR minimum p-value and reasonable false discovery rate (FDR) minimum p-value are presented. The max LFC (log fold change) is shown, which indicates a log scale that has ratio of perterm/term for methylation levels, (+) increase methylation in preterm or (−) decrease methylation in preterm for the DMR sites. The Accession number is PMID 35232984 and the GEO accession number is GSE GSE194227. In some instances an increase in DNA methylation can comprise hypermethylation of a DMR. In some instances a decrease in DNA methylation can comprise hypomethylation of a DMR.

FIG. 6 shows Table 2, which provides a DMR list comprising those identified in buccal samples from the fathers (p<1e-04). The table includes DMR name, chromosomal location (Chr), DMR start nucleotide number for the chromosome (start), length (bp) of the DMR, number of 1 kb significant windows, minimum p-value, CpG number (number per DMR) and CpG density (number per 100 base pair), and gene association within 10 kb and gene functional categories. DMR statistics for EdgeR minimum p-value and reasonable false discovery rate (FDR) minimum p-value are presented. The max LFC (log fold change) is shown, which indicates a log scale that has ratio of perterm/term for methylation levels, (+) increase methylation in preterm or (−) decrease methylation in preterm for the DMR sites. The Accession number is PMID 35232984 and the GEO accession number is GSE GSE194227. In some instances an increase in DNA methylation can comprise hypermethylation of a DMR. In some instances a decrease in DNA methylation can comprise hypomethylation of a DMR.

FIG. 7 shows Table 3, which provides a DMR list comprising those identified in buccal samples from the female children (p<1e-04). The table includes DMR name, chromosomal location (Chr), DMR start nucleotide number for the chromosome (start), length (bp) of the DMR, number of 1 kb significant windows, minimum p-value, CpG number (number per DMR) and CpG density (number per 100 base pair), and gene association within 10 kb and gene functional categories. DMR statistics for EdgeR minimum p-value and reasonable false discovery rate (FDR) minimum p-value are presented. The max LFC (log fold change) is shown, which indicates a log scale that has ratio of perterm/term for methylation levels, (+) increase methylation in preterm or (−) decrease methylation in preterm for the DMR sites. The Accession number is PMID 35232984 and the GEO accession number is GSE GSE194227. In some instances an increase in DNA methylation can comprise hypermethylation of a DMR. In some instances a decrease in DNA methylation can comprise hypomethylation of a DMR.

FIG. 8 shows Table 4, which provides a DMR list comprising those identified in buccal samples from the male children (p<1e-04). The table includes DMR name, chromosomal location (Chr), DMR start nucleotide number for the chromosome (start), length (bp) of the DMR, number of 1 kb significant windows, minimum p-value, CpG number (number per DMR) and CpG density (number per 100 base pair), and gene association within 10 kb and gene functional categories. DMR statistics for EdgeR minimum p-value and FDR minimum p-value are presented. The max LFC (log fold change) is shown, which indicates a log scale that has ratio of perterm/term for methylation levels, (+) increase methylation in preterm or (−) decrease methylation in preterm for the DMR sites. The Accession number is PMID 35232984 and the GEO accession number is GSE GSE194227. In some instances an increase in DNA methylation can comprise hypermethylation of a DMR. In some instances a decrease in DNA methylation can comprise hypomethylation of a DMR.

FIGS. 9A-9D show DMR chromosomal locations. The DMR locations on the individual chromosomes is represented with an arrowhead and a cluster of DMRs with a black box. All DMRs containing at least one significant window at a p-value threshold of p<1e-04 for DMR are shown. FIG. 9A shows mother DMRs. FIG. 9B shows father DMRs. FIG. 9C shows female child DMRs. FIG. 9D shows male child DMRs. The chromosome number versus size (megabase) is presented.

FIGS. 10A-10H show DMR genomic features. FIG. 10A shows father DMR CpG density. FIG. 10B shows father DMR length. FIG. 10C shows mother DMR CpG density. FIG. 10D shows mother DMR length. FIG. 10E shows male child DMR CpG density. FIG. 10F shows male child DMR length. FIG. 10G shows female child DMR CpG density. FIG. 10H shows female child DMR length.

FIGS. 11A-11B show DMR gene associations. FIG. 11A shows DMR (p<1e-04) associated gene function category frequency. FIG. 11B shows DMR associated gene pathways for mother, father, and female child.

FIGS. 12A-12D show associated gene networks and correlations. FIG. 12A shows mother DMR associated gene correlations. FIG. 12B shows father DMR associated gene correlations. The gene correlations and associated genes are presented for each disease group. FIG. 12C shows female child DMR associated gene correlations. FIG. 12D shows male child DMR associated gene correlations. The gene correlations and associated genes are presented for each disease pathology.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless otherwise indicated, open terms for example “contain,” “containing,” “include,” “including,” and the like mean comprising.

The singular forms “a”, “an”, and “the” can be used herein to include plural references unless the context clearly dictates otherwise. For example, the term “a sample” includes a plurality of samples, including mixtures thereof.

As used herein, the term “about” or “approximately” a number can refer to that number plus or minus 10% of that number. In some cases, about or approximately can refer to that number plus or minus 5% of that number. The term about or approximately a range can refer to that range minus 10% of its lowest value and plus 10% of its greatest value. In some cases, the term about or approximately a range can refer to that range minus 5% of its lowest value and plus 5% of its greatest value. Alternatively, particularly with respect to biological systems or processes, the term can mean within an order of magnitude, within 5-fold, or within 2-fold, of a value. Where particular values or values of a range are described in the application and claims, unless otherwise stated the term “about” meaning within an acceptable error range for the particular value should be assumed.

The term “substantially” or “essentially” can refer to a qualitative condition that exhibits an entire or nearly total range or degree of a feature or characteristic of interest. In some cases, substantially can refer to at least about: 70%, 75%, 80%, 85%, 90%, 91%, 92%, 93%, 94%, 95%, 96%, 97%, 98%, 99% or 100% of the total range or degree of a feature or characteristic of interest.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 5 should be considered to have specifically disclosed subranges such as from 1 to 2, from 1 to 3, from 1 to 4, from 2 to 4, from 3 to 5, etc., as well as individual numbers within that range, for example, 1, 2, 3, 4, and 5. This applies regardless of the breadth of the range.

The terms “determining”, “measuring”, “evaluating”, “assessing,” “assaying,” and “analyzing” are often used interchangeably herein to refer to forms of measurement and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity. The biological entity can be an animal, a plant, or a microorganism. The subject can be tissues, cells and their progeny of a biological entity obtained in vivo or cultured in vitro. The subject can be a mammal. The mammal can be a human. The subject may be a child, an infant, or an adult. In some cases, the human is a male. The subject can be a male human of reproductive age (e.g., older than 10 years of age). In some cases, the subject is female human. In some cases, the female human is of reproductive age. In some cases, a child can be a human of less than or equal to 18 years of age. The subject can be about 1 day old to about 18 years old. In some cases, a subject can be less than 1 day old, for example an infant that has been born. In some cases, a subject can comprise an embryo. In some cases, the subject can be about 1 day old to about 1 year old. In some cases, the subject can be older than 18 years of age. In some cases, the subject can be older than about 10 years, 30 years, 40 years, 50 years, 60 years, 70 year, 80 years or 90 years. The subject can be about 1, 5, 10, 15, 20, 25, 30, 35, 40, 45, 50, 55, 60, 65, 70, 75, 80, 85, 90, 95, 100, 105, 110, 115, 120, 125 or 130 years old. In some cases, the subject can be older than 60 or 65 years of age. The subject may be diagnosed or suspected of being at high risk for a disease. In some cases, the subject may not be necessarily diagnosed or suspected of being at high risk for the disease.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

As used herein, the terms “treatment” or “treating” refers to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of one or more symptoms or of an underlying disorder being treated. For example, a therapeutic benefit can comprise reducing the incidence of preterm birth. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose. Any treatment described herein can be administered in an effective amount or therapeutically effective amount.

As used herein, the term “unit dose” or “dosage form” can be used interchangeably and can be meant to refer to pharmaceutical drug products in the form in which they are marketed for use, with a specific mixture of active ingredients and inactive components or excipients, in a particular configuration, and apportioned into a particular dose to be delivered. The term “unit dose” can also sometimes refer to the particles comprising a pharmaceutical composition or therapy, and to any mixtures involved. Types of unit doses may vary with the route of administration for drug delivery, and the substance(s) being delivered. A solid unit dose can be the solid form of a dose of a chemical compound used as a pharmaceutically acceptable drug or medication intended for administration or consumption. Any treatment described herein can be in a unit dose.

As used herein, “pharmaceutically acceptable salt” can refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness. Any treatment described herein also comprises pharmaceutically acceptable salts of said treatment.

In some cases, a reference level is a reference methylation level. In some cases, a reference level is a control level of methylation. In some cases, a reference level of methylation can be a level of methylation from any of the DMRs in Table 1, Table 2, Table 3, Table 4, or any combination thereof. In some cases, a reference level of methylation can be a level of methylation from a plurality of DMRs in Table 1, Table 2, Table 3, Table 4, or any combination thereof. In some cases, a control level of methylation can be obtained from a diseased individual or a group of diseased individuals such as a group of individuals at risk for preterm birth. In some cases, a control level of methylation can be obtained from a healthy individual or a group of healthy individuals without a disease or condition. In some cases, a control level of methylation can be obtained from a male parent, a female parent, or both that had a term birth. In some cases, a control level of methylation can be obtained from a male child, a female child, or both that had a term birth. In some cases, a control level of methylation is from a known cell type, such as a somatic cell.

As used herein, the term “reference sequence”, can refer to a known nucleotide sequence, e.g. a chromosomal region whose sequence is deposited at NCBI's Genbank database or other databases. A reference sequence can be a wild type sequence.

The term “nucleic acid” and “polynucleotide” can be used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., peptide nucleic acid (PNA)) which can hybridize with naturally occurring nucleic acids in a sequence specific manner analogous to that of two naturally occurring nucleic acids, e.g., can participate in Watson-Crick base pairing interactions. Naturally-occurring nucleotides can include guanine, cytosine, adenine, uracil and thymine (G, C, A, U and T, respectively). In some cases, a nucleic acid can be single stranded. In some cases, a nucleic acid can be double stranded. In some cases, a nucleic acid can comprise a ribonucleic acid (RNA), deoxyribonucleic acid (DNA), or both. In some cases, a polynucleotide may have a modified base.

The section headings used herein are for organizational purposes only and are not to be construed as limiting the subject matter described.

In an illustrative aspect, a first method is provided. The method comprises obtaining a sample of a somatic cell from a human subject; isolating deoxyribonucleic acid (DNA) from the sample, thereby obtaining isolated DNA; detecting and determining a methylation level of a differential DNA methylation region (DMR) comprised in the isolated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and wherein a plurality of distinct DMRs are detected and compared, In an embodiment, the plurality comprises at least 10 distinct DMRs.

In an embodiment, the somatic cell comprises a buccal cell. In an embodiment, the somatic cell consists essentially of a buccal cell. In an embodiment, the somatic cell consists of a buccal cell. In some cases, a cell such as a somatic cell can be an isolated cell. In some cases, a cell such as a somatic cell can be a purified cell. In some cases, an isolated cell can comprise a single cell type. In some cases, a purified cell can comprise a single cell type. In some cases, an isolated cell can substantially comprise a single cell type. In some cases, a purified cell can substantially comprise a single cell type.

In an embodiment, the somatic cell comprises a blood monocyte cell. In an embodiment, the blood monocyte cell is a purified blood monocyte cell. In an embodiment, the somatic cell consists essentially of a blood monocyte cell. In an embodiment, the blood monocyte cell is a purified blood monocyte cell. In an embodiment, the somatic cell consists of a blood monocyte cell. In an embodiment, the blood monocyte cell is a purified blood monocyte cell. Methods for purifying blood monocyte cells are well known in the art to the skilled artisan. In some cases, a cell in a sample can be the same cell type. In some cases, a cell in a sample can be of different cell types. For example, a cell in a sample can be a buccal cell and a blood cell.

In some embodiments, a sample can comprise more than one cell such as a somatic cell. In some cases, a sample can comprise about 10 cells, 100 cells, 1000 cells, 10000 cells, 100,000 cells, 1,000,000 cells, 10,000,000 cells, 100,000,000 cells or more than 100,000,000 cells. In some cases, a sample can comprise about: 1 cell to about 1×10\hat{ }11 cells, 1 cell to about 10 cells, 1 cell to about 100 cells, 10 cells to about 1000 cells, 100 cells to about 10,000 cells, 1000 cells to about 100,000 cells, 10,000 cells to about 1,000,000 cells, 10×10\hat{ }5 cells to about 10×10\hat{ }7 cells, 10×10\hat{ }6 cells to about 10×10\hat{ }8 cells, 10×10\hat{ }7 cells to about 10×10\hat{ }10 cells, or about 10×10\hat{ }8 cells to about 10×10\hat{ }11 cells.

In an embodiment, the somatic cell comprises a muscle cell, a blood cell, a skin cell, a nerve cell, or any combination thereof. In an embodiment, the somatic cell comprises an immune cell. In an embodiment, the somatic cell comprises a muscle cell. In an embodiment, the somatic cell comprises a blood cell. In an embodiment, the somatic cell comprises a skin cell. In an embodiment, the somatic cell comprises a nerve cell.

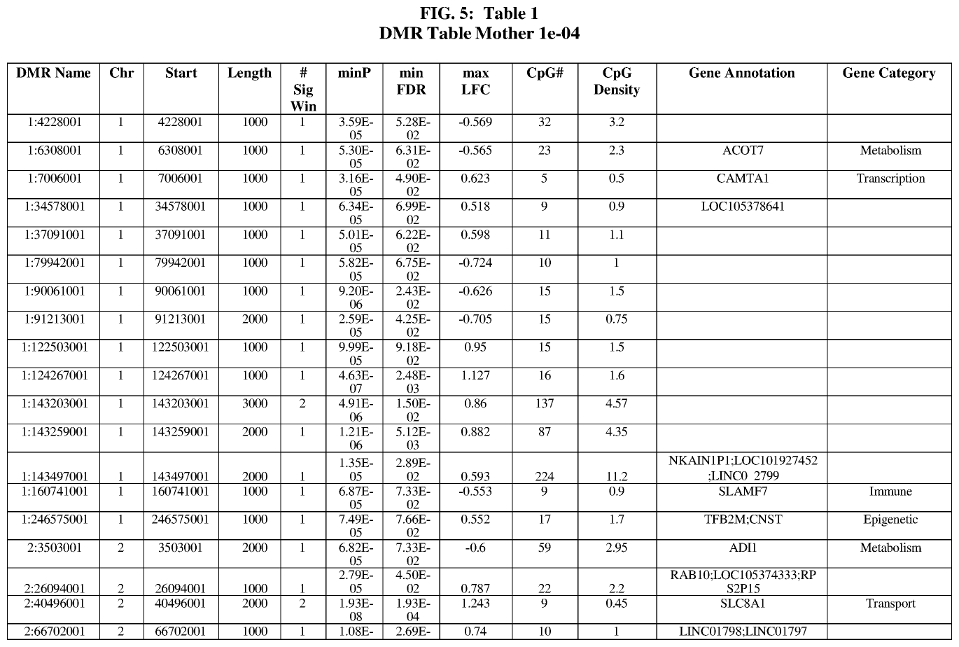

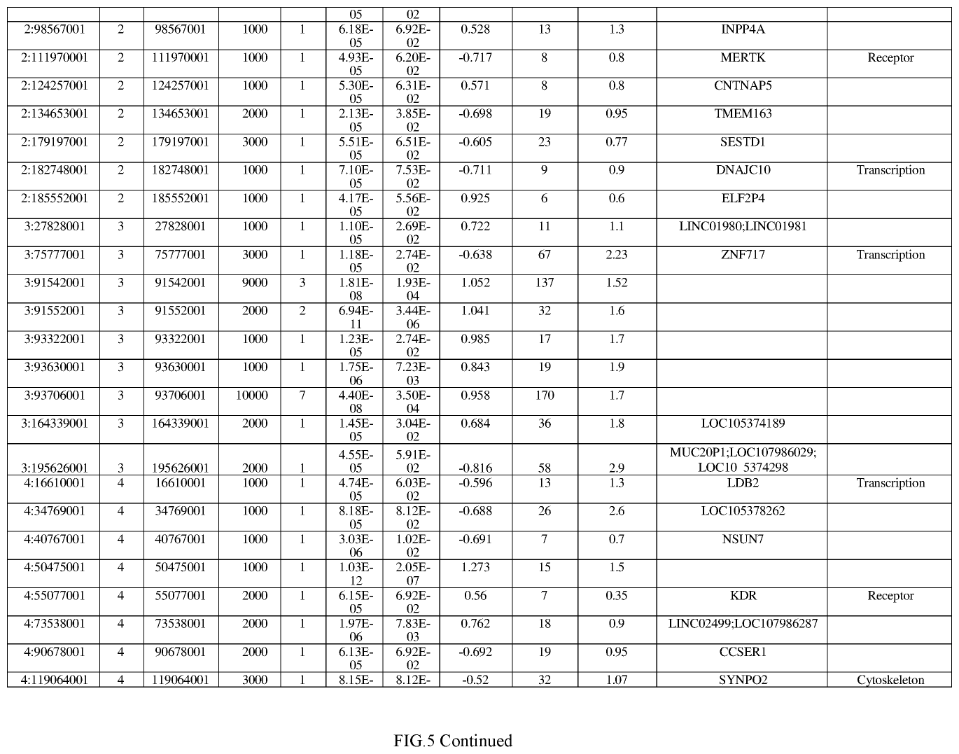

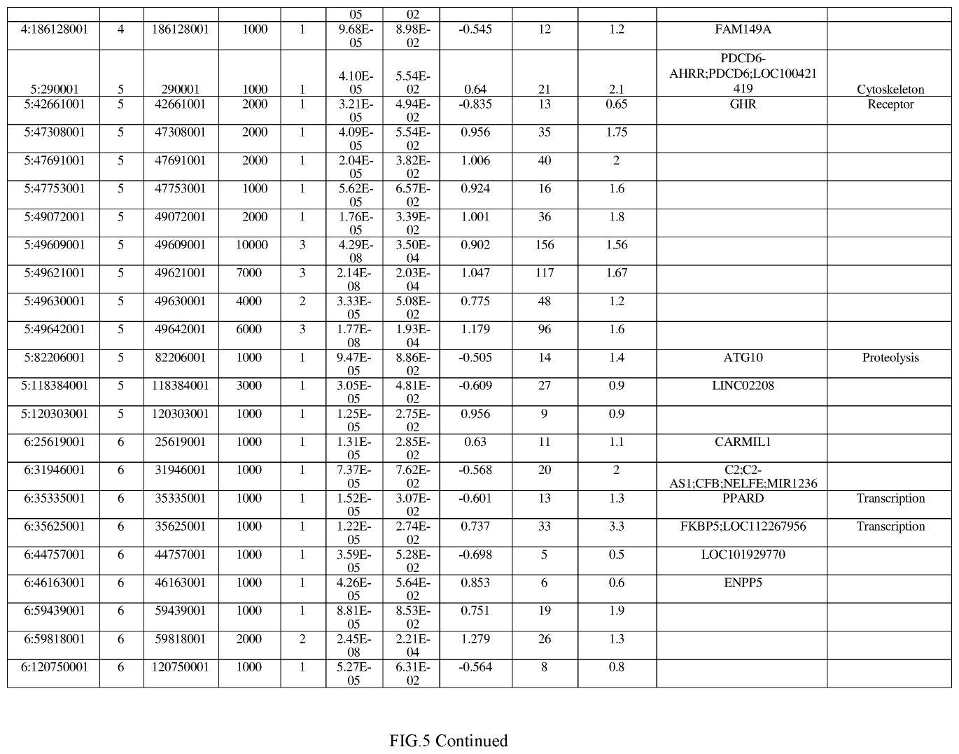

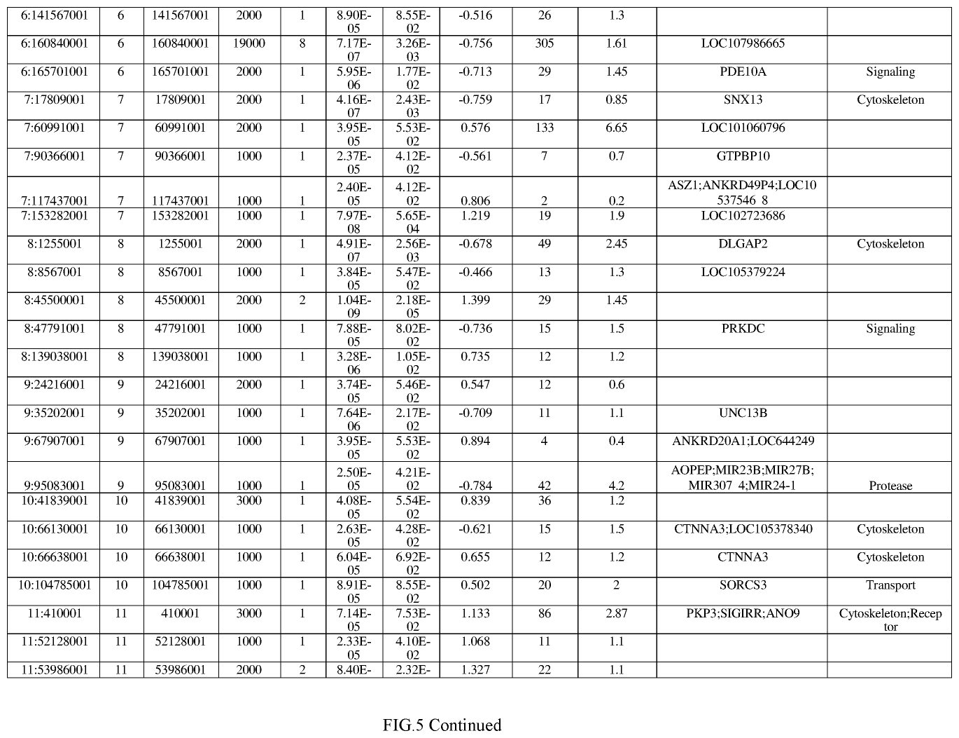

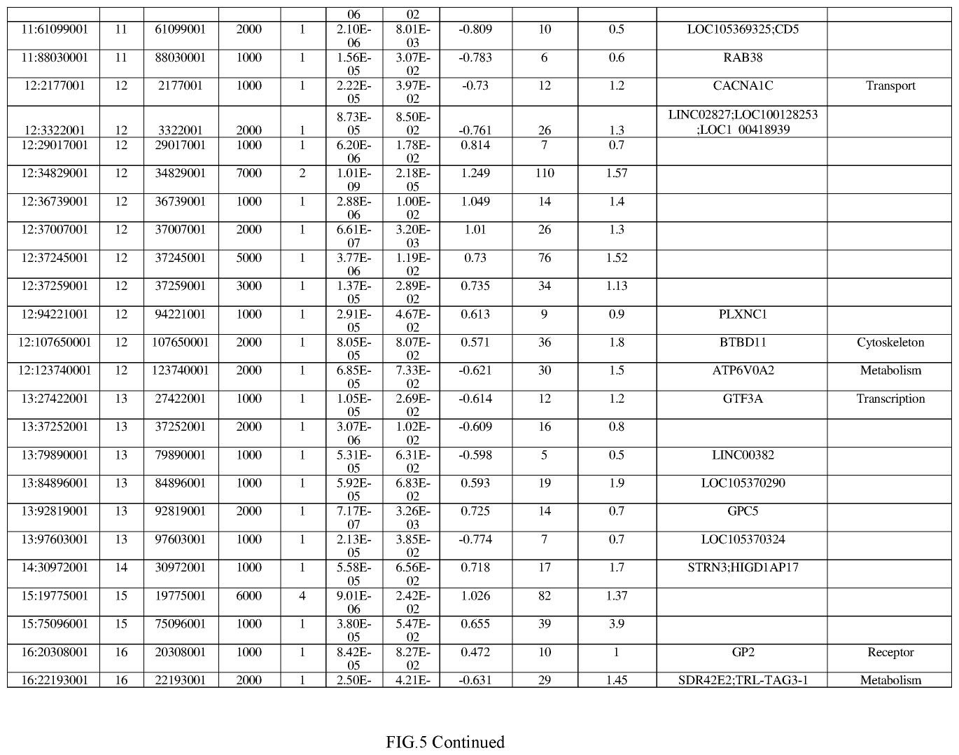

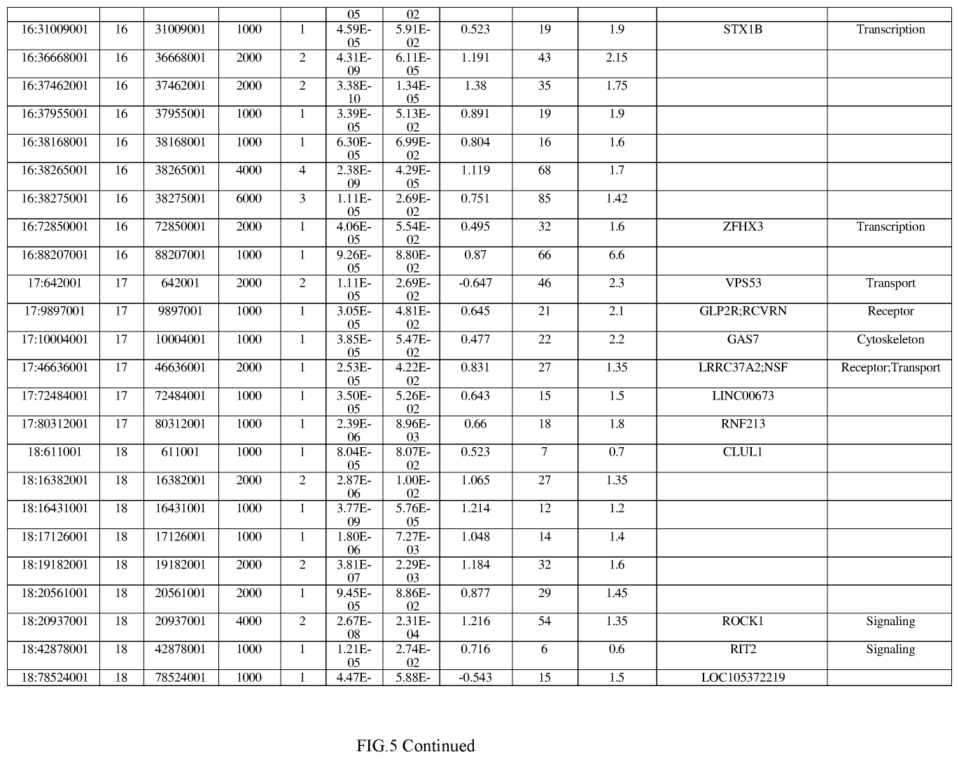

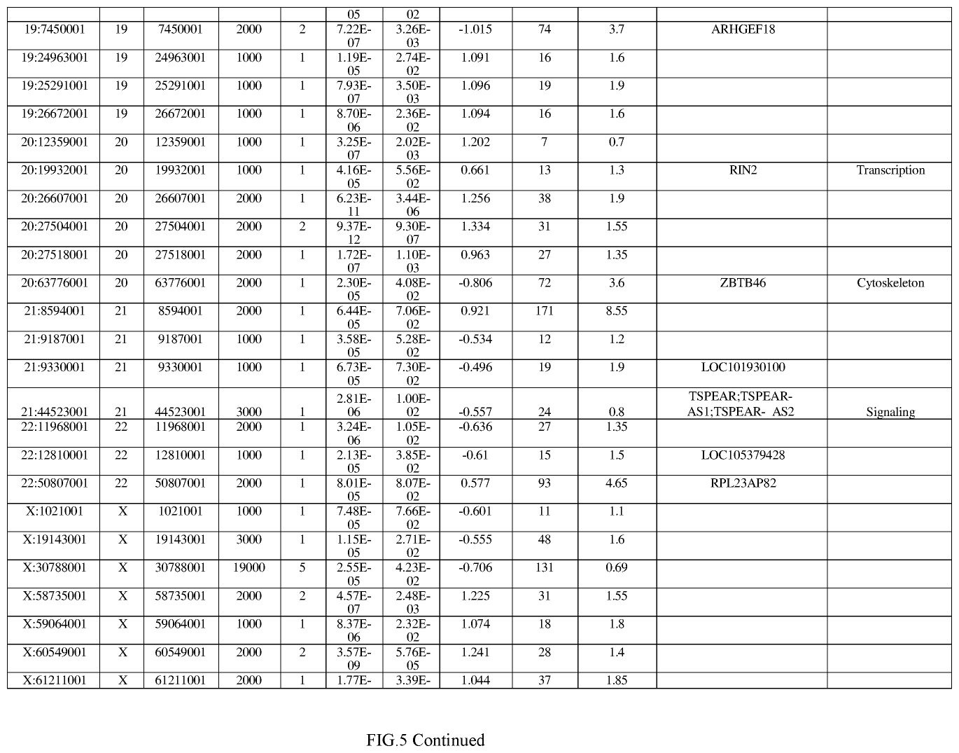

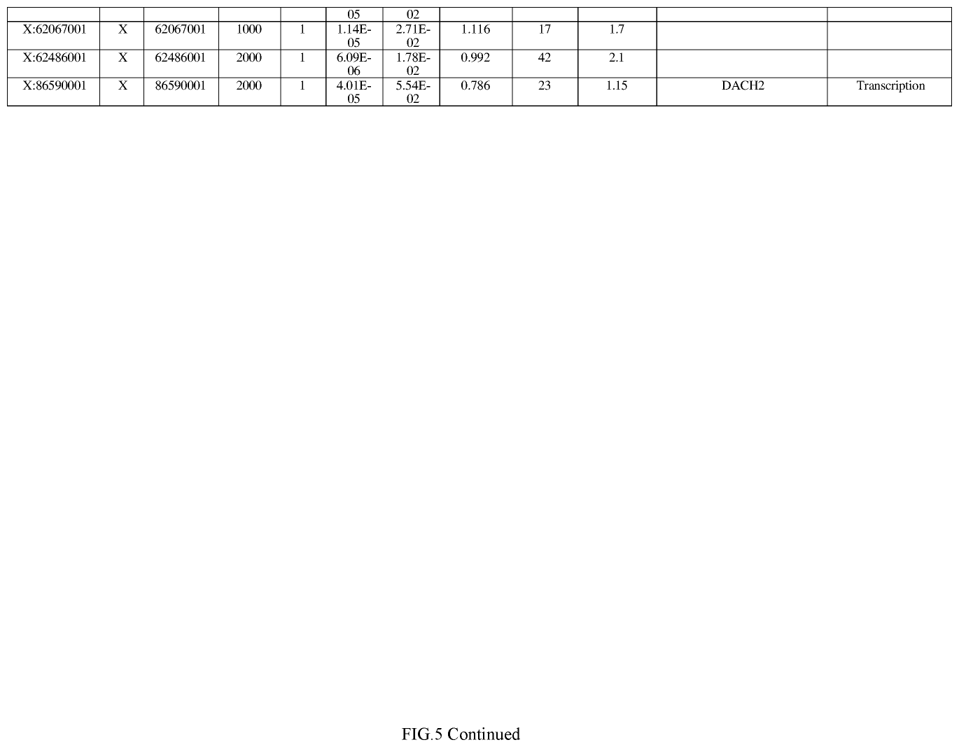

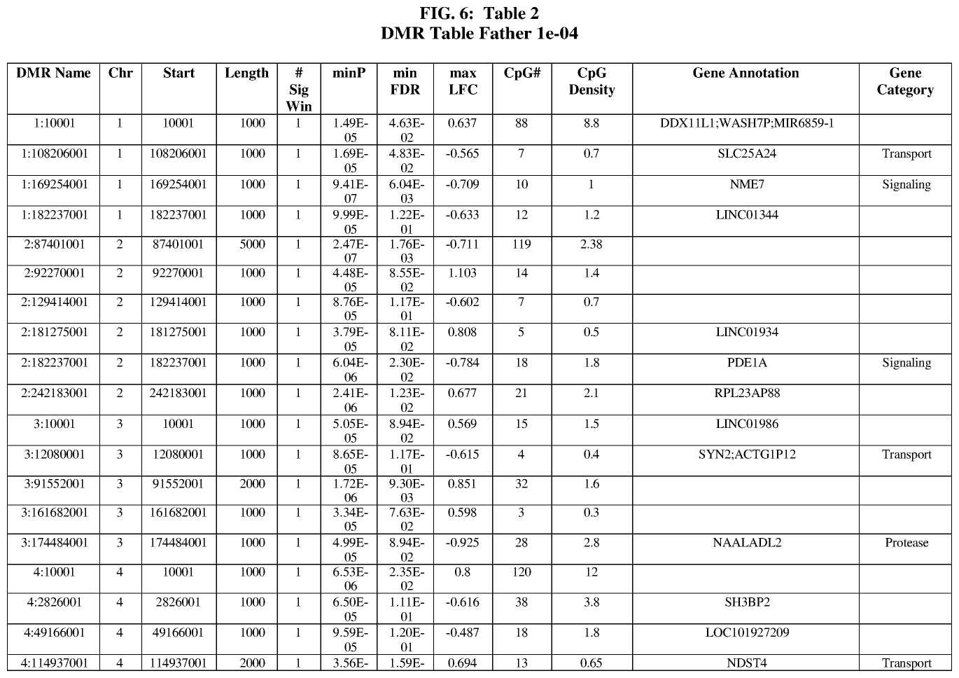

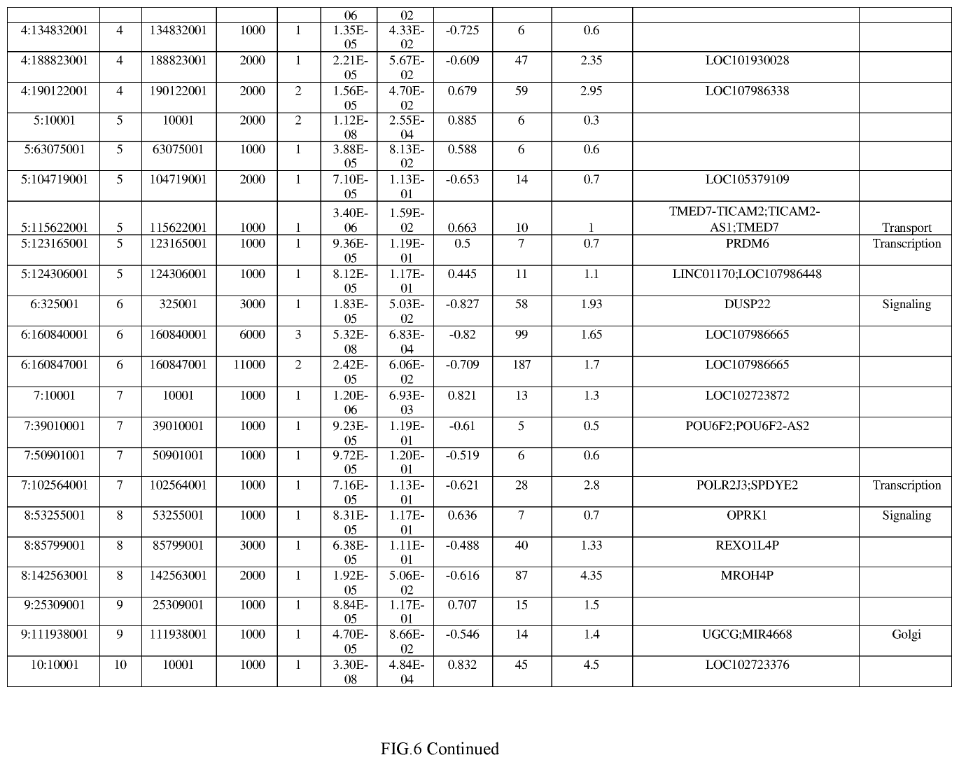

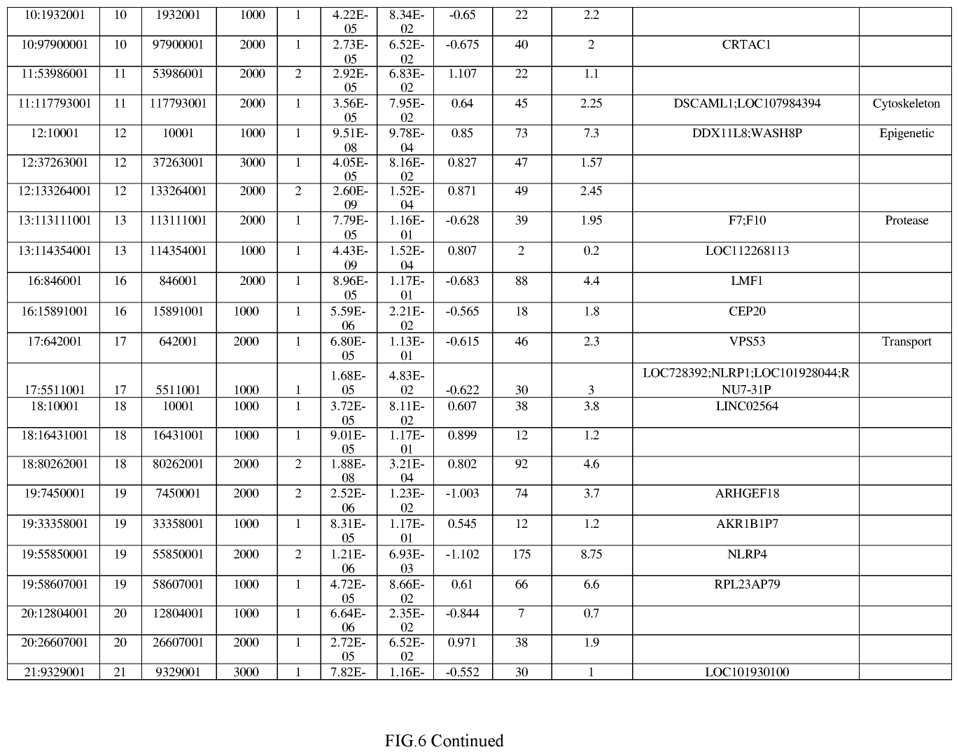

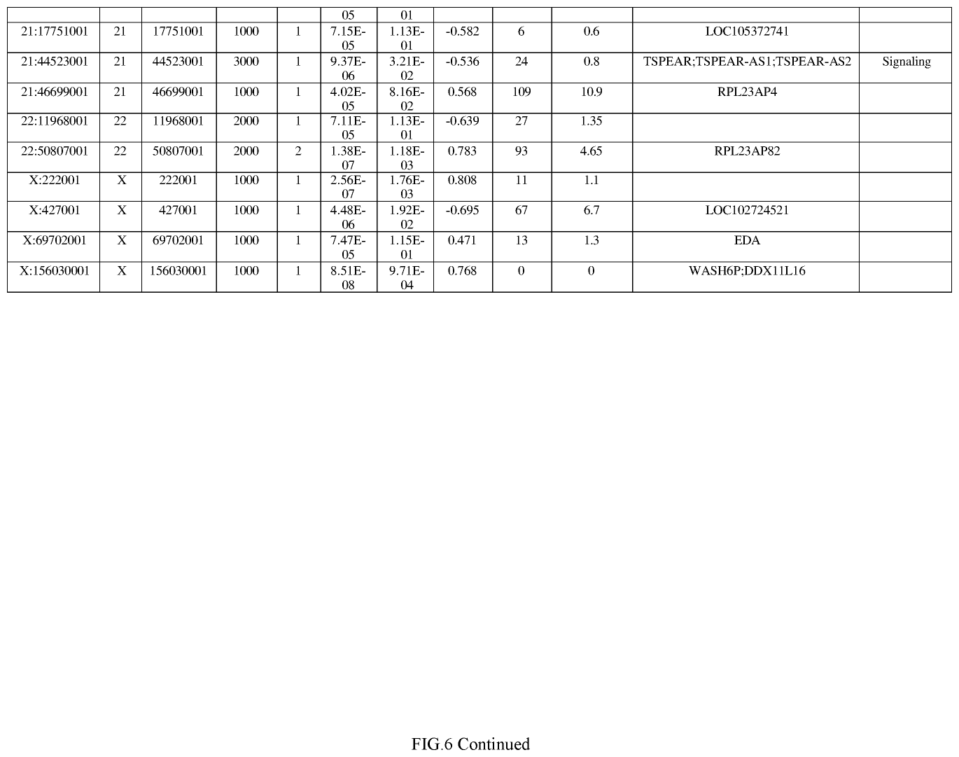

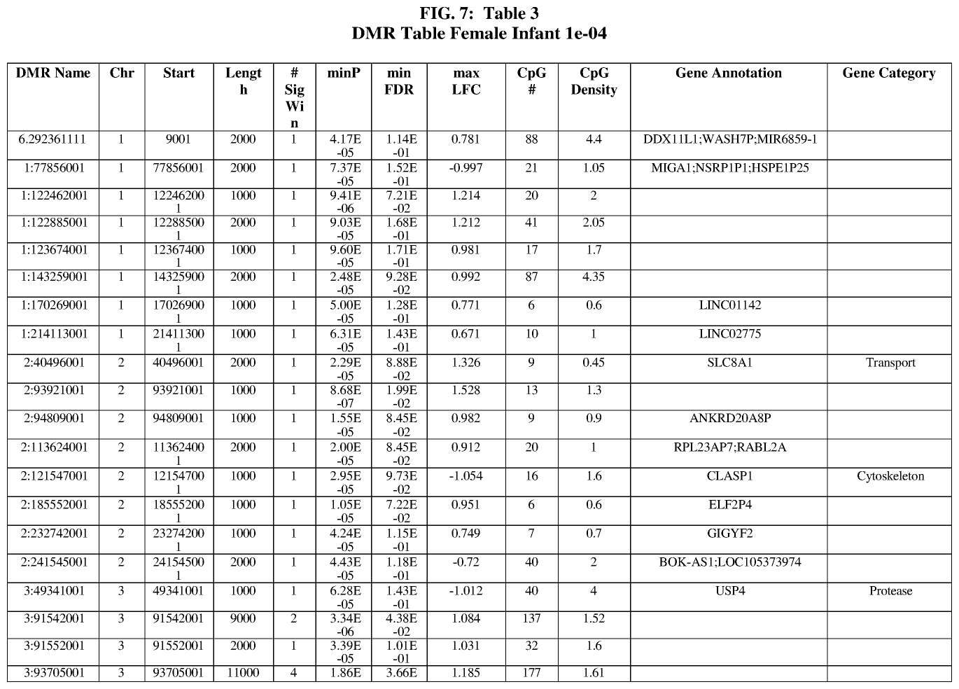

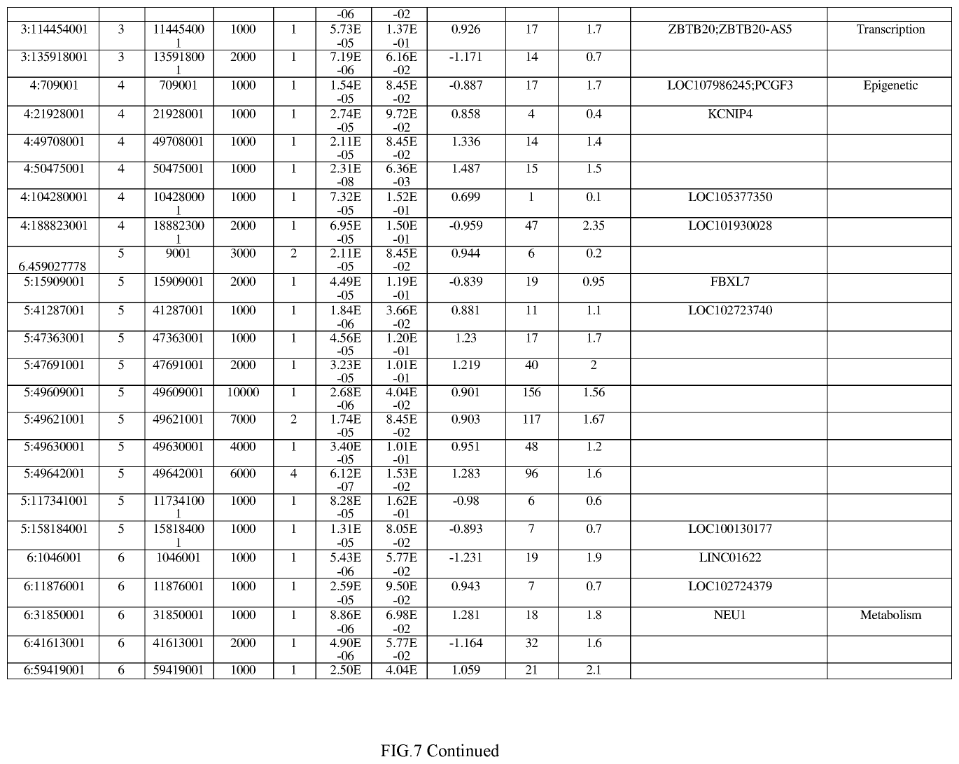

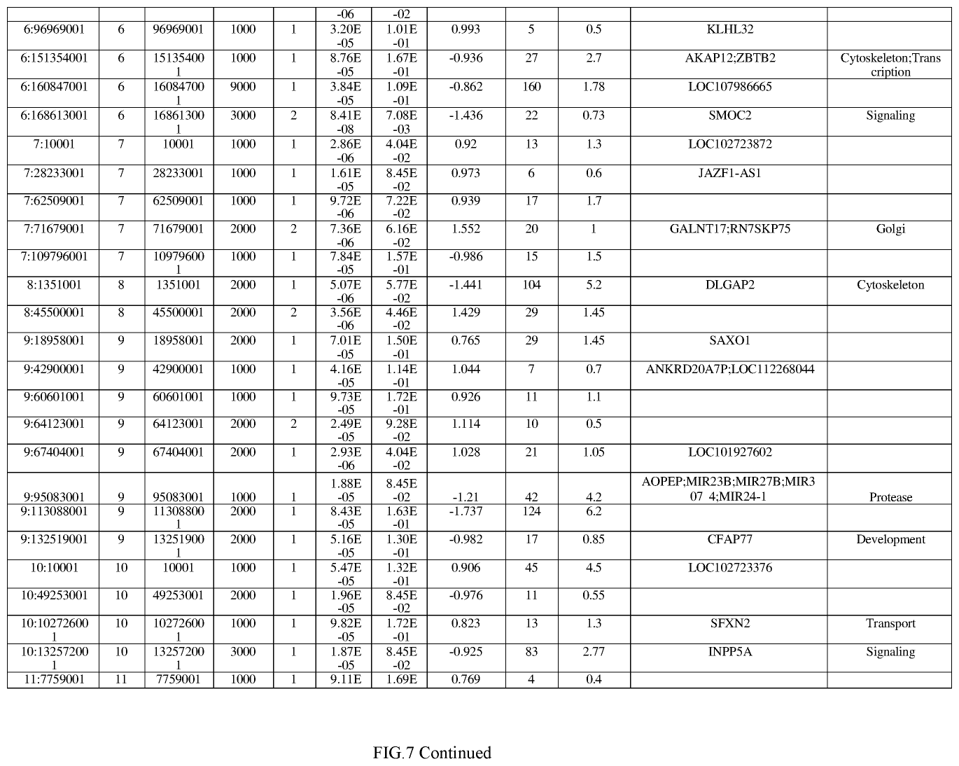

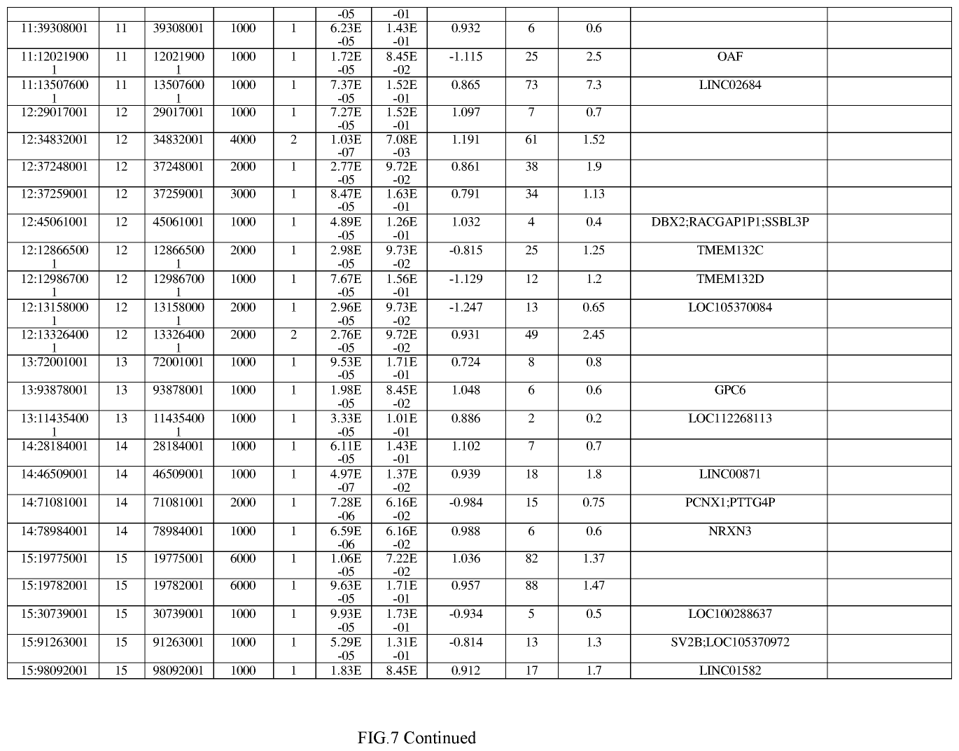

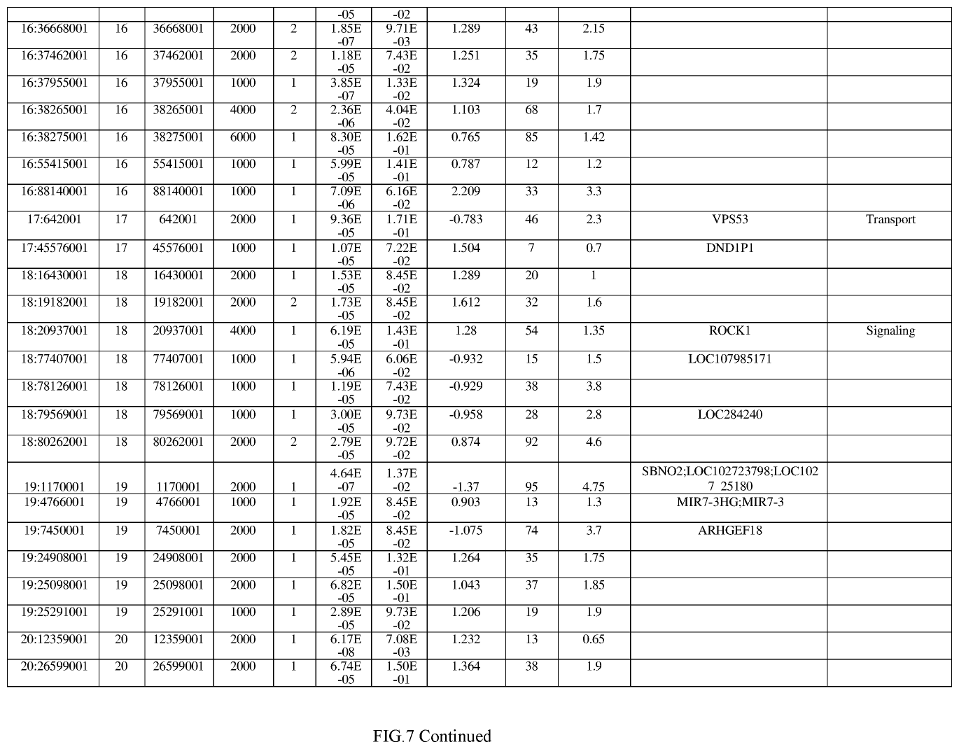

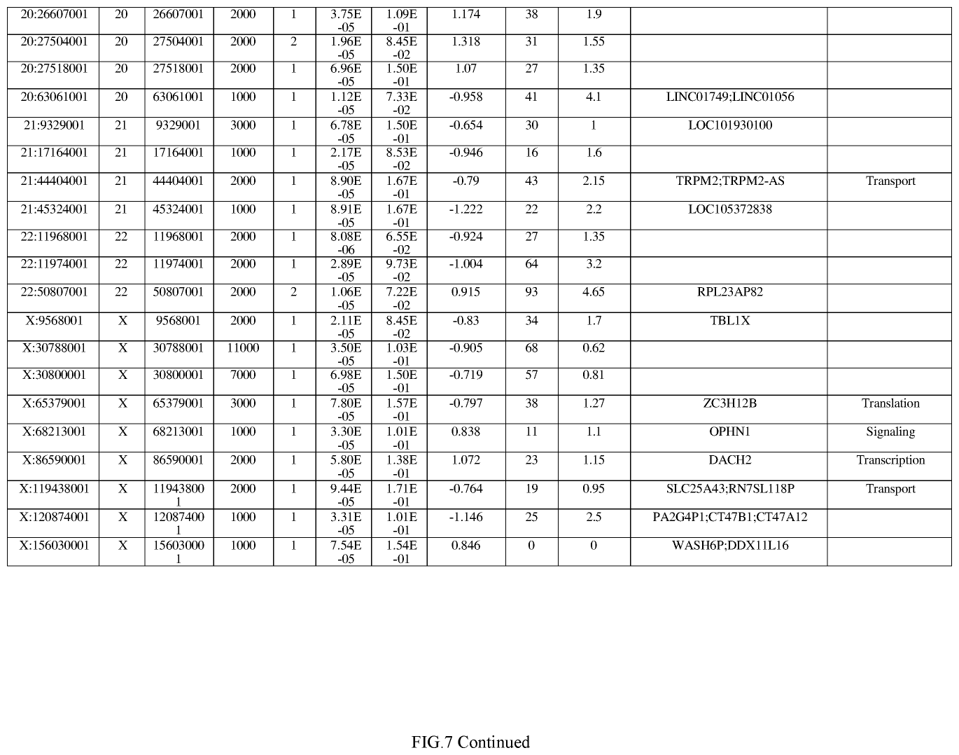

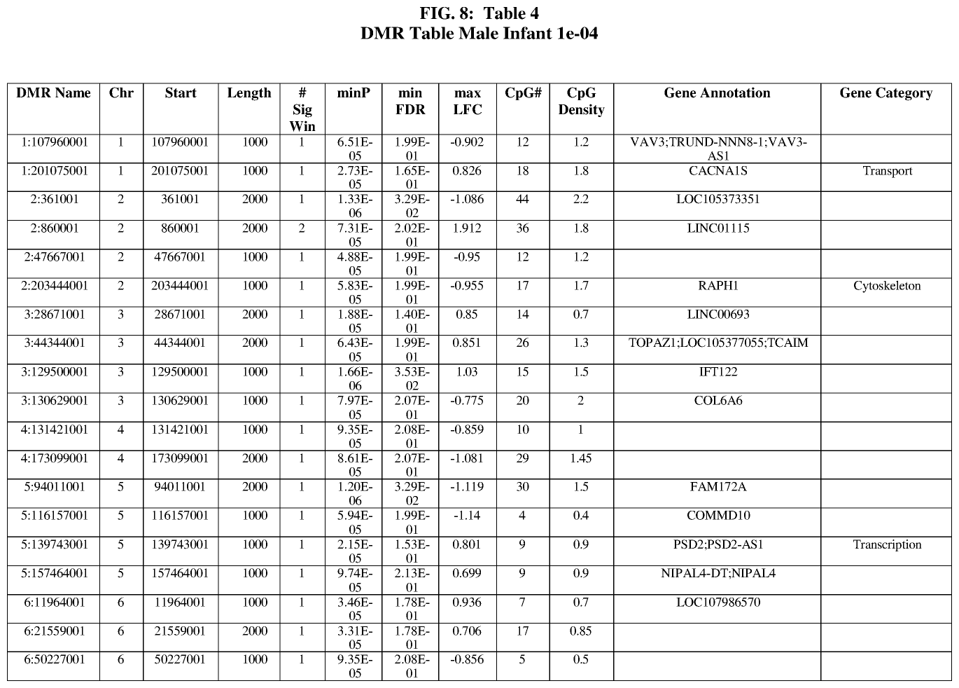

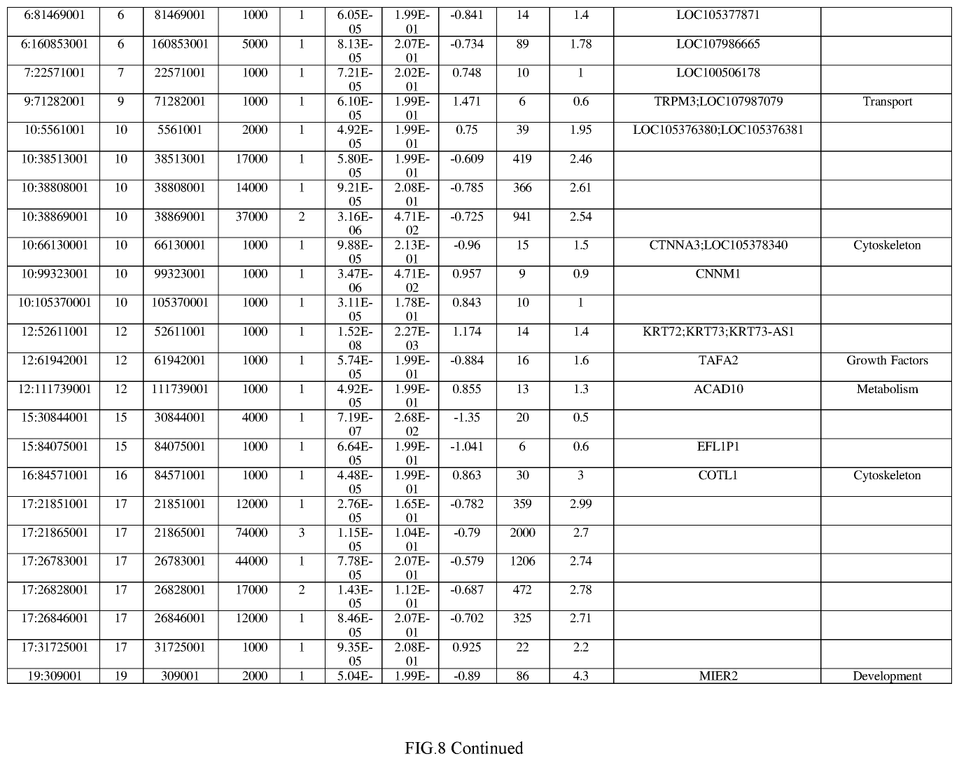

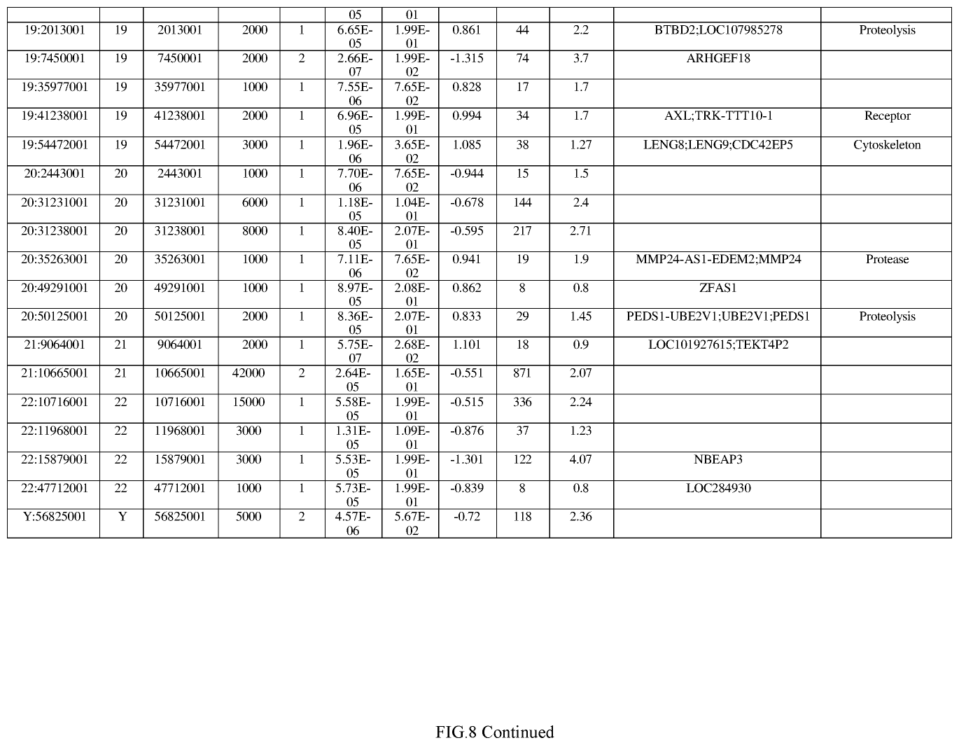

In an embodiment, the plurality of distinct DMRs are selected from the DMRs in Table 3, the DMRs in Table 2, the DMRs in Table 3, the DMRs in Table 4, and any combination thereof. In an embodiment, the plurality of distinct DMRs are selected from the DMRs in Table 1. FIG. 5 displays Table 1. In an embodiment, the plurality of distinct DMRs are selected from the DMRs in Table 2. FIG. 6 displays Table 2. In an embodiment, the plurality of distinct DMRs are selected from the DMRs in Table 3. FIG. 7 displays Table 3. In an embodiment, the plurality of distinct DMRs are selected from the DMRs in Table 4. FIG. 8 displays Table 4.

In some embodiments, a DMR is determined from a parent. In some embodiments, a DMR is determined from a mother. In some embodiments, a DMR is determined from a father. In some embodiments, a DMR is determined from a child. In some embodiments, a DMR is determined from a female child. In some embodiments, a DMR is determined from a male child.

In some embodiments, a method can comprise: obtaining a sample of somatic cells from a human subject; isolating deoxyribonucleic acid (DNA) from the sample; detecting and determining a methylation level of a differential DNA methylation region (DMR) comprised in the isolated DNA; and comparing the methylation level of the DMR to a reference methylation level of a corresponding reference DMR. In some cases, a reference DMR can be from a reference (e.g., control) sample. In some cases, a reference sample can be from a healthy population (e.g., a population that is not disposed to preterm birth) or a diseased population (e.g., a population disposed to preterm birth). In some cases, a population can be a parent population (father and/or mother), a child population (male and/or female), or any combination thereof. In some cases, the comparing comprises comparing employing a computer comprising a computer processor and computer readable memory comprising computer readable instructions. In some cases, the detecting and determining can comprise a methylated DNA immunoprecipitation (MeDIP), a sequencing, a bisulfite treatment, a bisulfite conversion, a deamination of an unmethylated cytosine base, employing an array, or any combination thereof. In some cases, the methylation level of one or more distinct DMRs (for example from the DMRs disclosed in Table 1, Table 2, Table 3, Table 4 or any combination thereof) is detected and compared. In some cases, one or more distinct DMRs are shared in Table 1, Table 2, Table 3, and Table 4. In some embodiments, one or more distinct DMRs can be selected from the DMRs in Table 1, Table 2, Table 3, Table 4 or any combination thereof. In some embodiments, a method can comprise determining a methylation level in at least one distinct DMR. In some embodiments, a distinct DMR can be selected from the DMRs in Table 1, Table 2, Table 3, Table 4 or any combination thereof. In some cases, a method can comprise determining a methylation level in at least 10 distinct DMRs. In some cases, a method can comprise determining a methylation level in more than or less than about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 distinct DMRs. In some cases, a method can comprise determining a methylation level in about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 distinct DMRs. In some cases, a method can comprise determining a methylation level in about: 1 to 250 DMRs, 1 to about 10 DMRs, 1 to about 25 DMRs, 10 to about 25 DMRs, 15 to about 30 DMRs 10 to about 50 DMRs, 20 to about 60 DMRs, 25 to about 80 DMRs, 50 to about 100 DMRs, 100 to about 150 DMRs, or about 150 to about 250 DMRs. In some embodiments, the methylation level of a sample can be hypermethylated, hypomethylated, or unchanged as compared to a reference methylation level.

In an embodiment, the plurality of distinct DMRs comprises at least 10 distinct DMRs. In an embodiment, the plurality of distinct DMRs comprises 2 to about 10 distinct DMRs. In an embodiment, the plurality of distinct DMRs comprises 5 to about 15 distinct DMRs. In an embodiment, the plurality of distinct DMRs comprises 3 to about 8 distinct DMRs. In an embodiment, the plurality of distinct DMRs comprises 4 to about 12 distinct DMRs. In an embodiment, the plurality of distinct DMRs comprises about 20 to about 30 distinct DMRs. In an embodiment, the plurality of distinct DMRs comprises about 10 to about 1000 distinct DMRs. In an embodiment, the plurality of distinct DMRs comprises about 10 to about 15 distinct DMRs. In an embodiment, the plurality of distinct DMRs comprises about 10 to about 50 distinct DMRs. In an embodiment, the plurality of distinct DMRs comprises about 10 to about 100 distinct DMRs. In an embodiment, the plurality of distinct DMRs comprises about 50 to about 100 distinct DMRs. In an embodiment, the plurality of distinct DMRs comprises about 100 to about 150 distinct DMRs. In an embodiment, the plurality of distinct DMRs comprises about 150 to about 200 distinct DMRs. In an embodiment, the plurality of distinct DMRs comprises about 200 to about 250 distinct DMRs. In an embodiment, the plurality of distinct DMRs comprises about 250 to about 300 distinct DMRs. In an embodiment, the plurality of distinct DMRs comprises about 300 to about 350 distinct DMRs. In an embodiment, the plurality of distinct DMRs comprises about 350 to about 400 distinct DMRs. In an embodiment, the plurality of distinct DMRs comprises about 400 to about 450 distinct DMRs. In an embodiment, the plurality of distinct DMRs comprises about 450 to about 500 distinct DMRs. In an embodiment, the plurality of distinct DMRs comprises about 500 to about 550 distinct DMRs. In an embodiment, the plurality of distinct DMRs comprises about 550 to about 600 distinct DMRs. In an embodiment, the plurality of distinct DMRs comprises about 600 to about 650 distinct DMRs. In an embodiment, the plurality of distinct DMRs comprises about 650 to about 700 distinct DMRs. In an embodiment, the plurality of distinct DMRs comprises about 700 to about 750 distinct DMRs. In an embodiment, the plurality of distinct DMRs comprises about 750 to about 800 distinct DMRs. In an embodiment, the plurality of distinct DMRs comprises about 800 to about 850 distinct DMRs. In an embodiment, the plurality of distinct DMRs comprises about 850 to about 900 distinct DMRs. In an embodiment, the plurality of distinct DMRs comprises about 900 to about 950 distinct DMRs. In an embodiment, the plurality of distinct DMRs comprises about 950 to about 1000 distinct DMRs.

Also disclosed herein are computer control systems that are programmed to implement methods described herein.

In some embodiments, the methods herein comprise the steps of detecting, comparing, determining, or any combination thereof. In some cases, the detecting can comprise executing a computer program on a computer. In some cases, the comparing can comprise executing a computer program on a computer. In some cases, the determining can comprise executing a computer program on a computer. In some embodiments, the detecting, the comparing, the determining, or any combination thereof employs a computer processor. In some embodiments, the detecting, the comparing, the determining, or any combination thereof employs computer readable memory. In some embodiments, the detecting, the comparing, the determining, or any combination thereof employs computer readable instructions on a computer readable memory. In some cases, the determining employs a computer processor for receiving and analyzing data comprising the presence or absence of a methylation level. In some cases, a computer program can be the same computer program. In some cases, a computer program can be a different computer program. For example, the detecting and comparing steps can use the same or different computer programs. In some cases, a computer can comprise a electronic for displaying data, a computation, an output, an input, or any combination thereof.

In some embodiments, computer systems can be used for analyzing DNA from a somatic cell DNA sample or blood cell DNA sample obtained from a subject. In some cases, the computer system can comprise: a) a device for receiving sequenced data, wherein the sequenced data comprises sequencing of at least one DMR of Table 1, Table 2, Table 3, Table 4 or any combination thereof; and b) a device for comparing the sequenced data with a control methylation level to determine the methylation level of the sequenced data. In some cases a computer system can be used to determine if a subject has or is at risk for preterm birth. In some cases, sequence data comprises data obtained from the sequencing of bisulfite converted DNA. In some cases, the methylation level comprises a hypermethylation or a hypomethylation of one or more DMRs from Table 1, Table 2, Table 3, Table 4 or any combination thereof.

In some cases, a device can be used for receiving an array and the data associated with an array. In some cases, a device can be used to compare the data from the array with a control. In some cases, the data from the array can be a methylation level. In some cases, a device can be a computer system.

In an embodiment, the plurality of distinct DMRs demonstrate an increase in DNA methylation. In an embodiment, the plurality of distinct DMRs demonstrate a decrease in DNA methylation.

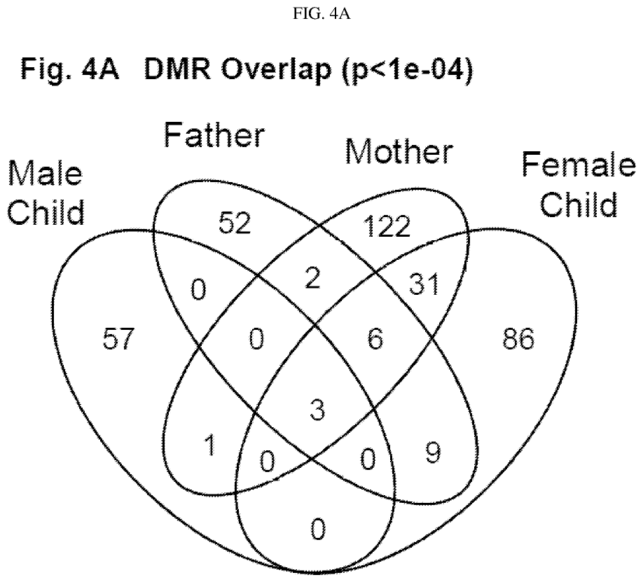

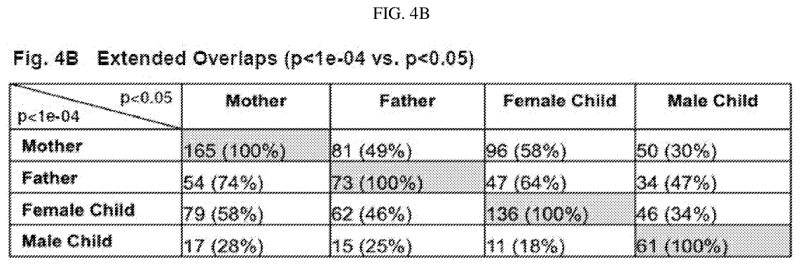

In an embodiment, the plurality of distinct DMRs are of a DMR associated gene category. Examples of DMR associated gene categories are presented in FIGS. 4A-4B.

In an embodiment, the DMR associated gene category is a signaling gene category. In an embodiment, the DMR associated gene category is a transcription gene category. In an embodiment, the DMR associated gene category is a metabolism gene category. In an embodiment, the DMR associated gene category is a receptor gene category. In an embodiment, the DMR associated gene category is a cytoskeleton gene category. In an embodiment, the DMR associated gene category is a development gene category. In an embodiment, the DMR associated gene category is a transport gene category. In an embodiment, the DMR associated gene category is an extracellular matrix gene category. In an embodiment, the DMR associated gene category is an epigenetic gene category. In an embodiment, the DMR associated gene category is a protease gene category. In an embodiment, the DMR associated gene category is a proteolysis gene category. In an embodiment, the DMR associated gene category is a translation gene category. In an embodiment, the DMR associated gene category is a binding protein gene category. In an embodiment, the DMR associated gene category is a growth factor/cytokine gene category. In an embodiment, the DMR associated gene category is a cell cycle gene category. In an embodiment, the DMR associated gene category is an immune gene category. In an embodiment, the DMR associated gene category is an apoptosis gene category. In an embodiment, the DMR associated gene category is a Golgi gene category. In an embodiment, the DMR associated gene category is an EST gene category. In an embodiment, the DMR associated gene category is an electron transport gene category. In an embodiment, the DMR associated gene category is a protein binding gene category. In an embodiment, the DMR associated gene category is a DNA repair gene category.

In an embodiment, the method comprises sequencing, wherein the sequencing comprises sequencing by synthesis, ion semiconductor sequencing, single molecule real time sequencing, nanopore sequencing, next-generation sequencing, or any combination thereof. Such sequencing procedures are well known in the art to the skilled person.

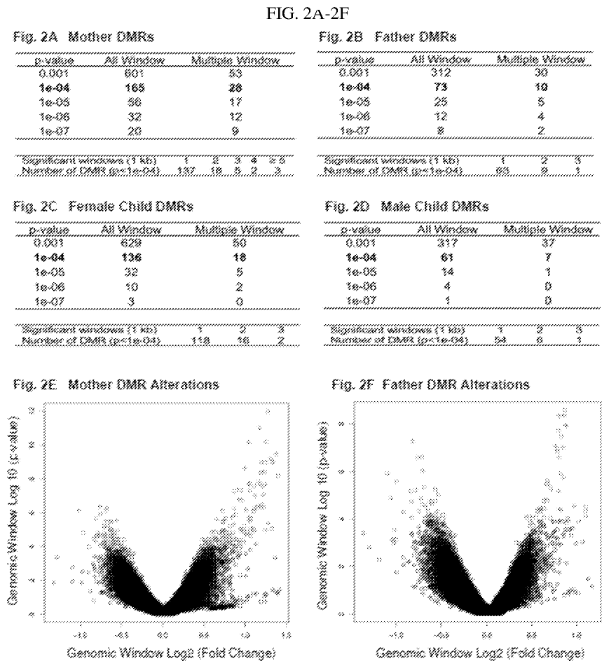

In an embodiment, the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes. Examples of chromosomal distribution of DMRs are presented in FIGS. 2A-2D.

In an embodiment, the DMRs that are determined and compared, individually, range from about 100 to about 17000 adjacent nucleotides or from about 10 to about 74,000 adjacent nucleotides. In some embodiments, the size of a DMR individually and independently can be about: 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 3000 nucleotides, 4000 nucleotides, 5000 nucleotides, 6000 nucleotides, 7000 nucleotides, 8000 nucleotides, 9000 nucleotides, 10000 nucleotides, 11000 nucleotides, 12000 nucleotides, 13000 nucleotides, 14000 nucleotides, 15000 nucleotides, 16000 nucleotides, 17000 nucleotides, 18000 nucleotides, 19000 nucleotides, 20000 nucleotides, 21000 nucleotides, 22000 nucleotides, 23000 nucleotides, 24000 nucleotides, 25000 nucleotides, 26000 nucleotides, 27000 nucleotides, 28000 nucleotides, 29000 nucleotides, 30000 nucleotides, 31000 nucleotides, 32000 nucleotides, 33000 nucleotides, 34000 nucleotides, 35000 nucleotides, 36000 nucleotides, 37000 nucleotides, 38000 nucleotides, 39000 nucleotides, 40000 nucleotides, 41000 nucleotides, 42000 nucleotides, 43000 nucleotides, 44000 nucleotides, 45000 nucleotides, 46000 nucleotides, 47000 nucleotides, 48000 nucleotides, 49000 nucleotides, 50000 nucleotides, 51000 nucleotides, 52000 nucleotides, 53000 nucleotides, 54000 nucleotides, 55000 nucleotides, 56000 nucleotides, 57000 nucleotides, 58000 nucleotides, 59000 nucleotides, 60000 nucleotides, 61000 nucleotides, 62000 nucleotides, 63000 nucleotides, 64000 nucleotides, 65000 nucleotides, 66000 nucleotides, 67000 nucleotides, 68000 nucleotides, 69000 nucleotides, 70000 nucleotides, 71000 nucleotides, 72000 nucleotides, 73000 nucleotides, 74000 nucleotides, 75000 nucleotides, or greater than 75000 nucleotides. In some embodiments, the size of a DMR individually and independently can be about: 10 nucleotides to about 75,000 nucleotides, 10 nucleotides to about 1000 nucleotides, 100 nucleotides to about 1000 nucleotides, 500 nucleotides to about 10000 nucleotides, 1000 nucleotides to about 10000 nucleotides, 5000 nucleotides to about 20000 nucleotides, 10000 nucleotides to about 40000 nucleotides, about 20000 nucleotides to about 50000 nucleotides, or about 40000 nucleotides to about 75000 nucleotides.

In an embodiment, each DMR in a plurality of the DMRs that are determined and compared independently comprise a CpG density of about 1 to about 11 CpG per 100 nucleotides. In an embodiment, each DMR in a plurality of DMRs can independently comprise a CpG density from about 0.1 to 11 CpG per 100 nucleotides. In an embodiment, each DMR in a plurality of the DMRs that are determined and compared can independently comprise a CpG density of less than about 11 CpG per 100 nucleotides. In an embodiment, each DMR in a plurality of the DMRs that are determined and compared can independently comprise a CpG density of less than about 5 CpG per 100 nucleotides. In an embodiment, each DMR ina plurality of the DMRs that are determined and compared can independently comprise a CpG density of less than about 3 CpG per 100 nucleotides. In an embodiment, each DMR in a plurality of the DMRs that are determined and compared can independently comprise a CpG density of from 1 CpG and 3 CpG per 100 nucleotides. In an embodiment, each DMR in a plurality of the DMRs that are determined and compared can independently comprise a CpG density of from about 0.1 CpG to about 3 CpG per 100 nucleotides. In some embodiments, each DMR in a plurality of DMRs can independently comprise a CpG density from about: 1 CpG to 50 CpG per 100 nucleotides, 0.1 CpG to 50 CpG per 100 nucleotides, 1 CpG to 40 CpG per 100 nucleotides, 1 CpG to 30 CpG per 100 nucleotides, 1 CpG to 20 CpG per 100 nucleotides, 2 CpG to 9 CpG per 100 nucleotides, 3 CpG to 8 CpG per 100 nucleotides, 4 CpG to 7 CpG per 100 nucleotides, 5 CpG to 15 CpG per 100 nucleotides, 10 CpG to 20 CpG per 100 nucleotides, or 15 CpG to 25 CpG per 100 nucleotides. In some embodiments, a CpG density in a DMR can be about: 0.1 CpG per 100 nucleotides, 0.2 CpG per 100 nucleotides, 0.3 CpG per 100 nucleotides, 0.4 CpG per 100 nucleotides, 0.5 CpG per 100 nucleotides, 0.6 CpG per 100 nucleotides, 0.7 CpG per 100 nucleotides, 0.8 CpG per 100 nucleotides, 0.9 CpG per 100 nucleotides, 1 CpG per 100 nucleotides, 2 CpG per 100 nucleotides, 3 CpG per 100 nucleotides, 4 CpG per 100 nucleotides, 5 CpG per 100 nucleotides, 6 CpG per 100 nucleotides, 7 CpG per 100 nucleotides, 8 CpG per 100 nucleotides, 9 CpG per 100 nucleotides, 10 CpG per 100 nucleotides, or 11 CpG per 100 nucleotides.

In some embodiments, a number of CpG per individual DMR is determined. In some cases, more than, less than, or equal to about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, or 3000 CpG(s) per individual DMR are determined. In some cases, about: 1 to about 3000 CpGs per individual DMR are determined, 1 to about 10 CpGs per individual DMR are determined, 1 to about 100 CpGs per individual DMR are determined, 10 to about 50 CpGs per individual DMR are determined, 20 to about 80 CpGs per individual DMR are determined, 60 to about 200 CpGs per individual DMR are determined, 100 to about 500 CpGs per individual DMR are determined, 250 to about 1000 CpGs per individual DMR are determined, or 1000 to about 3000 CpGs per individual DMR are determined. In some cases, methylation can comprise methylation at a CpG site. For example, cytosines in CpG dinucleotides can be methylated to form 5-methylcytosine by methyltransferases.

In an embodiment, at least about or about: 10, 20, 30, 40, 50, 60, 70, 80, or 90 percent of the DMRs that are determined and compared are hypermethylated when compared, individually, to individual reference methylation levels of corresponding individual reference DMRs. In an embodiment, at least about or about: 10, 20, 30, 40, 50, 60, 70, 80, or 90 percent of the DMRs that are determined and compared are hypomethylated when compared, individually, to individual reference methylation levels of corresponding individual reference DMRs. In an embodiment, all of the DMRs that are determined and compared are hypomethylated when compared, individually, to individual reference methylation levels of corresponding individual reference DMRs. In an embodiment, all of the DMRs that are determined and compared are hypermethylated when compared, individually, to individual reference methylation levels of corresponding individual reference DMRs.

In an embodiment, a gene or a portion thereof can be hypermethylation or hypomethylated. In some cases, a gene or a portion thereof can be a gene annotated in Table 1, Table 2, Table 3, Table 4, or any combination thereof. In some cases, a hypomethylated gene or a portion thereof or a hypermethylated gene or a portion thereof can individually be selected from in Table 1, Table 2, Table 3, or Table 4.

In an embodiment, the human subject is a male subject. In an embodiment, the human subject is a female subject. In an embodiment, the human subject is a female infant. In an embodiment, the human subject is a male infant.

In an embodiment, the method further comprises determining a risk of the human subject for preterm birth, wherein the determining is performed with a computer comprising a computer processor and computer readable memory comprising computer readable instructions.

In an embodiment, the method further comprises performing an additional analysis using a computer. In an embodiment, the additional analysis comprises a principle component analysis (PCA), a dendrogram analysis, a machine learning analysis, or any combination thereof.

In an embodiment, the additional analysis generates data points, and wherein the data points in the additional analysis are grouped into two spatially distinct categories comprising a first category and a second category. In an embodiment, the first category indicates the human subject is at increased risk of having preterm birth and wherein the second category indicates the human subject is not at increased risk of having preterm birth.

In an embodiment, the method further comprises treating the human subject. In an embodiment, the treating comprises administering clinical management for prevention of preterm birth. In an embodiment, the clinical management comprises enhanced monitoring of pregnancy. In an embodiment, the clinical management comprises application of protective uterine monitoring. In an embodiment, the clinical management comprises hospital admission of the human subject. In an embodiment, the clinical management comprises operative delivery performed on the human subject.

In an embodiment, the treating comprises administering a medication for preventative treatment of preterm birth. In an embodiment, the medication is a prenatal steroid. In an embodiment, the medication is a tocolytic. In an embodiment, the tocolytic is an oxytocin receptor (OTR) antagonist. In an embodiment, the tocolytic is selected from the group consisting of a β2 agonist, a calcium channel blocker, a nonsteroidal anti-inflammatory drug (NSAID), magnesium sulfate, and any combination thereof. In an embodiment, the tocolytic is a β2 agonist. In an embodiment, the tocolytic is a calcium channel blocker. In an embodiment, the tocolytic is an NSAID. In an embodiment, the tocolytic is magnesium sulfate. In an embodiment, a tocolytic can be a beta-adrenergic receptor agonist. In an embodiment, a tocolytic can be a oxytocin inhibitor. In some cases, a beta-adrenergic receptor can comprise terbutaline, hexoprenaline, ritodrine, a salt of any of these, or any combination thereof. In some cases, a calcium channel blocker can comprise nifedipine or a salt thereof. In some cases, a nonsteroidal anti-inflammatory drug can comprise indomethacin, or a salt thereof. In some cases, a oxytocin inhibitor can comprise atosiban, retosiban, a salt of any of these, or any combination thereof. In some cases, a preventative treatment of preterm birth can comprise nitroglycerine.

In an embodiment, the tocolytic is terbutaline. In an embodiment, the tocolytic is ritodrine. In an embodiment, the tocolytic is fenoterol. In an embodiment, the tocolytic is albuterol. In an embodiment, the tocolytic is hexoprenaline. In an embodiment, the tocolytic is nifedipine. In an embodiment, the tocolytic is atosiban. In an embodiment, the tocolytic is indomethacin. In an embodiment, the tocolytic is sulindac.

In some embodiments, methods described herein can comprise administering a therapy (e.g., treatment) to a subject in need thereof, for example a subject at risk for preterm birth.

In some embodiments, the terms “administer,” “administering”, “administration,” and the like, as used herein, can refer to methods that can be used to deliver therapies described herein. In some cases, delivery can include injection, inhalation, catheterization, gastrostomy tube administration, intravenous administration, intraosseous administration, ocular administration, otic administration, topical administration, transdermal administration, oral administration, rectal administration, nasal administration, intravaginal administration, intracavernous administration, intracerebral administration, transurethral administration, buccal administration, sublingual administration, intrapenile drug delivery, subcutaneous administration, or a combination thereof. Delivery can include a parenchymal injection, an intra-thecal injection, an intra-ventricular injection, or an intra-cisternal injection. A therapy provided herein can be administered by any method. In some cases, a medical professional can administer the therapy described herein.

Administration of a therapy disclosed herein can be performed for a treatment duration of at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, or more consecutive or nonconsecutive days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days.

Administration or application of a therapy disclosed herein can be performed for a treatment duration of at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, or for life. In some embodiments, administering can be performed for about: 1 day to about 8 days, 1 week to about 5 weeks, 1 month to about 12 months, or 1 year to about 3 years.

Administration can be performed repeatedly over a lifetime of a subject, such as once a month or once a year for the lifetime of a subject.

Administration or application of a therapy disclosed herein can be performed at least 1, 2, 3, 4, 5, 6, 7, 8, 9, 10, 11, 12, 13, 14, 15, 16, 17, 18, 19, 20, 21, 22, 23, or 24 times a in a 24 hour period. In some cases, administration or application of a therapy disclosed herein can be performed continuously throughout a 24 hour period. In some cases, administration or application of a therapy disclosed herein can be performed at least 1, 2, 3, 4, 5, 6, 7, 8, 9, 10, or more times a week. In some cases, administration or application of a therapy disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more times a month. In some cases, a therapy can be administered as a single dose or as divided doses. In some cases, a therapy described herein can be administered at a first time point and a second time point.

In some cases, a therapy herein can be administered at a dose of about 0.0001 grams to about 1000 grams. In some cases a therapy herein can be administered at a dose of about 1 mg to about 1 gram. In some cases, a therapy herein can be administered at a dose of about: 10 μg, 100 μg, 500 μg 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or 100 mg. In some cases, a therapy herein can be a pharmaceutical composition. In some cases, a therapy or a pharmaceutical composition can be in unit dose form.

In some cases, a therapy can be administered with an excipient, a carrier, a diluent or any combination thereof. In some cases, a carrier, a diluent, or both, can comprise water, saline, or any pharmaceutically acceptable carrier and/or diluent. In some cases, a diluent can comprise a pH buffer. In some cases, an excipient, carrier, or diluent can comprise water.

In an embodiment, the method further comprises transmitting data, a result, or both, via an electronic communication medium.

In an illustrative aspect, a second method is provided. The method comprises obtaining a sample of a somatic cell from a human subject; isolating deoxyribonucleic acid (DNA) from the sample, thereby obtaining isolated DNA; detecting and determining a methylation level of a differential DNA methylation region (DMR) comprised in the isolated DNA; and comparing the methylation level of the DMR to a reference methylation level of a corresponding reference DMR; wherein the comparing comprises employing a computer comprising a computer processor and computer readable memory comprising computer readable instructions contained thereon, wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and wherein a plurality of determined DMRs are sufficient to determine, from a process comprising the comparing and employing a computer, whether the human subject has or is at increased risk of preterm birth.

The previously described embodiments of the first method are applicable to the second method as described herein.

In an illustrative aspect, a first kit is provided. The kit comprises at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence selected from the DMRs in Table 1, the DMRs in Table 2, the DMRs in Table 3, the DMRs in Table 4, and any combination thereof; and a container.

In an embodiment, the distinct primers or pairs of primers each further comprise a unique barcode. In an embodiment, the distinct primers or pairs of primers are not bound to an array or a microarray. In an embodiment, the distinct primers or pairs of primers are bound to an array or a microarray. In an embodiment, the distinct primers or pairs of primers comprise DNA.

In an embodiment, the distinct DMR sequences are selected from the DMRs in Table 1. In an embodiment, the distinct DMR sequences are selected from the DMRs in Table 2. In an embodiment, the distinct DMR sequences are selected from the DMRs in Table 3. In an embodiment, the distinct DMR sequences are selected from the DMRs in Table 4.

In an embodiment, the distinct DMR sequences comprise from about 50 to about 100 DMRs. In an embodiment, the distinct DMR sequences comprise from about 5 to about 25 DMRs. In an embodiment, the distinct DMR sequences comprise from about 1 to about 10 DMRs. In an embodiment, the distinct DMR sequences comprise from about 10 to about 50 DMRs. In an embodiment, the distinct DMR sequences comprise from about 30 to about 80 DMRs. In an embodiment, the distinct DMR sequences comprise from about 100 to about 150 DMRs. In an embodiment, the distinct DMR sequences comprise from about 150 to about 200 DMRs. In an embodiment, the distinct DMR sequences comprise from about 200 to about 250 DMRs. In an embodiment, the distinct DMR sequences comprise from about 250 to about 300 DMRs. In an embodiment, the distinct DMR sequences comprise from about 300 to about 350 DMRs. In an embodiment, the distinct DMR sequences comprise from about 350 to about 400 DMRs. In an embodiment, the distinct DMR sequences comprise from about 400 to about 450 DMRs. In an embodiment, the distinct DMR sequences comprise from about 450 to about 500 DMRs. In an embodiment, the distinct DMR sequences comprise from about 500 to about 550 DMRs. In an embodiment, the distinct DMR sequences comprise from about 550 to about 600 DMRs. In an embodiment, the distinct DMR sequences comprise from about 600 to about 650 DMRs. In an embodiment, the distinct DMR sequences comprise from about 650 to about 700 DMRs. In an embodiment, the distinct DMR sequences comprise from about 700 to about 750 DMRs. In an embodiment, the distinct DMR sequences comprise from about 750 to about 800 DMRs. In an embodiment, the distinct DMR sequences comprise from about 800 to about 850 DMRs. In an embodiment, the distinct DMR sequences comprise from about 850 to about 900 DMRs. In an embodiment, the distinct DMR sequences comprise from about 900 to about 950 DMRs. In an embodiment, the distinct DMR sequences comprise from about 950 to about 1000 DMRs.

In an embodiment, the distinct DMR sequences are of a DMR associated gene category. In an embodiment, the DMR associated gene category is a signaling gene category.

In an embodiment, the DMR associated gene category is a transcription gene category. In an embodiment, the DMR associated gene category is a metabolism gene category. In an embodiment, the DMR associated gene category is a receptor gene category. In an embodiment, the DMR associated gene category is a cytoskeleton gene category. In an embodiment, the DMR associated gene category is a development gene category. In an embodiment, the DMR associated gene category is a transport gene category. In an embodiment, the DMR associated gene category is an extracellular matrix gene category. In an embodiment, the DMR associated gene category is an epigenetic gene category. In an embodiment, the DMR associated gene category is a protease gene category. In an embodiment, the DMR associated gene category is a proteolysis gene category. In an embodiment, the DMR associated gene category is a translation gene category. In an embodiment, the DMR associated gene category is a binding protein gene category. In an embodiment, the DMR associated gene category is a growth factor/cytokine gene category. In an embodiment, the DMR associated gene category is a cell cycle gene category. In an embodiment, the DMR associated gene category is an immune gene category. In an embodiment, the DMR associated gene category is an apoptosis gene category. In an embodiment, the DMR associated gene category is a Golgi gene category. In an embodiment, the DMR associated gene category is an EST gene category. In an embodiment, the DMR associated gene category is an electron transport gene category. In an embodiment, the DMR associated gene category is a protein binding gene category. In an embodiment, the DMR associated gene category is a DNA repair gene category.

In an illustrative aspect, a second kit is provided. The kit comprises at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct probes, each distinct probe complementary to a distinct DMR sequence selected from the DMRs in Table 1, the DMRs in Table 2, the DMRs in Table 3, the DMRs in Table 4, and any combination thereof; and a container.

The previously described embodiments of the first kit are applicable to the second kit as described herein. In an embodiment, the distinct probes further comprise at least one of fluorophore, a chromophore, a barcode, or any combination thereof. In an embodiment, each probe comprises a unique fluorophore, a chromophore, barcode, or any combination thereof.

In an illustrative aspect, a third method is provided. The method comprises: obtaining a sample of a somatic cell from a human subject; isolating deoxyribonucleic acid (DNA) from the sample, thereby obtaining isolated DNA; fragmenting the DNA; isolating fragmented methylated DNA; detecting and determining a methylation level of a differential DNA methylation region (DMR) comprised in the isolated fragmented methylated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises i) amplifying the isolated fragmented methylated DNA, ii) sequencing the isolated fragmented methylated DNA, an amplicon thereof, or both, or iii) employing an array, or iv) any combination thereof, wherein a plurality of distinct DMRs are detected and compared, wherein the plurality comprises at least 10 distinct DMRs.

The previously described embodiments of the first method and the second method are applicable to the third method as described herein. In an embodiment, the fragmenting is performed via sonication. In an embodiment, the isolating the fragmented methylated DNA comprises methylated DNA immunoprecipitation (MeDIP).

In an illustrative aspect, a fourth method is provided. The method comprises: obtaining a sample of a somatic cell from a human subject; isolating deoxyribonucleic acid (DNA) from the sample, thereby obtaining isolated DNA; fragmenting the DNA; isolating fragmented methylated DNA; detecting and determining a methylation level of a differential DNA methylation region (DMR) comprised in the isolated fragmented methylated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises i) amplifying the isolated fragmented methylated DNA, ii) sequencing the isolated fragmented methylated DNA, an amplicon thereof, or both, or iii) employing an array, or iv) any combination thereof; and wherein a plurality of determined DMRs are sufficient to determine, from a process comprising the comparing and employing a computer, whether the human subject has or is at increased risk of preterm birth.

The previously described embodiments of the first method, the second method, and the third method are applicable to the fourth method as described herein. In an embodiment, the fragmenting is performed via sonication. In an embodiment, the isolating the fragmented methylated DNA comprises methylated DNA immunoprecipitation (MeDIP).

The following numbered embodiments are contemplated and are non-limiting:
1. A method comprising:
   obtaining a sample of a somatic cell from a human subject;
   isolating deoxyribonucleic acid (DNA) from the sample, thereby obtaining isolated DNA;
   detecting and determining a methylation level of a differential DNA methylation region (DMR) comprised in the isolated DNA; and
   comparing the methylation level of the DMR to a reference methylation level of a corresponding reference DMR;
   wherein the comparing comprises comparing employing a computer comprising a computer processor and computer readable memory comprising computer readable instructions contained thereon,
   wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
   wherein a plurality of distinct DMRs are detected and compared, wherein the plurality comprises at least 10 distinct DMRs.
2. The method of clause 1, any other suitable clause, or any combination of suitable clauses, wherein the somatic cell comprises a buccal cell.
3. The method of clause 1, any other suitable clause, or any combination of suitable clauses, wherein the somatic cell consists essentially of a buccal cell.
4. The method of clause 1, any other suitable clause, or any combination of suitable clauses, wherein the somatic cell consists of a buccal cell.
5. The method of clause 1, any other suitable clause, or any combination of suitable clauses, wherein the somatic cell comprises a blood monocyte cell.
6. The method of clause 5, any other suitable clause, or any combination of suitable clauses, wherein the blood monocyte cell is a purified blood monocyte cell.
7. The method of clause 1, any other suitable clause, or any combination of suitable clauses, wherein the somatic cell consists essentially of a blood monocyte cell.
8. The method of clause 7, any other suitable clause, or any combination of suitable clauses, wherein the blood monocyte cell is a purified blood monocyte cell.
9. The method of clause 1, any other suitable clause, or any combination of suitable clauses, wherein the somatic cell consists of a blood monocyte cell.
10. The method of clause 9, any other suitable clause, or any combination of suitable clauses, wherein the blood monocyte cell is a purified blood monocyte cell.
11. The method of clause 1, any other suitable clause, or any combination of suitable clauses, wherein the somatic cell comprises a muscle cell, a blood cell, a skin cell, a nerve cell, or any combination thereof.
12. The method of clause 1, any other suitable clause, or any combination of suitable clauses, wherein the somatic cell comprises an immune cell.
13. The method of clause 1, any other suitable clause, or any combination of suitable clauses, wherein the somatic cell comprises a muscle cell.
14. The method of clause 1, any other suitable clause, or any combination of suitable clauses, wherein the somatic cell comprises a blood cell.
15. The method of clause 1, any other suitable clause, or any combination of suitable clauses, wherein the somatic cell comprises a skin cell.
16. The method of clause 1, any other suitable clause, or any combination of suitable clauses, wherein the somatic cell comprises a nerve cell.
17. The method of clause 1, any other suitable clause, or any combination of suitable clauses, wherein the plurality of distinct DMRs are selected from the DMRs in Table 1, the DMRs in Table 2, the DMRs in Table 3, the DMRs in Table 4, and any combination thereof.
18. The method of clause 1, any other suitable clause, or any combination of suitable clauses, wherein the plurality of distinct DMRs are selected from the DMRs in Table 1.
19. The method of clause 1, any other suitable clause, or any combination of suitable clauses, wherein the plurality of distinct DMRs are selected from the DMRs in Table 2.
20. The method of clause 1, any other suitable clause, or any combination of suitable clauses, wherein the plurality of distinct DMRs are selected from the DMRs in Table 3.
21. The method of clause 1, any other suitable clause, or any combination of suitable clauses, wherein the plurality of distinct DMRs are selected from the DMRs in Table 4.
22. The method of clause 1, any other suitable clause, or any combination of suitable clauses, wherein the plurality of distinct DMRs comprises at least 10 distinct DMRs.
23. The method of clause 1, any other suitable clause, or any combination of suitable clauses, wherein the plurality of distinct DMRs comprises from 2 to about 10 distinct DMRs.
24. The method of clause 1, any other suitable clause, or any combination of suitable clauses, wherein the plurality of distinct DMRs comprises from 5 to about 15 distinct DMRs.
25. The method of clause 1, any other suitable clause, or any combination of suitable clauses, wherein the plurality of distinct DMRs comprises from 3 to about 8 distinct DMRs.
26. The method of clause 1, any other suitable clause, or any combination of suitable clauses, wherein the plurality of distinct DMRs comprises from 4 to about 12 distinct DMRs.
27. The method of clause 1, any other suitable clause, or any combination of suitable clauses, wherein the plurality of distinct DMRs comprises from about 20 to about 30 distinct DMRs.
28. The method of clause 1, any other suitable clause, or any combination of suitable clauses, wherein the plurality of distinct DMRs comprises from about 10 to about 1000 distinct DMRs.
29. The method of clause 1, any other suitable clause, or any combination of suitable clauses, wherein the plurality of distinct DMRs comprises from about 10 to about 15 distinct DMRs.
30. The method of clause 1, any other suitable clause, or any combination of suitable clauses, wherein the plurality of distinct DMRs comprises from about 10 to about 50 distinct DMRs.
31. The method of clause 1, any other suitable clause, or any combination of suitable clauses, wherein the plurality of distinct DMRs comprises from about 10 to about 100 distinct DMRs.
32. The method of clause 1, any other suitable clause, or any combination of suitable clauses, wherein the plurality of distinct DMRs comprises from about 1 to about 10 distinct DMRs.
33. The method of clause 1, any other suitable clause, or any combination of suitable clauses, wherein the plurality of distinct DMRs comprises from about 5 to about 25 distinct DMRs.
34. The method of clause 1, any other suitable clause, or any combination of suitable clauses, wherein the plurality of distinct DMRs comprises from about 10 to about 50 distinct DMRs.
35. The method of clause 1, any other suitable clause, or any combination of suitable clauses, wherein the plurality of distinct DMRs comprises from about 30 to about 80 distinct DMRs.
36. The method of clause 1, any other suitable clause, or any combination of suitable clauses, wherein the plurality of distinct DMRs comprises from about 50 to about 100 distinct DMRs.
37. The method of clause 1, any other suitable clause, or any combination of suitable clauses, wherein the plurality of distinct DMRs comprises from about 100 to about 150 distinct DMRs.
38. The method of clause 1, any other suitable clause, or any combination of suitable clauses, wherein the plurality of distinct DMRs comprises from about 150 to about 200 distinct DMRs.
39. The method of clause 1, any other suitable clause, or any combination of suitable clauses, wherein the plurality of distinct DMRs comprises from about 200 to about 250 distinct DMRs.
40. The method of clause 1, any other suitable clause, or any combination of suitable clauses, wherein the plurality of distinct DMRs comprises from about 250 to about 300 distinct DMRs.
41. The method of clause 1, any other suitable clause, or any combination of suitable clauses, wherein the plurality of distinct DMRs comprises from about 300 to about 350 distinct DMRs.
42. The method of clause 1, any other suitable clause, or any combination of suitable clauses, wherein the plurality of distinct DMRs comprises from about 350 to about 400 distinct DMRs.
43. The method of clause 1, any other suitable clause, or any combination of suitable clauses, wherein the plurality of distinct DMRs comprises from about 400 to about 450 distinct DMRs.
44. The method of clause 1, any other suitable clause, or any combination of suitable clauses, wherein the plurality of distinct DMRs comprises from about 450 to about 500 distinct DMRs.
45. The method of clause 1, any other suitable clause, or any combination of suitable clauses, wherein the plurality of distinct DMRs comprises from about 500 to about 550 distinct DMRs.
46. The method of clause 1, any other suitable clause, or any combination of suitable clauses, wherein the plurality of distinct DMRs comprises from about 550 to about 600 distinct DMRs.
47. The method of clause 1, any other suitable clause, or any combination of suitable clauses, wherein the plurality of distinct DMRs comprises from about 600 to about 650 distinct DMRs.
48. The method of clause 1, any other suitable clause, or any combination of suitable clauses, wherein the plurality of distinct DMRs comprises from about 650 to about 700 distinct DMRs.
49. The method of clause 1, any other suitable clause, or any combination of suitable clauses, wherein the plurality of distinct DMRs comprises from about 700 to about 750 distinct DMRs.
50. The method of clause 1, any other suitable clause, or any combination of suitable clauses, wherein the plurality of distinct DMRs comprises from about 750 to about 800 distinct DMRs.
51. The method of clause 1, any other suitable clause, or any combination of suitable clauses, wherein the plurality of distinct DMRs comprises from about 800 to about 850 distinct DMRs.
52. The method of clause 1, any other suitable clause, or any combination of suitable clauses, wherein the plurality of distinct DMRs comprises from about 850 to about 900 distinct DMRs.
53. The method of clause 1, any other suitable clause, or any combination of suitable clauses, wherein the plurality of distinct DMRs comprises from about 900 to about 950 distinct DMRs.
54. The method of clause 1, any other suitable clause, or any combination of suitable clauses, wherein the plurality of distinct DMRs comprises from about 950 to about 1000 distinct DMRs.
55. The method of clause 1, any other suitable clause, or any combination of suitable clauses, wherein the plurality of distinct DMRs demonstrate an increase in DNA methylation.
56. The method of clause 1, any other suitable clause, or any combination of suitable clauses, wherein the plurality of distinct DMRs demonstrate a decrease in DNA methylation.
57. The method of clause 1, any other suitable clause, or any combination of suitable clauses, wherein the plurality of distinct DMRs are of a DMR associated gene category.
58. The method of clause 57, any other suitable clause, or any combination of suitable clauses, wherein the DMR associated gene category is a signaling gene category.
59. The method of clause 57, any other suitable clause, or any combination of suitable clauses, wherein the DMR associated gene category is a transcription gene category.
60. The method of clause 57, any other suitable clause, or any combination of suitable clauses, wherein the DMR associated gene category is a metabolism gene category.
61. The method of clause 57, any other suitable clause, or any combination of suitable clauses, wherein the DMR associated gene category is a receptor gene category.
62. The method of clause 57, any other suitable clause, or any combination of suitable clauses, wherein the DMR associated gene category is a cytoskeleton gene category.
63. The method of clause 57, any other suitable clause, or any combination of suitable clauses, wherein the DMR associated gene category is a development gene category.
64. The method of clause 57, any other suitable clause, or any combination of suitable clauses, wherein the DMR associated gene category is a transport gene category.
65. The method of clause 57, any other suitable clause, or any combination of suitable clauses, wherein the DMR associated gene category is an extracellular matrix gene category.
66. The method of clause 57, any other suitable clause, or any combination of suitable clauses, wherein the DMR associated gene category is an epigenetic gene category.
67. The method of clause 57, any other suitable clause, or any combination of suitable clauses, wherein the DMR associated gene category is a protease gene category.
68. The method of clause 57, any other suitable clause, or any combination of suitable clauses, wherein the DMR associated gene category is a proteolysis gene category.
69. The method of clause 57, any other suitable clause, or any combination of suitable clauses, wherein the DMR associated gene category is a translation gene category.
70. The method of clause 57, any other suitable clause, or any combination of suitable clauses, wherein the DMR associated gene category is a binding protein gene category.
71. The method of clause 57, any other suitable clause, or any combination of suitable clauses, wherein the DMR associated gene category is a growth factor/cytokine gene category.
72. The method of clause 57, any other suitable clause, or any combination of suitable clauses, wherein the DMR associated gene category is a cell cycle gene category.
73. The method of clause 57, any other suitable clause, or any combination of suitable clauses, wherein the DMR associated gene category is an immune gene category.
74. The method of clause 57, any other suitable clause, or any combination of suitable clauses, wherein the DMR associated gene category is an apoptosis gene category.
75. The method of clause 57, any other suitable clause, or any combination of suitable clauses, wherein the DMR associated gene category is a Golgi gene category.
76. The method of clause 57, any other suitable clause, or any combination of suitable clauses, wherein the DMR associated gene category is an EST gene category.
77. The method of clause 57, any other suitable clause, or any combination of suitable clauses, wherein the DMR associated gene category is an electron transport gene category. 78. The method of clause 57, any other suitable clause, or any combination of suitable clauses, wherein the DMR associated gene category is a protein binding gene category.
79. The method of clause 57, any other suitable clause, or any combination of suitable clauses, wherein the DMR associated gene category is a DNA repair gene category.
80. The method of clause 1, any other suitable clause, or any combination of suitable clauses, comprising sequencing, wherein the sequencing comprises sequencing by synthesis, ion semiconductor sequencing, single molecule real time sequencing, nanopore sequencing, next-generation sequencing, or any combination thereof.
81. The method of clause 1, any other suitable clause, or any combination of suitable clauses, wherein the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.
82. The method of clause 1, any other suitable clause, or any combination of suitable clauses, wherein the DMRs that are determined and compared, individually, range from about 100 to about 17000 adjacent nucleotides.
83. The method of clause 1, any other suitable clause, or any combination of suitable clauses, wherein each DMR in a plurality of the DMRs that are determined and compared independently comprise a CpG density of about 0.1 to about 11 CpG per 100 nucleotides.
84. The method of clause 83, any other suitable clause, or any combination of suitable clauses, wherein each DMR in a plurality of the DMRs that are determined and compared independently comprise a CpG density of about 1 to about 10 CpG per 100 nucleotides.
85. The method of clause 83, any other suitable clause, or any combination of suitable clauses, wherein each DMR in a plurality of the DMRs that are determined and compared independently comprise a CpG density of less than about 11 CpG per 100 nucleotides.
86. The method of clause 83, any other suitable clause, or any combination of suitable clauses, wherein each DMR in a plurality of the DMRs that are determined and compared independently comprise a CpG density of less than about 5 CpG per 100 nucleotides.
87. The method of clause 83, any other suitable clause, or any combination of suitable clauses, wherein each DMR in a plurality of the DMRs that are determined and compared independently comprise a CpG density of less than about 3 CpG per 100 nucleotides.
88. The method of clause 83, any other suitable clause, or any combination of suitable clauses, wherein each DMR in a plurality of the DMRs that are determined and compared independently comprise a CpG density of between 1 CpG and 3 CpG per 100 nucleotides.
89. The method of clause 1, any other suitable clause, or any combination of suitable clauses, wherein at least about 10, 20, 30, 40, 50, 60, 70, 80, or 90 percent of the DMRs that are determined and compared are hypermethylated when compared, individually, to individual reference methylation levels of corresponding individual reference DMRs.
90. The method of clause 1, any other suitable clause, or any combination of suitable clauses, wherein at least about 30, 40, or 50 percent of the DMRs that are determined and compared are hypomethylated when compared, individually, to individual reference methylation levels of corresponding individual reference DMRs.
91. The method of clause 1, any other suitable clause, or any combination of suitable clauses, wherein all of the DMRs that are determined and compared are hypomethylated when compared, individually, to individual reference methylation levels of corresponding individual reference DMRs.
92. The method of clause 1, any other suitable clause, or any combination of suitable clauses, wherein all of the DMRs that are determined and compared are hypermethylated when compared, individually, to individual reference methylation levels of corresponding individual reference DMRs.
93. The method of clause 1, any other suitable clause, or any combination of suitable clauses, wherein the human subject is a male subject.
94. The method of clause 1, any other suitable clause, or any combination of suitable clauses, wherein the human subject is a female subject.
95. The method of clause 1, any other suitable clause, or any combination of suitable clauses, wherein the human subject is a female infant.
96. The method of clause 1, any other suitable clause, or any combination of suitable clauses, wherein the human subject is a male infant.
97. The method of clause 1, any other suitable clause, or any combination of suitable clauses, wherein the method further comprises determining a risk of the human subject for preterm birth, wherein the determining is performed with a computer comprising a computer processor and computer readable memory comprising computer readable instructions.
98. The method of clause 1, any other suitable clause, or any combination of suitable clauses, further comprising performing an additional analysis using a computer.
99. The method of clause 98, any other suitable clause, or any combination of suitable clauses, wherein the additional analysis comprises a principle component analysis (PCA), a dendrogram analysis, a machine learning analysis, or any combination thereof.
100. The method of clause 98, any other suitable clause, or any combination of suitable clauses, wherein the additional analysis generates data points, and wherein the data points in the additional analysis are grouped into two spatially distinct categories comprising a first category and a second category.
101. The method of clause 100, any other suitable clause, or any combination of suitable clauses, wherein the first category indicates the human subject is at increased risk of having preterm birth and wherein the second category indicates the human subject is not at increased risk of having preterm birth.
102. The method of clause 1, any other suitable clause, or any combination of suitable clauses, further comprising treating the human subject.
103. The method of clause 102, any other suitable clause, or any combination of suitable clauses, wherein the treating comprises administering clinical management for prevention of preterm birth.
104. The method of clause 103, any other suitable clause, or any combination of suitable clauses, wherein the clinical management comprises enhanced monitoring of pregnancy.
105. The method of clause 103, any other suitable clause, or any combination of suitable clauses, wherein the clinical management comprises application of protective uterine monitoring.
106. The method of clause 103, any other suitable clause, or any combination of suitable clauses, wherein the clinical management comprises hospital admission of the human subject.
107. The method of clause 103, any other suitable clause, or any combination of suitable clauses, wherein the clinical management comprises operative delivery performed on the human subject.
108. The method of clause 102, any other suitable clause, or any combination of suitable clauses, wherein the treating comprises administering a medication for preventative treatment of preterm birth.
109. The method of clause 108, any other suitable clause, or any combination of suitable clauses, wherein the medication is a prenatal steroid.
110. The method of clause 108, any other suitable clause, or any combination of suitable clauses, wherein the medication is nitroglycerine.
111. The method of clause 108, any other suitable clause, or any combination of suitable clauses, wherein the medication is a tocolytic.
112. The method of clause 111, any other suitable clause, or any combination of suitable clauses, wherein the tocolytic is an oxytocin receptor (OTR) antagonist.
113. The method of clause 111, any other suitable clause, or any combination of suitable clauses, wherein the tocolytic is selected from the group consisting of a β2 agonist, a calcium channel blocker, a nonsteroidal anti-inflammatory drug (NSAID), magnesium sulfate, and any combination thereof.
114. The method of clause 111, any other suitable clause, or any combination of suitable clauses, wherein the tocolytic is a beta-adrenergic receptor agonist.
115. The method of clause 114, any other suitable clause, or any combination of suitable clauses, wherein the beta-adrenergic receptor agonist is selected from the group consisting of terbutaline, hexoprenaline, ritodrine, a salt of any of these, or any combination thereof.
116. The method of clause 111, any other suitable clause, or any combination of suitable clauses, wherein the tocolytic is a calcium channel blocker.
117. The method of clause 116, any other suitable clause, or any combination of suitable clauses, wherein the calcium channel blocker is nifedipine or a salt thereof.
118. The method of clause 111, any other suitable clause, or any combination of suitable clauses, wherein the tocolytic is an NSAID.
119. The method of clause 118, any other suitable clause, or any combination of suitable clauses, wherein the NSAID is indomethacin or a salt thereof.
120. The method of clause 111, any other suitable clause, or any combination of suitable clauses, wherein the tocolytic is magnesium sulfate.
121. The method of clause 111, any other suitable clause, or any combination of suitable clauses, wherein the tocolytic is an oxytocin inhibitor.
122. The method of clause 121, any other suitable clause, or any combination of suitable clauses, wherein the oxytocin inhibitor is atosiban, retosiban, a salt of any of these, or any combination thereof.
123. The method of clause 111, any other suitable clause, or any combination of suitable clauses, wherein the tocolytic is terbutaline.
124. The method of clause 111, any other suitable clause, or any combination of suitable clauses, wherein the tocolytic is ritodrine.
125. The method of clause 111, any other suitable clause, or any combination of suitable clauses, wherein the tocolytic is fenoterol.
126. The method of clause 111, any other suitable clause, or any combination of suitable clauses, wherein the tocolytic is albuterol.
127. The method of clause 111, any other suitable clause, or any combination of suitable clauses, wherein the tocolytic is hexoprenaline.
128. The method of clause 111, any other suitable clause, or any combination of suitable clauses, wherein the tocolytic is nifedipine.
129. The method of clause 111, any other suitable clause, or any combination of suitable clauses, wherein the tocolytic is atosiban.
130. The method of clause 111, any other suitable clause, or any combination of suitable clauses, wherein the tocolytic is indomethacin.
131. The method of clause 111, any other suitable clause, or any combination of suitable clauses, wherein the tocolytic is sulindac.
132. The method of clause 1, any other suitable clause, or any combination of suitable clauses, further comprising transmitting data, a result, or both, via an electronic communication medium.
133. A method comprising:
   obtaining a sample of a somatic cell from a human subject;
   isolating deoxyribonucleic acid (DNA) from the sample, thereby obtaining isolated DNA;
   detecting and determining a methylation level of a differential DNA methylation region (DMR) comprised in the isolated DNA; and
   comparing the methylation level of the DMR to a reference methylation level of a corresponding reference DMR;
   wherein the comparing comprises employing a computer comprising a computer processor and computer readable memory comprising computer readable instructions contained thereon,
   wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
   wherein a plurality of determined DMRs are sufficient to determine, from a process comprising the comparing and employing a computer, whether the human subject has or is at increased risk of preterm birth.
134. The method of clause 133, any other suitable clause, or any combination of suitable clauses, wherein the somatic cell comprises a buccal cell.
135. The method of clause 133, any other suitable clause, or any combination of suitable clauses, wherein the somatic cell consists essentially of a buccal cell.
136. The method of clause 133, any other suitable clause, or any combination of suitable clauses, wherein the somatic cell consists of a buccal cell.
137. The method of clause 133, any other suitable clause, or any combination of suitable clauses, wherein the somatic cell comprises a blood monocyte cell.
138. The method of clause 137, any other suitable clause, or any combination of suitable clauses, wherein the blood monocyte cell is a purified blood monocyte cell.
139. The method of clause 133, any other suitable clause, or any combination of suitable clauses, wherein the somatic cell consists essentially of a blood monocyte cell.
140. The method of clause 139, any other suitable clause, or any combination of suitable clauses, wherein the blood monocyte cell is a purified blood monocyte cell.
141. The method of clause 133, any other suitable clause, or any combination of suitable clauses, wherein the somatic cell consists of a blood monocyte cell.
142. The method of clause 141, any other suitable clause, or any combination of suitable clauses, wherein the blood monocyte cell is a purified blood monocyte cell.
143. The method of clause 133, any other suitable clause, or any combination of suitable clauses, wherein the somatic cell comprises a muscle cell, a blood cell, a skin cell, a nerve cell, or any combination thereof.
144. The method of clause 133, any other suitable clause, or any combination of suitable clauses, wherein the somatic cell comprises an immune cell.
145. The method of clause 133, any other suitable clause, or any combination of suitable clauses, wherein the somatic cell comprises a muscle cell.
146. The method of clause 133, any other suitable clause, or any combination of suitable clauses, wherein the somatic cell comprises a blood cell.
147. The method of clause 133, any other suitable clause, or any combination of suitable clauses, wherein the somatic cell comprises a skin cell.
148. The method of clause 133, any other suitable clause, or any combination of suitable clauses, wherein the somatic cell comprises a nerve cell.
149. The method of clause 133, any other suitable clause, or any combination of suitable clauses, wherein the plurality of determined DMRs are selected from the DMRs in Table 1, the DMRs in Table 2, the DMRs in Table 3, the DMRs in Table 4, and any combination thereof.
150. The method of clause 133, any other suitable clause, or any combination of suitable clauses, wherein the plurality of determined DMRs are selected from the DMRs in Table 1.
151. The method of clause 133, any other suitable clause, or any combination of suitable clauses, wherein the plurality of determined DMRs are selected from the DMRs in Table 2.
152. The method of clause 133, any other suitable clause, or any combination of suitable clauses, wherein the plurality of determined DMRs are selected from the DMRs in Table 3.
153. The method of clause 133, any other suitable clause, or any combination of suitable clauses, wherein the plurality of determined DMRs are selected from the DMRs in Table 4.
154. The method of clause 133, any other suitable clause, or any combination of suitable clauses, wherein the plurality of distinct DMRs comprises at least 10 distinct DMRs.
155. The method of clause 133, any other suitable clause, or any combination of suitable clauses, wherein the plurality of distinct DMRs comprises 2 to about 10 distinct DMRs.
156. The method of clause 133, any other suitable clause, or any combination of suitable clauses, wherein the plurality of distinct DMRs comprises 5 to about 15 distinct DMRs.
157. The method of clause 133, any other suitable clause, or any combination of suitable clauses, wherein the plurality of distinct DMRs comprises 3 to about 8 distinct DMRs.
158. The method of clause 133, any other suitable clause, or any combination of suitable clauses, wherein the plurality of distinct DMRs comprises 4 to about 12 distinct DMRs.
159. The method of clause 133, any other suitable clause, or any combination of suitable clauses, wherein the plurality of distinct DMRs comprises about 20 to about 30 distinct DMRs.
160. The method of clause 133, any other suitable clause, or any combination of suitable clauses, wherein the plurality of distinct DMRs comprises about 10 to about 1000 distinct DMRs.
161. The method of clause 133, any other suitable clause, or any combination of suitable clauses, wherein the plurality of distinct DMRs comprises about 10 to about 15 distinct DMRs.
162. The method of clause 133, any other suitable clause, or any combination of suitable clauses, wherein the plurality of distinct DMRs comprises about 10 to about 50 distinct DMRs.
163. The method of clause 133, any other suitable clause, or any combination of suitable clauses, wherein the plurality of distinct DMRs comprises about 10 to about 100 distinct DMRs.
164. The method of clause 133, any other suitable clause, or any combination of suitable clauses, wherein the plurality of distinct DMRs comprises from about 1 to about 10 distinct DMRs.
165. The method of clause 133, any other suitable clause, or any combination of suitable clauses, wherein the plurality of distinct DMRs comprises from about 5 to about 25 distinct DMRs.
166. The method of clause 133, any other suitable clause, or any combination of suitable clauses, wherein the plurality of distinct DMRs comprises from about 10 to about 50 distinct DMRs.
167. The method of clause 133, any other suitable clause, or any combination of suitable clauses, wherein the plurality of distinct DMRs comprises from about 30 to about 80 distinct DMRs.
168. The method of clause 133, any other suitable clause, or any combination of suitable clauses, wherein the plurality of determined DMRs comprises about 50 to about 100 DMRs.
169. The method of clause 133, any other suitable clause, or any combination of suitable clauses, wherein the plurality of determined DMRs comprises about 100 to about 150 DMRs.
170. The method of clause 133, any other suitable clause, or any combination of suitable clauses, wherein the plurality of determined DMRs comprises about 150 to about 200 DMRs.
171. The method of clause 133, any other suitable clause, or any combination of suitable clauses, wherein the plurality of determined DMRs comprises about 200 to about 250 DMRs.
172. The method of clause 133, any other suitable clause, or any combination of suitable clauses, wherein the plurality of determined DMRs comprises about 250 to about 300 DMRs.
173. The method of clause 133, any other suitable clause, or any combination of suitable clauses, wherein the plurality of determined DMRs comprises about 300 to about 350 DMRs.
174. The method of clause 133, any other suitable clause, or any combination of suitable clauses, wherein the plurality of determined DMRs comprises about 350 to about 400 DMRs.
175. The method of clause 133, any other suitable clause, or any combination of suitable clauses, wherein the plurality of determined DMRs comprises about 400 to about 450 DMRs.
176. The method of clause 133, any other suitable clause, or any combination of suitable clauses, wherein the plurality of determined DMRs comprises about 450 to about 500 DMRs.
177. The method of clause 133, any other suitable clause, or any combination of suitable clauses, wherein the plurality of determined DMRs comprises about 500 to about 550 DMRs.
178. The method of clause 133, any other suitable clause, or any combination of suitable clauses, wherein the plurality of determined DMRs comprises about 550 to about 600 DMRs.
179. The method of clause 133, any other suitable clause, or any combination of suitable clauses, wherein the plurality of determined DMRs comprises about 600 to about 650 DMRs.
180. The method of clause 133, any other suitable clause, or any combination of suitable clauses, wherein the plurality of determined DMRs comprises about 650 to about 700 DMRs.
181. The method of clause 133, any other suitable clause, or any combination of suitable clauses, wherein the plurality of determined DMRs comprises about 700 to about 750 DMRs.
182. The method of clause 133, any other suitable clause, or any combination of suitable clauses, wherein the plurality of determined DMRs comprises about 750 to about 800 DMRs.
183. The method of clause 133, any other suitable clause, or any combination of suitable clauses, wherein the plurality of determined DMRs comprises about 800 to about 850 DMRs.
184. The method of clause 133, any other suitable clause, or any combination of suitable clauses, wherein the plurality of determined DMRs comprises about 850 to about 900 DMRs.
185. The method of clause 133, any other suitable clause, or any combination of suitable clauses, wherein the plurality of determined DMRs comprises about 900 to about 950 DMRs.
186. The method of clause 133, any other suitable clause, or any combination of suitable clauses, wherein the plurality of determined DMRs comprises about 950 to about 1000 DMRs.
187. The method of clause 133, any other suitable clause, or any combination of suitable clauses, wherein the plurality of distinct DMRs demonstrate an increase in DNA methylation.
188. The method of clause 133, any other suitable clause, or any combination of suitable clauses, wherein the plurality of distinct DMRs demonstrate a decrease in DNA methylation.
189. The method of clause 133, any other suitable clause, or any combination of suitable clauses, wherein the plurality of distinct DMRs are of a DMR associated gene category.
190. The method of clause 189, any other suitable clause, or any combination of suitable clauses, wherein the DMR associated gene category is a signaling gene category.
191. The method of clause 189, any other suitable clause, or any combination of suitable clauses, wherein the DMR associated gene category is a transcription gene category.
192. The method of clause 189, any other suitable clause, or any combination of suitable clauses, wherein the DMR associated gene category is a metabolism gene category.
193. The method of clause 189, any other suitable clause, or any combination of suitable clauses, wherein the DMR associated gene category is a receptor gene category.
194. The method of clause 189, any other suitable clause, or any combination of suitable clauses, wherein the DMR associated gene category is a cytoskeleton gene category.
195. The method of clause 189, any other suitable clause, or any combination of suitable clauses, wherein the DMR associated gene category is a development gene category.
196. The method of clause 189, any other suitable clause, or any combination of suitable clauses, wherein the DMR associated gene category is a transport gene category.
197. The method of clause 189, any other suitable clause, or any combination of suitable clauses, wherein the DMR associated gene category is an extracellular matrix gene category.
198. The method of clause 189, any other suitable clause, or any combination of suitable clauses, wherein the DMR associated gene category is an epigenetic gene category.
199. The method of clause 189, any other suitable clause, or any combination of suitable clauses, wherein the DMR associated gene category is a protease gene category.
200. The method of clause 189, any other suitable clause, or any combination of suitable clauses, wherein the DMR associated gene category is a proteolysis gene category.
201. The method of clause 189, any other suitable clause, or any combination of suitable clauses, wherein the DMR associated gene category is a translation gene category.
202. The method of clause 189, any other suitable clause, or any combination of suitable clauses, wherein the DMR associated gene category is a binding protein gene category.
203. The method of clause 189, any other suitable clause, or any combination of suitable clauses, wherein the DMR associated gene category is a growth factor/cytokine gene category.
204. The method of clause 189, any other suitable clause, or any combination of suitable clauses, wherein the DMR associated gene category is a cell cycle gene category.
205. The method of clause 189, any other suitable clause, or any combination of suitable clauses, wherein the DMR associated gene category is an immune gene category.
206. The method of clause 189, any other suitable clause, or any combination of suitable clauses, wherein the DMR associated gene category is an apoptosis gene category.
207. The method of clause 189, any other suitable clause, or any combination of suitable clauses, wherein the DMR associated gene category is a Golgi gene category.
208. The method of clause 189, any other suitable clause, or any combination of suitable clauses, wherein the DMR associated gene category is an EST gene category.
209. The method of clause 189, any other suitable clause, or any combination of suitable clauses, wherein the DMR associated gene category is an electron transport gene category.
210. The method of clause 189, any other suitable clause, or any combination of suitable clauses, wherein the DMR associated gene category is a protein binding gene category.
211. The method of clause 133, any other suitable clause, or any combination of suitable clauses, wherein the DMR associated gene category is a DNA repair gene category.
212. The method of clause 133, any other suitable clause, or any combination of suitable clauses, wherein the human subject is a male subject.
213. The method of clause 133, any other suitable clause, or any combination of suitable clauses, wherein the human subject is a female subject.
214. The method of clause 133, any other suitable clause, or any combination of suitable clauses, wherein the human subject is a female infant.
215. The method of clause 133, any other suitable clause, or any combination of suitable clauses, wherein the human subject is a male infant.
216. The method of clause 133, any other suitable clause, or any combination of suitable clauses, further comprising treating the human subject.
217. The method of clause 216, any other suitable clause, or any combination of suitable clauses, wherein the treating comprises administering clinical management for prevention of preterm birth.
218. The method of clause 217, any other suitable clause, or any combination of suitable clauses, wherein the clinical management comprises enhanced monitoring of pregnancy.
219. The method of clause 217, any other suitable clause, or any combination of suitable clauses, wherein the clinical management comprises application of protective uterine monitoring.
220. The method of clause 217, any other suitable clause, or any combination of suitable clauses, wherein the clinical management comprises hospital admission of the human subject.
221. The method of clause 217, any other suitable clause, or any combination of suitable clauses, wherein the clinical management comprises operative delivery performed on the human subject.
222. The method of clause 216, any other suitable clause, or any combination of suitable clauses, wherein the treating comprises administering a medication for preventative treatment of preterm birth.
223. The method of clause 222, any other suitable clause, or any combination of suitable clauses, wherein the medication is a prenatal steroid.
224. The method of clause 222, any other suitable clause, or any combination of suitable clauses, wherein the medication is nitroglycerine.
225. The method of clause 222, any other suitable clause, or any combination of suitable clauses, wherein the medication is a tocolytic.
226. The method of clause 225, any other suitable clause, or any combination of suitable clauses, wherein the tocolytic is an oxytocin receptor (OTR) antagonist.
227. The method of clause 225, any other suitable clause, or any combination of suitable clauses, wherein the tocolytic is selected from the group consisting of a β2 agonist, a calcium channel blocker, a nonsteroidal anti-inflammatory drug (NSAID), magnesium sulfate, and any combination thereof.
228. The method of clause 225, any other suitable clause, or any combination of suitable clauses, wherein the tocolytic is a beta-adrenergic receptor agonist.
229. The method of clause 228, any other suitable clause, or any combination of suitable clauses, wherein the beta-adrenergic receptor agonist is selected from the group consisting of terbutaline, hexoprenaline, ritodrine, a salt of any of these, or any combination thereof.
230. The method of clause 225, any other suitable clause, or any combination of suitable clauses, wherein the tocolytic is a calcium channel blocker.
231. The method of clause 230, any other suitable clause, or any combination of suitable clauses, wherein the calcium channel blocker is nifedipine or a salt thereof.
232. The method of clause 225, any other suitable clause, or any combination of suitable clauses, wherein the tocolytic is an NSAID.
233. The method of clause 232, any other suitable clause, or any combination of suitable clauses, wherein the NSAID is indomethacin or a salt thereof.
234. The method of clause 225, any other suitable clause, or any combination of suitable clauses, wherein the tocolytic is magnesium sulfate.
235. The method of clause 225, any other suitable clause, or any combination of suitable clauses, wherein the tocolytic is an oxytocin inhibitor.
236. The method of clause 235, any other suitable clause, or any combination of suitable clauses, wherein the oxytocin inhibitor is atosiban, retosiban, a salt of any of these, or any combination thereof.
237. The method of clause 225, any other suitable clause, or any combination of suitable clauses, wherein the tocolytic is terbutaline.
238. The method of clause 225, any other suitable clause, or any combination of suitable clauses, wherein the tocolytic is ritodrine.
239. The method of clause 225, any other suitable clause, or any combination of suitable clauses, wherein the tocolytic is fenoterol.
240. The method of clause 225, any other suitable clause, or any combination of suitable clauses, wherein the tocolytic is albuterol.
241. The method of clause 225, any other suitable clause, or any combination of suitable clauses, wherein the tocolytic is hexoprenaline.
242. The method of clause 225, any other suitable clause, or any combination of suitable clauses, wherein the tocolytic is nifedipine.
243. The method of clause 225, any other suitable clause, or any combination of suitable clauses, wherein the tocolytic is atosiban.
244. The method of clause 225, any other suitable clause, or any combination of suitable clauses, wherein the tocolytic is indomethacin.
245. The method of clause 225, any other suitable clause, or any combination of suitable clauses, wherein the tocolytic is sulindac.
246. The method of clause 133, any other suitable clause, or any combination of suitable clauses, further comprising transmitting data, a result, or both, via an electronic communication medium.
247. A kit comprising at least about 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence selected from the DMRs in Table 1, the DMRs in Table 2, the DMRs in Table 3, the DMRs in Table 4, and any combination thereof; and a container.
248. The kit of clause 247, any other suitable clause, or any combination of suitable clauses, wherein the distinct primers or pairs of primers each further comprise a unique barcode.
249. The kit of clause 247, any other suitable clause, or any combination of suitable clauses, wherein the distinct primers or pairs of primers are not bound to an array or a microarray.
250. The kit of clause 247, any other suitable clause, or any combination of suitable clauses, wherein the distinct primers or pairs of primers are bound to an array or a microarray.
251. The kit of clause 247, any other suitable clause, or any combination of suitable clauses, wherein the distinct primers or pairs of primers comprise DNA.
252. The kit of clause 247, any other suitable clause, or any combination of suitable clauses, wherein the distinct DMR sequences are selected from the DMRs in Table 1.
253. The kit of clause 247, any other suitable clause, or any combination of suitable clauses, wherein the distinct DMR sequences are selected from the DMRs in Table 2.
254. The kit of clause 247, any other suitable clause, or any combination of suitable clauses, wherein the distinct DMR sequences are selected from the DMRs in Table 3.
255. The kit of clause 247, any other suitable clause, or any combination of suitable clauses, wherein the distinct DMR sequences are selected from the DMRs in Table 4.
256. The kit of clause 247, any other suitable clause, or any combination of suitable clauses, wherein the distinct DMR sequences comprises about 1 to about 10 DMRs.
257. The kit of clause 247, any other suitable clause, or any combination of suitable clauses, wherein the distinct DMR sequences comprises about 5 to about 25 DMRs.
258. The kit of clause 247, any other suitable clause, or any combination of suitable clauses, wherein the distinct DMR sequences comprises about 10 to about 50 DMRs.
259. The kit of clause 247, any other suitable clause, or any combination of suitable clauses, wherein the distinct DMR sequences comprises about 30 to about 80 DMRs.
260. The kit of clause 247, any other suitable clause, or any combination of suitable clauses, wherein the distinct DMR sequences comprises about 50 to about 100 DMRs.
261. The kit of clause 247, any other suitable clause, or any combination of suitable clauses, wherein the distinct DMR sequences comprises about 100 to about 150 DMRs.
262. The kit of clause 247, any other suitable clause, or any combination of suitable clauses, wherein the distinct DMR sequences comprises about 150 to about 200 DMRs.
263. The kit of clause 247, any other suitable clause, or any combination of suitable clauses, wherein the distinct DMR sequences comprises about 200 to about 250 DMRs.
264. The kit of clause 247, any other suitable clause, or any combination of suitable clauses, wherein the distinct DMR sequences comprises about 250 to about 300 DMRs.
265. The kit of clause 247, any other suitable clause, or any combination of suitable clauses, wherein the distinct DMR sequences comprises about 300 to about 350 DMRs.
266. The kit of clause 247, any other suitable clause, or any combination of suitable clauses, wherein the distinct DMR sequences comprises about 350 to about 400 DMRs.
267. The kit of clause 247, any other suitable clause, or any combination of suitable clauses, wherein the distinct DMR sequences comprises about 400 to about 450 DMRs.
268. The kit of clause 247, any other suitable clause, or any combination of suitable clauses, wherein the distinct DMR sequences comprises about 450 to about 500 DMRs.
269. The kit of clause 247, any other suitable clause, or any combination of suitable clauses, wherein the distinct DMR sequences comprises about 500 to about 550 DMRs.
270. The kit of clause 247, any other suitable clause, or any combination of suitable clauses, wherein the distinct DMR sequences comprises about 550 to about 600 DMRs.
271. The kit of clause 247, any other suitable clause, or any combination of suitable clauses, wherein the distinct DMR sequences comprises about 600 to about 650 DMRs.
272. The kit of clause 247, any other suitable clause, or any combination of suitable clauses, wherein the distinct DMR sequences comprises about 650 to about 700 DMRs.
273. The kit of clause 247, any other suitable clause, or any combination of suitable clauses, wherein the distinct DMR sequences comprises about 700 to about 750 DMRs.
274. The kit of clause 247, any other suitable clause, or any combination of suitable clauses, wherein the distinct DMR sequences comprises about 750 to about 800 DMRs.
275. The kit of clause 247, any other suitable clause, or any combination of suitable clauses, wherein the distinct DMR sequences comprises about 800 to about 850 DMRs.
276. The kit of clause 247, any other suitable clause, or any combination of suitable clauses, wherein the distinct DMR sequences comprises about 850 to about 900 DMRs.
277. The kit of clause 247, any other suitable clause, or any combination of suitable clauses, wherein the distinct DMR sequences comprises about 900 to about 950 DMRs.
278. The kit of clause 247, any other suitable clause, or any combination of suitable clauses, wherein the distinct DMR sequences comprises about 950 to about 1000 DMRs.
279. The kit of clause 247, any other suitable clause, or any combination of suitable clauses, wherein the distinct DMR sequences are of a DMR associated gene category.
280. The kit of clause 279, any other suitable clause, or any combination of suitable clauses, wherein the DMR associated gene category is a signaling gene category.
281. The kit of clause 279, any other suitable clause, or any combination of suitable clauses, wherein the DMR associated gene category is a transcription gene category.
282. The kit of clause 279, any other suitable clause, or any combination of suitable clauses, wherein the DMR associated gene category is a metabolism gene category.
283. The kit of clause 279, any other suitable clause, or any combination of suitable clauses, wherein the DMR associated gene category is a receptor gene category.
284. The kit of clause 279, any other suitable clause, or any combination of suitable clauses, wherein the DMR associated gene category is a cytoskeleton gene category.
285. The kit of clause 279, any other suitable clause, or any combination of suitable clauses, wherein the DMR associated gene category is a development gene category.
286. The kit of clause 279, any other suitable clause, or any combination of suitable clauses, wherein the DMR associated gene category is a transport gene category.
287. The kit of clause 279, any other suitable clause, or any combination of suitable clauses, wherein the DMR associated gene category is an extracellular matrix gene category.
288. The kit of clause 279, any other suitable clause, or any combination of suitable clauses, wherein the DMR associated gene category is an epigenetic gene category.
289. The kit of clause 279, any other suitable clause, or any combination of suitable clauses, wherein the DMR associated gene category is a protease gene category.
290. The kit of clause 279, any other suitable clause, or any combination of suitable clauses, wherein the DMR associated gene category is a proteolysis gene category.
291. The kit of clause 279, any other suitable clause, or any combination of suitable clauses, wherein the DMR associated gene category is a translation gene category.
292. The kit of clause 279, any other suitable clause, or any combination of suitable clauses, wherein the DMR associated gene category is a binding protein gene category.
293. The kit of clause 279, any other suitable clause, or any combination of suitable clauses, wherein the DMR associated gene category is a growth factor/cytokine gene category.
294. The kit of clause 279, any other suitable clause, or any combination of suitable clauses, wherein the DMR associated gene category is a cell cycle gene category.
295. The kit of clause 279, any other suitable clause, or any combination of suitable clauses, wherein the DMR associated gene category is an immune gene category.
296. The kit of clause 279, any other suitable clause, or any combination of suitable clauses, wherein the DMR associated gene category is an apoptosis gene category.
297. The kit of clause 279, any other suitable clause, or any combination of suitable clauses, wherein the DMR associated gene category is a Golgi gene category.
298. The kit of clause 279, any other suitable clause, or any combination of suitable clauses, wherein the DMR associated gene category is an EST gene category.
299. The kit of clause 279, any other suitable clause, or any combination of suitable clauses, wherein the DMR associated gene category is an electron transport gene category.
300. The kit of clause 279, any other suitable clause, or any combination of suitable clauses, wherein the DMR associated gene category is a protein binding gene category.
301. The kit of clause 279, any other suitable clause, or any combination of suitable clauses, wherein the DMR associated gene category is a DNA repair gene category.
302. A kit comprising at least about 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct probes, each distinct probe complementary to a distinct DMR sequence selected from the DMRs in Table 1, the DMRs in Table 2, the DMRs in Table 3, the DMRs in Table 4, and any combination thereof; and a container.
303. The kit of clause 302, any other suitable clause, or any combination of suitable clauses, wherein the distinct probes further comprises at least one of fluorophore, a chromophore, a barcode, or any combination thereof.
304. The kit of clause 303, any other suitable clause, or any combination of suitable clauses, wherein each probe comprises a unique fluorophore, a chromophore, barcode, or any combination thereof.
305. The kit of clause 302, any other suitable clause, or any combination of suitable clauses, wherein the distinct primers or pairs of primers are not bound to an array or a microarray.
306. The kit of clause 302, any other suitable clause, or any combination of suitable clauses, wherein the distinct primers or pairs of primers are bound to an array or a microarray.
307. The kit of clause 302, any other suitable clause, or any combination of suitable clauses, wherein the distinct primers or pairs of primers comprise DNA.
308. The kit of clause 302, any other suitable clause, or any combination of suitable clauses, wherein the distinct DMR sequences are selected from the DMRs in Table 1.
309. The kit of clause 302, any other suitable clause, or any combination of suitable clauses, wherein the distinct DMR sequences are selected from the DMRs in Table 2.
310. The kit of clause 302, any other suitable clause, or any combination of suitable clauses, wherein the distinct DMR sequences are selected from the DMRs in Table 3.
311. The kit of clause 302, any other suitable clause, or any combination of suitable clauses, wherein the distinct DMR sequences are selected from the DMRs in Table 4.
312. The kit of clause 302, any other suitable clause, or any combination of suitable clauses, wherein the distinct DMR sequences comprises about 1 to about 10 DMRs.
313. The kit of clause 302, any other suitable clause, or any combination of suitable clauses, wherein the distinct DMR sequences comprises about 5 to about 25 DMRs.
314. The kit of clause 302, any other suitable clause, or any combination of suitable clauses, wherein the distinct DMR sequences comprises about 10 to about 50 DMRs.
315. The kit of clause 302, any other suitable clause, or any combination of suitable clauses, wherein the distinct DMR sequences comprises about 30 to about 80 DMRs.
316. The kit of clause 302, any other suitable clause, or any combination of suitable clauses, wherein the distinct DMR sequences comprises about 50 to about 100 DMRs.
317. The kit of clause 302, any other suitable clause, or any combination of suitable clauses, wherein the distinct DMR sequences comprises about 100 to about 150 DMRs.
318. The kit of clause 302, any other suitable clause, or any combination of suitable clauses, wherein the distinct DMR sequences comprises about 150 to about 200 DMRs.
319. The kit of clause 302, any other suitable clause, or any combination of suitable clauses, wherein the distinct DMR sequences comprises about 200 to about 250 DMRs.
320. The kit of clause 302, any other suitable clause, or any combination of suitable clauses, wherein the distinct DMR sequences comprises about 250 to about 300 DMRs.
321. The kit of clause 302, any other suitable clause, or any combination of suitable clauses, wherein the distinct DMR sequences comprises about 300 to about 350 DMRs.
322. The kit of clause 302, any other suitable clause, or any combination of suitable clauses, wherein the distinct DMR sequences comprises about 350 to about 400 DMRs.
323. The kit of clause 302, any other suitable clause, or any combination of suitable clauses, wherein the distinct DMR sequences comprises about 400 to about 450 DMRs.
324. The kit of clause 302, any other suitable clause, or any combination of suitable clauses, wherein the distinct DMR sequences comprises about 450 to about 500 DMRs.
325. The kit of clause 302, any other suitable clause, or any combination of suitable clauses, wherein the distinct DMR sequences comprises about 500 to about 550 DMRs.
326. The kit of clause 302, any other suitable clause, or any combination of suitable clauses, wherein the distinct DMR sequences comprises about 550 to about 600 DMRs.
327. The kit of clause 302, any other suitable clause, or any combination of suitable clauses, wherein the distinct DMR sequences comprises about 600 to about 650 DMRs.
328. The kit of clause 302, any other suitable clause, or any combination of suitable clauses, wherein the distinct DMR sequences comprises about 650 to about 700 DMRs.
329. The kit of clause 302, any other suitable clause, or any combination of suitable clauses, wherein the distinct DMR sequences comprises about 700 to about 750 DMRs.
330. The kit of clause 302, any other suitable clause, or any combination of suitable clauses, wherein the distinct DMR sequences comprises about 750 to about 800 DMRs.
331. The kit of clause 302, any other suitable clause, or any combination of suitable clauses, wherein the distinct DMR sequences comprises about 800 to about 850 DMRs.
332. The kit of clause 302, any other suitable clause, or any combination of suitable clauses, wherein the distinct DMR sequences comprises about 850 to about 900 DMRs.
333. The kit of clause 302, any other suitable clause, or any combination of suitable clauses, wherein the distinct DMR sequences comprises about 900 to about 950 DMRs.
334. The kit of clause 302, any other suitable clause, or any combination of suitable clauses, wherein the distinct DMR sequences comprises about 950 to about 1000 DMRs.
335. The kit of clause 302, any other suitable clause, or any combination of suitable clauses, wherein the distinct DMR sequences are of a DMR associated gene category.
336. The kit of clause 335, any other suitable clause, or any combination of suitable clauses, wherein the DMR associated gene category is a signaling gene category.
337. The kit of clause 335, any other suitable clause, or any combination of suitable clauses, wherein the DMR associated gene category is a transcription gene category.
338. The kit of clause 335, any other suitable clause, or any combination of suitable clauses, wherein the DMR associated gene category is a metabolism gene category.
339. The kit of clause 335, any other suitable clause, or any combination of suitable clauses, wherein the DMR associated gene category is a receptor gene category.
340. The kit of clause 335, any other suitable clause, or any combination of suitable clauses, wherein the DMR associated gene category is a cytoskeleton gene category.
341. The kit of clause 335, any other suitable clause, or any combination of suitable clauses, wherein the DMR associated gene category is a development gene category.
342. The kit of clause 335, any other suitable clause, or any combination of suitable clauses, wherein the DMR associated gene category is a transport gene category.
343. The kit of clause 335, any other suitable clause, or any combination of suitable clauses, wherein the DMR associated gene category is an extracellular matrix gene category.
344. The kit of clause 335, any other suitable clause, or any combination of suitable clauses, wherein the DMR associated gene category is an epigenetic gene category.
345. The kit of clause 335, any other suitable clause, or any combination of suitable clauses, wherein the DMR associated gene category is a protease gene category.
346. The kit of clause 335, any other suitable clause, or any combination of suitable clauses, wherein the DMR associated gene category is a proteolysis gene category.
347. The kit of clause 335, any other suitable clause, or any combination of suitable clauses, wherein the DMR associated gene category is a translation gene category.
348. The kit of clause 335, any other suitable clause, or any combination of suitable clauses, wherein the DMR associated gene category is a binding protein gene category.
349. The kit of clause 335, any other suitable clause, or any combination of suitable clauses, wherein the DMR associated gene category is a growth factor/cytokine gene category.
350. The kit of clause 335, any other suitable clause, or any combination of suitable clauses, wherein the DMR associated gene category is a cell cycle gene category.
351. The kit of clause 335, any other suitable clause, or any combination of suitable clauses, wherein the DMR associated gene category is an immune gene category.
352. The kit of clause 335, any other suitable clause, or any combination of suitable clauses, wherein the DMR associated gene category is an apoptosis gene category.
353. The kit of clause 335, any other suitable clause, or any combination of suitable clauses, wherein the DMR associated gene category is a Golgi gene category.
354. The kit of clause 335, any other suitable clause, or any combination of suitable clauses, wherein the DMR associated gene category is an EST gene category.
355. The kit of clause 335, any other suitable clause, or any combination of suitable clauses, wherein the DMR associated gene category is an electron transport gene category.
356. The kit of clause 335, any other suitable clause, or any combination of suitable clauses, wherein the DMR associated gene category is a protein binding gene category.
357. The kit of clause 335, any other suitable clause, or any combination of suitable clauses, wherein the DMR associated gene category is a DNA repair gene category.
358. A method comprising:
   obtaining a sample of a somatic cell from a human subject;
   isolating deoxyribonucleic acid (DNA) from the sample, thereby obtaining isolated DNA;
   fragmenting the DNA;
   isolating fragmented methylated DNA;
   detecting and determining a methylation level of a differential DNA methylation region (DMR) comprised in the isolated fragmented methylated DNA; and
   comparing the methylation level of the DMR to a reference methylation level of a corresponding reference DMR;
   wherein the comparing comprises comparing employing a computer comprising a computer processor and computer readable memory comprising computer readable instructions contained thereon,
   wherein the detecting and determining comprises i) amplifying the isolated fragmented methylated DNA, ii) sequencing the isolated fragmented methylated DNA, an amplicon thereof, or both, or iii) employing an array, or iv) any combination thereof,
   wherein a plurality of distinct DMRs are detected and compared, wherein the plurality comprises at least 10 distinct DMRs.
359. The method of clause 358, any other suitable clause, or any combination of suitable clauses, wherein the fragmenting is performed via sonication.
360. The method of clause 358, any other suitable clause, or any combination of suitable clauses, wherein the isolating the fragmented methylated DNA comprises methylated DNA immunoprecipitation (MeDIP).
361. The method of clause 358, any other suitable clause, or any combination of suitable clauses, wherein the somatic cell comprises a buccal cell.
362. The method of clause 358, any other suitable clause, or any combination of suitable clauses, wherein the somatic cell consists essentially of a buccal cell.
363. The method of clause 358, any other suitable clause, or any combination of suitable clauses, wherein the somatic cell consists of a buccal cell.
364. The method of clause 358, any other suitable clause, or any combination of suitable clauses, wherein the somatic cell comprises a blood monocyte cell.
365. The method of clause 364, any other suitable clause, or any combination of suitable clauses, wherein the blood monocyte cell is a purified blood monocyte cell.
366. The method of clause 358, any other suitable clause, or any combination of suitable clauses, wherein the somatic cell consists essentially of a blood monocyte cell.
367. The method of clause 366, any other suitable clause, or any combination of suitable clauses, wherein the blood monocyte cell is a purified blood monocyte cell.
368. The method of clause 358, any other suitable clause, or any combination of suitable clauses, wherein the somatic cell consists of a blood monocyte cell.
369. The method of clause 368, any other suitable clause, or any combination of suitable clauses, wherein the blood monocyte cell is a purified blood monocyte cell.
370. The method of clause 358, any other suitable clause, or any combination of suitable clauses, wherein the somatic cell comprises a muscle cell, a blood cell, a skin cell, a nerve cell, or any combination thereof.
371. The method of clause 358, any other suitable clause, or any combination of suitable clauses, wherein the somatic cell comprises an immune cell.
372. The method of clause 358, any other suitable clause, or any combination of suitable clauses, wherein the somatic cell comprises a muscle cell.
373. The method of clause 358, any other suitable clause, or any combination of suitable clauses, wherein the somatic cell comprises a blood cell.
374. The method of clause 358, any other suitable clause, or any combination of suitable clauses, wherein the somatic cell comprises a skin cell.
375. The method of clause 358, any other suitable clause, or any combination of suitable clauses, wherein the somatic cell comprises a nerve cell.
376. The method of clause 358, any other suitable clause, or any combination of suitable clauses, wherein the plurality of distinct DMRs are selected from the DMRs in Table 1, the DMRs in Table 2, the DMRs in Table 3, the DMRs in Table 4, and any combination thereof.
377. The method of clause 358, any other suitable clause, or any combination of suitable clauses, wherein the plurality of distinct DMRs are selected from the DMRs in Table 1.
378. The method of clause 358, any other suitable clause, or any combination of suitable clauses, wherein the plurality of distinct DMRs are selected from the DMRs in Table 2.
379. The method of clause 358, any other suitable clause, or any combination of suitable clauses, wherein the plurality of distinct DMRs are selected from the DMRs in Table 3.
380. The method of clause 358, any other suitable clause, or any combination of suitable clauses, wherein the plurality of distinct DMRs are selected from the DMRs in Table 4.
381. The method of clause 358, any other suitable clause, or any combination of suitable clauses, wherein the plurality of distinct DMRs comprises at least 10 distinct DMRs.
382. The method of clause 358, any other suitable clause, or any combination of suitable clauses, wherein the plurality of distinct DMRs comprises 2 to about 10 distinct DMRs.
383. The method of clause 358, any other suitable clause, or any combination of suitable clauses, wherein the plurality of distinct DMRs comprises 5 to about 15 distinct DMRs.
384. The method of clause 358, any other suitable clause, or any combination of suitable clauses, wherein the plurality of distinct DMRs comprises 3 to about 8 distinct DMRs.
385. The method of clause 358, any other suitable clause, or any combination of suitable clauses, wherein the plurality of distinct DMRs comprises 4 to about 12 distinct DMRs.
386. The method of clause 358, any other suitable clause, or any combination of suitable clauses, wherein the plurality of distinct DMRs comprises about 20 to about 30 distinct DMRs.
387. The method of clause 358, any other suitable clause, or any combination of suitable clauses, wherein the plurality of distinct DMRs comprises about 10 to about 1000 distinct DMRs.
388. The method of clause 358, any other suitable clause, or any combination of suitable clauses, wherein the plurality of distinct DMRs comprises about 10 to about 15 distinct DMRs.
389. The method of clause 358, any other suitable clause, or any combination of suitable clauses, wherein the plurality of distinct DMRs comprises about 10 to about 50 distinct DMRs.
390. The method of clause 358, any other suitable clause, or any combination of suitable clauses, wherein the plurality of distinct DMRs comprises about 10 to about 100 distinct DMRs.
391. The method of clause 358, any other suitable clause, or any combination of suitable clauses, wherein the plurality of distinct DMRs comprises from about 1 to about 10 distinct DMRs.
392. The method of clause 358, any other suitable clause, or any combination of suitable clauses, wherein the plurality of distinct DMRs comprises from about 5 to about 25 distinct DMRs.
393. The method of clause 358, any other suitable clause, or any combination of suitable clauses, wherein the plurality of distinct DMRs comprises from about 10 to about 50 distinct DMRs.
394. The method of clause 358, any other suitable clause, or any combination of suitable clauses, wherein the plurality of distinct DMRs comprises from about 30 to about 80 distinct DMRs.
395. The method of clause 358, any other suitable clause, or any combination of suitable clauses, wherein the plurality of distinct DMRs comprises about 50 to about 100 distinct DMRs.
396. The method of clause 358, any other suitable clause, or any combination of suitable clauses, wherein the plurality of distinct DMRs comprises about 100 to about 150 distinct DMRs.
397. The method of clause 358, any other suitable clause, or any combination of suitable clauses, wherein the plurality of distinct DMRs comprises about 150 to about 200 distinct DMRs.
398. The method of clause 358, any other suitable clause, or any combination of suitable clauses, wherein the plurality of distinct DMRs comprises about 200 to about 250 distinct DMRs.
399. The method of clause 358, any other suitable clause, or any combination of suitable clauses, wherein the plurality of distinct DMRs comprises about 250 to about 300 distinct DMRs.
400. The method of clause 358, any other suitable clause, or any combination of suitable clauses, wherein the plurality of distinct DMRs comprises about 300 to about 350 distinct DMRs.
401. The method of clause 358, any other suitable clause, or any combination of suitable clauses, wherein the plurality of distinct DMRs comprises about 350 to about 400 distinct DMRs.
402. The method of clause 358, any other suitable clause, or any combination of suitable clauses, wherein the plurality of distinct DMRs comprises about 400 to about 450 distinct DMRs.
403. The method of clause 358, any other suitable clause, or any combination of suitable clauses, wherein the plurality of distinct DMRs comprises about 450 to about 500 distinct DMRs.
404. The method of clause 358, any other suitable clause, or any combination of suitable clauses, wherein the plurality of distinct DMRs comprises about 500 to about 550 distinct DMRs.
405. The method of clause 358, any other suitable clause, or any combination of suitable clauses, wherein the plurality of distinct DMRs comprises about 550 to about 600 distinct DMRs.
406. The method of clause 358, any other suitable clause, or any combination of suitable clauses, wherein the plurality of distinct DMRs comprises about 600 to about 650 distinct DMRs.
407. The method of clause 358, any other suitable clause, or any combination of suitable clauses, wherein the plurality of distinct DMRs comprises about 650 to about 700 distinct DMRs.
408. The method of clause 358, any other suitable clause, or any combination of suitable clauses, wherein the plurality of distinct DMRs comprises about 700 to about 750 distinct DMRs.
409. The method of clause 358, any other suitable clause, or any combination of suitable clauses, wherein the plurality of distinct DMRs comprises about 750 to about 800 distinct DMRs.
410. The method of clause 358, any other suitable clause, or any combination of suitable clauses, wherein the plurality of distinct DMRs comprises about 800 to about 850 distinct DMRs.
411. The method of clause 358, any other suitable clause, or any combination of suitable clauses, wherein the plurality of distinct DMRs comprises about 850 to about 900 distinct DMRs.
412. The method of clause 358, any other suitable clause, or any combination of suitable clauses, wherein the plurality of distinct DMRs comprises about 900 to about 950 distinct DMRs.
413. The method of clause 358, any other suitable clause, or any combination of suitable clauses, wherein the plurality of distinct DMRs comprises about 950 to about 1000 distinct DMRs.
414. The method of clause 358, any other suitable clause, or any combination of suitable clauses, wherein the plurality of distinct DMRs demonstrate an increase in DNA methylation.
415. The method of clause 358, any other suitable clause, or any combination of suitable clauses, wherein the plurality of distinct DMRs demonstrate a decrease in DNA methylation.
416. The method of clause 358, any other suitable clause, or any combination of suitable clauses, wherein the plurality of distinct DMRs are of a DMR associated gene category.
417. The method of clause 416, any other suitable clause, or any combination of suitable clauses, wherein the DMR associated gene category is a signaling gene category.
418. The method of clause 416, any other suitable clause, or any combination of suitable clauses, wherein the DMR associated gene category is a transcription gene category.
419. The method of clause 416, any other suitable clause, or any combination of suitable clauses, wherein the DMR associated gene category is a metabolism gene category.
420. The method of clause 416, any other suitable clause, or any combination of suitable clauses, wherein the DMR associated gene category is a receptor gene category.
421. The method of clause 416, any other suitable clause, or any combination of suitable clauses, wherein the DMR associated gene category is a cytoskeleton gene category.
422. The method of clause 416, any other suitable clause, or any combination of suitable clauses, wherein the DMR associated gene category is a development gene category.
423. The method of clause 416, any other suitable clause, or any combination of suitable clauses, wherein the DMR associated gene category is a transport gene category.
424. The method of clause 416, any other suitable clause, or any combination of suitable clauses, wherein the DMR associated gene category is an extracellular matrix gene category.
425. The method of clause 416, any other suitable clause, or any combination of suitable clauses, wherein the DMR associated gene category is an epigenetic gene category.
426. The method of clause 416, any other suitable clause, or any combination of suitable clauses, wherein the DMR associated gene category is a protease gene category.
427. The method of clause 416, any other suitable clause, or any combination of suitable clauses, wherein the DMR associated gene category is a proteolysis gene category.
428. The method of clause 416, any other suitable clause, or any combination of suitable clauses, wherein the DMR associated gene category is a translation gene category.
429. The method of clause 416, any other suitable clause, or any combination of suitable clauses, wherein the DMR associated gene category is a binding protein gene category.
430. The method of clause 416, any other suitable clause, or any combination of suitable clauses, wherein the DMR associated gene category is a growth factor/cytokine gene category.
431. The method of clause 416, any other suitable clause, or any combination of suitable clauses, wherein the DMR associated gene category is a cell cycle gene category.
432. The method of clause 416, any other suitable clause, or any combination of suitable clauses, wherein the DMR associated gene category is an immune gene category.
433. The method of clause 416, any other suitable clause, or any combination of suitable clauses, wherein the DMR associated gene category is an apoptosis gene category.
434. The method of clause 416, any other suitable clause, or any combination of suitable clauses, wherein the DMR associated gene category is a Golgi gene category.
435. The method of clause 416, any other suitable clause, or any combination of suitable clauses, wherein the DMR associated gene category is an EST gene category.
436. The method of clause 416, any other suitable clause, or any combination of suitable clauses, wherein the DMR associated gene category is an electron transport gene category.
437. The method of clause 416, any other suitable clause, or any combination of suitable clauses, wherein the DMR associated gene category is a protein binding gene category.
438. The method of clause 416, any other suitable clause, or any combination of suitable clauses, wherein the DMR associated gene category is a DNA repair gene category.
439. The method of clause 358, any other suitable clause, or any combination of suitable clauses, comprising sequencing, wherein the sequencing comprises sequencing by synthesis, ion semiconductor sequencing, single molecule real time sequencing, nanopore sequencing, next-generation sequencing, or any combination thereof.
440. The method of clause 358, any other suitable clause, or any combination of suitable clauses, wherein the detected DMRs comprise DMRs from at least about: 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about: 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.
441. The method of clause 358, any other suitable clause, or any combination of suitable clauses, wherein the DMRs that are determined and compared, individually, range from about 100 to about 17000 adjacent nucleotides.
442. The method of clause 358, any other suitable clause, or any combination of suitable clauses, wherein each DMR in a plurality of the DMRs that are determined and compared independently comprise a CpG density of about 0.1 to about 11 CpG per 100 nucleotides.
443. The method of clause 442, any other suitable clause, or any combination of suitable clauses, wherein each DMR in a plurality of the DMRs that are determined and compared independently comprise a CpG density of about 1 to about 10 CpG per 100 nucleotides.
444. The method of clause 442, any other suitable clause, or any combination of suitable clauses, wherein each DMR in a plurality of the DMRs that are determined and compared independently comprise a CpG density of less than about 11 CpG per 100 nucleotides.
445. The method of clause 442, any other suitable clause, or any combination of suitable clauses, wherein each DMR in a plurality of the DMRs that are determined and compared independently comprise a CpG density of less than about 5 CpG per 100 nucleotides.
446. The method of clause 442, any other suitable clause, or any combination of suitable clauses, wherein each DMR in a plurality of the DMRs that are determined and compared independently comprise a CpG density of less than about 3 CpG per 100 nucleotides.
447. The method of clause 442, any other suitable clause, or any combination of suitable clauses, wherein each DMR in a plurality of the DMRs that are determined and compared independently comprise a CpG density of between 1 CpG and 3 CpG per 100 nucleotides.
448. The method of clause 358, any other suitable clause, or any combination of suitable clauses, wherein at least about 10, 20, 30, 40, 50, 60, 70, 80, or 90 percent of the DMRs that are determined and compared are hypermethylated when compared, individually, to individual reference methylation levels of corresponding individual reference DMRs.
449. The method of clause 358, any other suitable clause, or any combination of suitable clauses, wherein at least about 30, 40, or 50 percent of the DMRs that are determined and compared are hypomethylated when compared, individually, to individual reference methylation levels of corresponding individual reference DMRs.
450. The method of clause 358, any other suitable clause, or any combination of suitable clauses, wherein all of the DMRs that are determined and compared are hypomethylated when compared, individually, to individual reference methylation levels of corresponding individual reference DMRs.
451. The method of clause 358, any other suitable clause, or any combination of suitable clauses, wherein all of the DMRs that are determined and compared are hypermethylated when compared, individually, to individual reference methylation levels of corresponding individual reference DMRs.
452. The method of clause 358, any other suitable clause, or any combination of suitable clauses, wherein the human subject is a male subject.
453. The method of clause 358, any other suitable clause, or any combination of suitable clauses, wherein the human subject is a female subject.
454. The method of clause 358, any other suitable clause, or any combination of suitable clauses, wherein the human subject is a female infant.
455. The method of clause 358, any other suitable clause, or any combination of suitable clauses, wherein the human subject is a male infant.
456. The method of clause 358, any other suitable clause, or any combination of suitable clauses, wherein the method further comprises determining a risk of the human subject having preterm birth, wherein the determining is performed with a computer comprising a computer processor and computer readable memory comprising computer readable instructions.
457. The method of clause 358, any other suitable clause, or any combination of suitable clauses, further comprising performing an additional analysis using a computer.
458. The method of clause 457, any other suitable clause, or any combination of suitable clauses, wherein the additional analysis comprises a principle component analysis (PCA), a dendrogram analysis, a machine learning analysis, or any combination thereof.
459. The method of clause 457, any other suitable clause, or any combination of suitable clauses, wherein the additional analysis generates data points, and wherein the data points in the additional analysis are grouped into two spatially distinct categories comprising a first category and a second category.
460. The method of clause 459, any other suitable clause, or any combination of suitable clauses, wherein the first category indicates the human subject is at increased risk of preterm birth and wherein the second category indicates the human subject is not at increased risk of preterm birth.
461. The method of clause 358, any other suitable clause, or any combination of suitable clauses, further comprising treating the human subject.
462. The method of clause 461, any other suitable clause, or any combination of suitable clauses, wherein the treating comprises administering clinical management for prevention of preterm birth.
463. The method of clause 462, any other suitable clause, or any combination of suitable clauses, wherein the clinical management comprises enhanced monitoring of pregnancy.
464. The method of clause 462, any other suitable clause, or any combination of suitable clauses, wherein the clinical management comprises application of protective uterine monitoring.
465. The method of clause 462, any other suitable clause, or any combination of suitable clauses, wherein the clinical management comprises hospital admission of the human subject.
466. The method of clause 462, any other suitable clause, or any combination of suitable clauses, wherein the clinical management comprises operative delivery performed on the human subject.
467. The method of clause 461, any other suitable clause, or any combination of suitable clauses, wherein the treating comprises administering a medication for preventative treatment of preterm birth.
468. The method of clause 467, any other suitable clause, or any combination of suitable clauses, wherein the medication is a prenatal steroid.
469. The method of clause 467, any other suitable clause, or any combination of suitable clauses, wherein the medication is nitroglycerine.
470. The method of clause 467, any other suitable clause, or any combination of suitable clauses, wherein the medication is a tocolytic.
471. The method of clause 470, any other suitable clause, or any combination of suitable clauses, wherein the tocolytic is an oxytocin receptor (OTR) antagonist.
472. The method of clause 470, any other suitable clause, or any combination of suitable clauses, wherein the tocolytic is selected from the group consisting of a β2 agonist, a calcium channel blocker, a nonsteroidal anti-inflammatory drug (NSAID), magnesium sulfate, and any combination thereof.
473. The method of clause 470, any other suitable clause, or any combination of suitable clauses, wherein the tocolytic is a beta-adrenergic receptor agonist.
474. The method of clause 473, any other suitable clause, or any combination of suitable clauses, wherein the beta-adrenergic receptor agonist is selected from the group consisting of terbutaline, hexoprenaline, ritodrine, a salt of any of these, or any combination thereof.
475. The method of clause 470, any other suitable clause, or any combination of suitable clauses, wherein the tocolytic is a calcium channel blocker.
476. The method of clause 475, any other suitable clause, or any combination of suitable clauses, wherein the calcium channel blocker is nifedipine or a salt thereof.
477. The method of clause 470, any other suitable clause, or any combination of suitable clauses, wherein the tocolytic is an NSAID.
478. The method of clause 477, any other suitable clause, or any combination of suitable clauses, wherein the NSAID is indomethacin or a salt thereof.
479. The method of clause 470, any other suitable clause, or any combination of suitable clauses, wherein the tocolytic is magnesium sulfate.
480. The method of clause 470, any other suitable clause, or any combination of suitable clauses, wherein the tocolytic is an oxytocin inhibitor.
481. The method of clause 480, any other suitable clause, or any combination of suitable clauses, wherein the oxytocin inhibitor is atosiban, retosiban, a salt of any of these, or any combination thereof.
482. The method of clause 470, any other suitable clause, or any combination of suitable clauses, wherein the tocolytic is terbutaline.
483. The method of clause 470, any other suitable clause, or any combination of suitable clauses, wherein the tocolytic is ritodrine.
484. The method of clause 470, any other suitable clause, or any combination of suitable clauses, wherein the tocolytic is fenoterol.
485. The method of clause 470, any other suitable clause, or any combination of suitable clauses, wherein the tocolytic is albuterol.
486. The method of clause 470, any other suitable clause, or any combination of suitable clauses, wherein the tocolytic is hexoprenaline.
487. The method of clause 470, any other suitable clause, or any combination of suitable clauses, wherein the tocolytic is nifedipine.
488. The method of clause 470, any other suitable clause, or any combination of suitable clauses, wherein the tocolytic is atosiban.
489. The method of clause 470, any other suitable clause, or any combination of suitable clauses, wherein the tocolytic is indomethacin.
490. The method of clause 470, any other suitable clause, or any combination of suitable clauses, wherein the tocolytic is sulindac.
491. The method of clause 358, any other suitable clause, or any combination of suitable clauses, further comprising transmitting data, a result, or both, via an electronic communication medium.
492. A method comprising:
   obtaining a sample of a somatic cell from a human subject;
   isolating deoxyribonucleic acid (DNA) from the sample, thereby obtaining isolated DNA;
   fragmenting the DNA;
   isolating fragmented methylated DNA;
   detecting and determining a methylation level of a differential DNA methylation region (DMR) comprised in the isolated fragmented methylated DNA; and
   comparing the methylation level of the DMR to a reference methylation level of a corresponding reference DMR;
   wherein the comparing comprises comparing employing a computer comprising a computer processor and computer readable memory comprising computer readable instructions contained thereon,
   wherein the detecting and determining comprises i) amplifying the isolated fragmented methylated DNA, ii) sequencing the isolated fragmented methylated DNA, an amplicon thereof, or both, or iii) employing an array, or iv) any combination thereof; and
   wherein a plurality of determined DMRs are sufficient to determine, from a process comprising the comparing and employing a computer, whether the human subject has or is at increased risk of preterm birth.
493. The method of clause 492, any other suitable clause, or any combination of suitable clauses, wherein the fragmenting is performed via sonication.
494. The method of clause 492, any other suitable clause, or any combination of suitable clauses, wherein the isolating the fragmented methylated DNA comprises methylated DNA immunoprecipitation (MeDIP).
495. The method of clause 492, any other suitable clause, or any combination of suitable clauses, wherein the somatic cell comprises a buccal cell.
496. The method of clause 492, any other suitable clause, or any combination of suitable clauses, wherein the somatic cell consists essentially of a buccal cell.
497. The method of clause 492, any other suitable clause, or any combination of suitable clauses, wherein the somatic cell consists of a buccal cell.
498. The method of clause 492, any other suitable clause, or any combination of suitable clauses, wherein the somatic cell comprises a blood monocyte cell.
499. The method of clause 498, any other suitable clause, or any combination of suitable clauses, wherein the blood monocyte cell is a purified blood monocyte cell.
500. The method of clause 492, any other suitable clause, or any combination of suitable clauses, wherein the somatic cell consists essentially of a blood monocyte cell.
501. The method of clause 500, any other suitable clause, or any combination of suitable clauses, wherein the blood monocyte cell is a purified blood monocyte cell.
502. The method of clause 492, any other suitable clause, or any combination of suitable clauses, wherein the somatic cell consists of a blood monocyte cell.
503. The method of clause 502, any other suitable clause, or any combination of suitable clauses, wherein the blood monocyte cell is a purified blood monocyte cell.
504. The method of clause 492, any other suitable clause, or any combination of suitable clauses, wherein the somatic cell comprises a muscle cell, a blood cell, a skin cell, a nerve cell, or any combination thereof.
505. The method of clause 492, any other suitable clause, or any combination of suitable clauses, wherein the somatic cell comprises an immune cell.
506. The method of clause 492, any other suitable clause, or any combination of suitable clauses, wherein the somatic cell comprises a muscle cell.
507. The method of clause 492, any other suitable clause, or any combination of suitable clauses, wherein the somatic cell comprises a blood cell.
508. The method of clause 492, any other suitable clause, or any combination of suitable clauses, wherein the somatic cell comprises a skin cell.
509. The method of clause 492, any other suitable clause, or any combination of suitable clauses, wherein the somatic cell comprises a nerve cell.
510. The method of clause 492, any other suitable clause, or any combination of suitable clauses, wherein the plurality of determined DMRs are selected from the DMRs in Table 1, the DMRs in Table 2, the DMRs in Table 3, the DMRs in Table 4, and any combination thereof.
511. The method of clause 492, any other suitable clause, or any combination of suitable clauses, wherein the plurality of determined DMRs are selected from the DMRs in Table 1.
512. The method of clause 492, any other suitable clause, or any combination of suitable clauses, wherein the plurality of determined DMRs are selected from the DMRs in Table 2.
513. The method of clause 492, any other suitable clause, or any combination of suitable clauses, wherein the plurality of determined DMRs are selected from the DMRs in Table 3.
514. The method of clause 492, any other suitable clause, or any combination of suitable clauses, wherein the plurality of determined DMRs are selected from the DMRs in Table 4.
515. The method of clause 492, any other suitable clause, or any combination of suitable clauses, wherein the plurality of determined DMRs comprises about 1 to about 10 DMRs.
516. The method of clause 492, any other suitable clause, or any combination of suitable clauses, wherein the plurality of determined DMRs comprises about 5 to about 25 DMRs.
517. The method of clause 492, any other suitable clause, or any combination of suitable clauses, wherein the plurality of determined DMRs comprises about 10 to about 50 DMRs.
518. The method of clause 492, any other suitable clause, or any combination of suitable clauses, wherein the plurality of determined DMRs comprises about 30 to about 80 DMRs.
519. The method of clause 492, any other suitable clause, or any combination of suitable clauses, wherein the plurality of determined DMRs comprises about 50 to about 100 DMRs.
520. The method of clause 492, any other suitable clause, or any combination of suitable clauses, wherein the plurality of determined DMRs comprises about 100 to about 150 DMRs.
521. The method of clause 492, any other suitable clause, or any combination of suitable clauses, wherein the plurality of determined DMRs comprises about 150 to about 200 DMRs.
522. The method of clause 492, any other suitable clause, or any combination of suitable clauses, wherein the plurality of determined DMRs comprises about 200 to about 250 DMRs.
523. The method of clause 492, any other suitable clause, or any combination of suitable clauses, wherein the plurality of determined DMRs comprises about 250 to about 300 DMRs.
524. The method of clause 492, any other suitable clause, or any combination of suitable clauses, wherein the plurality of determined DMRs comprises about 300 to about 350 DMRs.
525. The method of clause 492, any other suitable clause, or any combination of suitable clauses, wherein the plurality of determined DMRs comprises about 350 to about 400 DMRs.
526. The method of clause 492, any other suitable clause, or any combination of suitable clauses, wherein the plurality of determined DMRs comprises about 400 to about 450 DMRs.
527. The method of clause 492, any other suitable clause, or any combination of suitable clauses, wherein the plurality of determined DMRs comprises about 450 to about 500 DMRs.
528. The method of clause 492, any other suitable clause, or any combination of suitable clauses, wherein the plurality of determined DMRs comprises about 500 to about 550 DMRs.
529. The method of clause 492, any other suitable clause, or any combination of suitable clauses, wherein the plurality of determined DMRs comprises about 550 to about 600 DMRs.
530. The method of clause 492, any other suitable clause, or any combination of suitable clauses, wherein the plurality of determined DMRs comprises about 600 to about 650 DMRs.
531. The method of clause 492, any other suitable clause, or any combination of suitable clauses, wherein the plurality of determined DMRs comprises about 650 to about 700 DMRs.
532. The method of clause 492, any other suitable clause, or any combination of suitable clauses, wherein the plurality of determined DMRs comprises about 700 to about 750 DMRs.
533. The method of clause 492, any other suitable clause, or any combination of suitable clauses, wherein the plurality of determined DMRs comprises about 750 to about 800 DMRs.
534. The method of clause 492, any other suitable clause, or any combination of suitable clauses, wherein the plurality of determined DMRs comprises about 800 to about 850 DMRs.
535. The method of clause 492, any other suitable clause, or any combination of suitable clauses, wherein the plurality of determined DMRs comprises about 850 to about 900 DMRs.
536. The method of clause 492, any other suitable clause, or any combination of suitable clauses, wherein the plurality of determined DMRs comprises about 900 to about 950 DMRs.
537. The method of clause 492, any other suitable clause, or any combination of suitable clauses, wherein the plurality of determined DMRs comprises about 950 to about 1000 DMRs.
538. The method of clause 492, any other suitable clause, or any combination of suitable clauses, wherein the plurality of distinct DMRs demonstrate an increase in DNA methylation.
539. The method of clause 492, any other suitable clause, or any combination of suitable clauses, wherein the plurality of distinct DMRs demonstrate a decrease in DNA methylation.
540. The method of clause 492, any other suitable clause, or any combination of suitable clauses, wherein the plurality of distinct DMRs are of a DMR associated gene category.
541. The method of clause 540, any other suitable clause, or any combination of suitable clauses, wherein the DMR associated gene category is a signaling gene category.
542. The method of clause 540, any other suitable clause, or any combination of suitable clauses, wherein the DMR associated gene category is a transcription gene category.
543. The method of clause 540, any other suitable clause, or any combination of suitable clauses, wherein the DMR associated gene category is a metabolism gene category.
544. The method of clause 540, any other suitable clause, or any combination of suitable clauses, wherein the DMR associated gene category is a receptor gene category.
545. The method of clause 540, any other suitable clause, or any combination of suitable clauses, wherein the DMR associated gene category is a cytoskeleton gene category.
546. The method of clause 540, any other suitable clause, or any combination of suitable clauses, wherein the DMR associated gene category is a development gene category.
547. The method of clause 540, any other suitable clause, or any combination of suitable clauses, wherein the DMR associated gene category is a transport gene category.
548. The method of clause 540, any other suitable clause, or any combination of suitable clauses, wherein the DMR associated gene category is an extracellular matrix gene category.
549. The method of clause 540, any other suitable clause, or any combination of suitable clauses, wherein the DMR associated gene category is an epigenetic gene category.
550. The method of clause 540, any other suitable clause, or any combination of suitable clauses, wherein the DMR associated gene category is a protease gene category.
551. The method of clause 540, any other suitable clause, or any combination of suitable clauses, wherein the DMR associated gene category is a proteolysis gene category.
552. The method of clause 540, any other suitable clause, or any combination of suitable clauses, wherein the DMR associated gene category is a translation gene category.
553. The method of clause 540, any other suitable clause, or any combination of suitable clauses, wherein the DMR associated gene category is a binding protein gene category.
554. The method of clause 540, any other suitable clause, or any combination of suitable clauses, wherein the DMR associated gene category is a growth factor/cytokine gene category.
555. The method of clause 540, any other suitable clause, or any combination of suitable clauses, wherein the DMR associated gene category is a cell cycle gene category.
556. The method of clause 540, any other suitable clause, or any combination of suitable clauses, wherein the DMR associated gene category is an immune gene category.
557. The method of clause 540, any other suitable clause, or any combination of suitable clauses, wherein the DMR associated gene category is an apoptosis gene category.
558. The method of clause 540, any other suitable clause, or any combination of suitable clauses, wherein the DMR associated gene category is a Golgi gene category.
559. The method of clause 540, any other suitable clause, or any combination of suitable clauses, wherein the DMR associated gene category is an EST gene category.
560. The method of clause 540, any other suitable clause, or any combination of suitable clauses, wherein the DMR associated gene category is an electron transport gene category.
561. The method of clause 540, any other suitable clause, or any combination of suitable clauses, wherein the DMR associated gene category is a protein binding gene category.
562. The method of clause 540, any other suitable clause, or any combination of suitable clauses, wherein the DMR associated gene category is a DNA repair gene category.
563. The method of clause 492, any other suitable clause, or any combination of suitable clauses, wherein the human subject is a male subject.
564. The method of clause 492, any other suitable clause, or any combination of suitable clauses, wherein the human subject is a female subject.
565. The method of clause 492, any other suitable clause, or any combination of suitable clauses, wherein the human subject is a female infant.
566. The method of clause 492, any other suitable clause, or any combination of suitable clauses, wherein the human subject is a male infant.
567. The method of clause 492, any other suitable clause, or any combination of suitable clauses, further comprising treating the human subject.
568. The method of clause 567, any other suitable clause, or any combination of suitable clauses, wherein the treating comprises administering clinical management for prevention of preterm birth.
569. The method of clause 568, any other suitable clause, or any combination of suitable clauses, wherein the clinical management comprises enhanced monitoring of pregnancy.
570. The method of clause 568, any other suitable clause, or any combination of suitable clauses, wherein the clinical management comprises application of protective uterine monitoring.
571. The method of clause 568, any other suitable clause, or any combination of suitable clauses, wherein the clinical management comprises hospital admission of the human subject.
572. The method of clause 568, any other suitable clause, or any combination of suitable clauses, wherein the clinical management comprises operative delivery performed on the human subject.
573. The method of clause 567, any other suitable clause, or any combination of suitable clauses, wherein the treating comprises administering a medication for preventative treatment of preterm birth.
574. The method of clause 573, any other suitable clause, or any combination of suitable clauses, wherein the medication is a prenatal steroid.
575. The method of clause 573, any other suitable clause, or any combination of suitable clauses, wherein the medication is nitroglycerine.
576. The method of clause 573, any other suitable clause, or any combination of suitable clauses, wherein the medication is a tocolytic.
577. The method of clause 576, any other suitable clause, or any combination of suitable clauses, wherein the tocolytic is an oxytocin receptor (OTR) antagonist.
578. The method of clause 576, any other suitable clause, or any combination of suitable clauses, wherein the tocolytic is selected from the group consisting of a β2 agonist, a calcium channel blocker, a nonsteroidal anti-inflammatory drug (NSAID), magnesium sulfate, and any combination thereof.
579. The method of clause 576, any other suitable clause, or any combination of suitable clauses, wherein the tocolytic is a beta-adrenergic receptor agonist.
580. The method of clause 579, any other suitable clause, or any combination of suitable clauses, wherein the beta-adrenergic receptor agonist is selected from the group consisting of terbutaline, hexoprenaline, ritodrine, a salt of any of these, or any combination thereof.
581. The method of clause 576, any other suitable clause, or any combination of suitable clauses, wherein the tocolytic is a calcium channel blocker.
582. The method of clause 581, any other suitable clause, or any combination of suitable clauses, wherein the calcium channel blocker is nifedipine or a salt thereof.
583. The method of clause 576, any other suitable clause, or any combination of suitable clauses, wherein the tocolytic is an NSAID.
584. The method of clause 583, any other suitable clause, or any combination of suitable clauses, wherein the NSAID is indomethacin or a salt thereof.
585. The method of clause 576, any other suitable clause, or any combination of suitable clauses, wherein the tocolytic is magnesium sulfate.
586. The method of clause 576, any other suitable clause, or any combination of suitable clauses, wherein the tocolytic is an oxytocin inhibitor.
587. The method of clause 586, any other suitable clause, or any combination of suitable clauses, wherein the oxytocin inhibitor is atosiban, retosiban, a salt of any of these, or any combination thereof.
588. The method of clause 576, any other suitable clause, or any combination of suitable clauses, wherein the tocolytic is terbutaline.
589. The method of clause 576, any other suitable clause, or any combination of suitable clauses, wherein the tocolytic is ritodrine.
590. The method of clause 576, any other suitable clause, or any combination of suitable clauses, wherein the tocolytic is fenoterol.
591. The method of clause 576, any other suitable clause, or any combination of suitable clauses, wherein the tocolytic is albuterol.
592. The method of clause 576, any other suitable clause, or any combination of suitable clauses, wherein the tocolytic is hexoprenaline.
593. The method of clause 576, any other suitable clause, or any combination of suitable clauses, wherein the tocolytic is nifedipine.
594. The method of clause 576, any other suitable clause, or any combination of suitable clauses, wherein the tocolytic is atosiban.
595. The method of clause 576, any other suitable clause, or any combination of suitable clauses, wherein the tocolytic is indomethacin.
596. The method of clause 576, any other suitable clause, or any combination of suitable clauses, wherein the tocolytic is sulindac.
597. The method of clause 492, any other suitable clause, or any combination of suitable clauses, further comprising transmitting data, a result, or both, via an electronic communication medium.

### EXAMPLES

### Example 1

### Exemplary Sample Collection and Evaluation

In the instant example, exemplary samples were collected and analyzed. The methods of the instant example were utilized for data presentation in Examples 2-5.

### Clinical Sample Collection and Analysis

The samples were provided by St. Franciscan Hospital and Indiana University School of Medicine. IU Health Hospitals (Riley Hospital for Children, IUH Methodist, IUH North) and Franciscan Health located in Indianapolis, Indiana, USA. Informed consent and HIPAA authorization was obtained from all participants prior to the clinical sample collection.

For sample collection involving human participants that are minors, informed consent from a parent and/or legal guardian for study participation was obtained prior to sample collection. Buccal samples were collected from the mother, father, and child in instances where pre-term birth occurred (case), or where term birth occurred (control), within 9 days following birth. The demographic data for these subjects is presented in Tables 5-9. Buccal swabs were stored at −80° C. until use.

As shown below, Tables 5-9 present the following data. Table 5 shows clinical sample information for the Mothers and Fathers of control samples. Table 6 shows clinical sample information for the infants of control samples. Table 7 shows clinical sample information for the Mothers and Fathers of preterm birth (PTB) samples. Table 8 shows clinical sample information for the infants of PTB samples. Table 9 shows clinical demographic information for control and PTB samples.

TABLE 5
Control Clinical Sample Information - Mother and Father

| | | Mother's | Mother's | Father's | Father's |
|---|---|---|---|---|---|
| | Case or | Ethnicity | Age at | Ethnicity | Age at |
| Kit # | Control | & Race | Enrollment | & Race | Enrollment |
| 422 | Control | NH White | 31 | NH White | 30 |
| 423 | Control | NH White | 19 | NH White | 20 |
| 432 | Control | NH Black | 20 | NH Black | 20 |
| 436 | Control | NH White | 28 | NH White | 28 |
| 421 | Control | NH White | 38 | NH White | 39 |
| 437 | Control | NH White | 26 | NH White | 27 |
| 440 | Control | NH White | 33 | NH White | 37 |
| 442 | Control | NH White | 22 | NH White | 24 |
| 425 | Control | NH White | 27 | NH White | 29 |
| 424 | Control | NH White | 31 | NH White | 33 |
| 428 | Control | NH White | 23 | NH White | 41 |
| 439 | Control | NH White | 34 | NH White | 38 |
| 426 | Control | NH White | 37 | NH White | 38 |
| 430 | Control | NH White | 32 | NH White | 31 |
| 427 | Control | NH White | 27 | NH White | 29 |
| 441 | Control | NH White | 31 | NH White | 33 |
| 429 | Control | NH White | 30 | NH white | 31 |
| 438 | Control | NH Black | 29 | NH Black | 32 |
| | | & Nat | | & Nat | |
| 443 | Control | NH White | 26 | NH Black | 25 |
| 431 | Control | NH White | 29 | NH White | 34 |
| 433 | Control | NH Black | 18 | NH Black | 19 |

| | | |
|---|---|---|
| Mean ± SEM | 28.1 ± 5.5 | 30.3 ± 6.3 |

TABLE 6
Control Clinical Sample Information - Infants

| | Infant's | | | Infant | |
|---|---|---|---|---|---|
| | Ethncity | Infant | Infant | Age | |
| Kit # | & Race | Gender | Category | (weeks) | Infant Age |
| 422 | NH | male | newborn | 40 | 40 wks |
| | White | | | | |
| 423 | NH | male | newborn | 37 | 37 wks |
| | White | | | | |
| 432 | NH | female | newborn | 38 | 38 wks |
| | Black | | | | |
| 436 | NH | female | newborn | 39 | 39 wks |
| | White | | | | |
| 421 | NH | male | newborn | 38 | 38 wks |
| | White | | | | |
| 437 | NH | female | newborn | 40 | 40 wks |
| | White | | | | |
| 440 | NH | female | newborn | 38 | 38 wks |
| | White | | | | |
| 442 | NH | male | newborn | 38 | 38 wks |
| | White | | | | |
| 425 | NH | male | newborn | 41 | 41 wks |
| | White | | | | |
| 424 | NH | female | newborn | 38 | 38 wks |
| | White | | | | |
| 428 | NH | male | newborn | 39 | 39 wks |
| | White | | | | |
| 439 | NH | male | newborn | 39 | 39 wks |
| | White | | | | |
| 426 | NH | female | newborn | 39 | 39 wks |
| | White | | | | |
| 430 | NH | male | newborn | 39 | 39 wks |
| | White | | | | |
| 427 | NE | female | newborn | 37 | 37 wks |
| | White | | | | |
| 441 | NH | female | newborn | 39 | 39 wks |
| | White | | | | |
| 429 | NH | female | newborn | 39 | 39 wks |
| | White | | | | |
| 438 | NH | female | newborn | 39 | 39 wks |
| | Black & | | | | |
| | Nat | | | | |
| 443 | NH | male | newborn | 39 | 39 wks |
| | White | | | | |
| 431 | NH | male | newborn | 39 | 39 wks |
| | White | | | | |
| 433 | NH | female | newborn | 39 | 39 wks |
| | Black | | | | |

| | |
|---|---|
| Mean ± SEM | 38.7 ± 0.9 |

TABLE 7
Preterm Birth (PTB) Clinical Sample
Information - Mother and Father

| | | Mother's | Mother's | Father's | Father's |
|---|---|---|---|---|---|
| | Case or | Ethnicity | Age at | Ethnicity | Age at |
| Kit # | Control | & Race | Enrollment | & Race | Enrollment |
| 400 | PTB case | Hispanic | 31 | NH White | 41 |
| | | White | | | |
| 401 | PTB case | NH White | 29 | NH White | 30 |
| 403 | PTB case | NH Black | 21 | NH Black | 24 |
| 402 | PTB case | NH White | 34 | NH White | 29 |
| 404 | PTB case | NH White | 31 | NH White | 34 |
| 405 | PTB case | NH White | 34 | NH White | 37 |
| 406 | PTB case | NH White | 31 | NH White | 30 |
| 407 | PTB case | NH White | 25 | NH White | 29 |
| 408 | PTB case | NH White | 29 | NH | 34 |
| | | & Nat | | White & | |
| | | | | Nat | |
| 411 | PTB case | NH White | 34 | NH White | 38 |
| 409 | PTB case | NH White | 29 | NH White | 29 |
| 414 | PTB case | NH White | 28 | NH White | 31 |
| 413 | PTB case | NH White | 27 | NH White | 25 |
| 410 | PTB case | NH White | 30 | NH White | 35 |
| 412 | PTB case | NH White | 25 | NH White | 28 |
| 416 | PTB case | NH White | 30 | NH White | 28 |
| 417 | PTB case | NH White | 26 | Hispanic | 27 |
| | | | | White | |
| 418 | PTB case | NH White | 24 | Unknown | 27 |
| | | | | White | |
| 419 | PTB case | NH Black | 27 | NH Black | 29 |

| | | |
|---|---|---|
| Mean ± SEM | 28.7 ± 3.5 | 30.8 ± 4.5 |

TABLE 8
Preterm Birth (PTB) Clinical Sample Information - Infants

| | Infant's | | | Infant | |
|---|---|---|---|---|---|
| | Ethncity | Infant | Infant | Age | |
| Kit # | & Race | Gender | Category | (weeks) | Infant Age |
| 400 | NH | male | newborn | 32 | 32 wks |
| | White | | | | |
| 401 | NH | male | newborn | 24 | 24 wks |
| | White | | | | |
| 403 | NH | female | newborn | 30 | 30 wks |
| | Black | | | | |
| 402 | NH | female | newborn | 31 | 31 wks |
| | White | | | | |
| 404 | NH | male | newborn | 30 | 30 wks |
| | White | | | | |
| 405 | NH | female | newborn | 30 | 30 wks |
| | White | | | | |
| 406 | NH | female | newborn | 24 | 24 wks |
| | White | | | | |
| 407 | NH | male | newborn | 32 | 32 wks |
| | White | | | | |
| 408 | NH | male | newborn | 32 | 32 wks |
| | White & | | | | |
| | Nat | | | | |
| 411 | NH | female | newborn | 31 | 31 wks |
| | White | | | | |
| 409 | NH | male | newborn | 34 | 34 wks |
| | White | | | | |
| 414 | NH | Male | newborn | 23 | 23 wks |
| | White | | | | |
| 413 | NH | female | newborn | 30 | 30 wks |
| | White | | | | |
| 410 | NH | male | newborn | 32 | 32 wks |
| | White | | | | |
| 412 | NH | female | newborn | 33 | 33 wks |
| | White | | | | |
| 416 | NH | female | newborn | 30 | 30 wks |
| | White | | | | |
| 417 | Hispanic | male | newborn | 34 | 34 wks |
| | White | | | | |
| 418 | NH | female | newborn | 29 | 29 wks |
| | White | | | | |
| 419 | NH | female | newborn | 33 | 33 wks |
| | Black | | | | |

| | | |
|---|---|---|
| Mean ± SEM | 28.7 ± 3.5 | 30.8 ± 4.5 |

TABLE 9
Clinical Demographics

| | | Term Birth | All | |
|---|---|---|---|---|
| | Preterm Birth | Controls | Births | |
| Variables | Cases (n = 19) | (n = 21) | (n = 40) | p value |
| Parental Characteristics | | | | |
| Maternal Age (years), mean (SD) | 28.7 (3.54) | 28.1 (5.51) | 28.4 (4.63) | 0.694 |
| Paternal Age (years), mean (SD) | 30.8 (4.54) | 30.4 (6.38) | 30.6 (5.51) | 0.818 |
| Maternal BMI at Delivery (kg/m²), n (%) | 33.0 (17.66) | 32.9 (7.82) | 33.0 (13.24) | 0.976 |
| Maternal BMI Categories, n (%) | | | | |
| Normal weight (18.5-24.9) | 6 (31.6%) | 3 (14.3%) | 9 (22.5%) | 0.425 |
| Overweight (25-29.9) | 5 (26.3%) | 7 (33.3%) | 12 (30.0%) | |
| Obese (30 or greater) | 7 (36.8%) | 11 (52.4%) | 18 (45.0%) | |
| Insurance, n (%) | | | | |
| Medicaid/Self-Pay | 10 (51.6%) | 8 (38.1%) | 18 (45.0%) | 0.356 |
| Private | 9 (47.4%) | 13 (61.9%) | 22 (55.0%) | |
| Complications of Pregnancy | | | | |
| Gravida, mean (min, max) | 3.6 (1, 10) | 1.7 (1, 5) | 2.6 (1, 10) | 0.014 |
| Primiparous (first pregnancy), n (%) | | | | |
| Yes | 5 (26.3% | 13 (61.9%) | 18 (45.0%) | 0.024 |
| No | 14 (73.7%) | 8 (38.1%) | 22 (55.0%) | |
| Multiparous with Prior | | | | |
| Preterm Birth, n (%) | | | | |
| Yes | 6 (31.6%) | 0 (0.0%) | 6 (15.0%) | 0.005 |
| No | 13 (68.4%) | 21 (100.0%) | 34 (85.0%) | |
| Multiparous with Prior | | | | |
| Pregnancy Loss, n (%) | | | | |
| Yes | 8 (42.1%) | 3 (14.3% | 11 (27.5%) | 0.049 |
| No | 11 (57.9% | 18 (85.7%) | 29 (72.5%) | |
| Substance Use (Tobacco, | | | | |
| Alcohol, Drug Use), n (%) | | | | |
| Current | 11 (57.9%) | 7 (33.3%) | 18 (45.0%) | 0.178 |
| Former | 2 (10.5%) | 7 (33.3%) | 9 (22.5%) | |
| None | 5 (26.3%) | 7 (33.3%) | 12 (30.0%) | |
| Metabolic Disorder (Diabetes and/ | | | | |
| or Thyroid Disease), n (%) | | | | |
| Yes | 5 (26.5%) | 4 (19.0%) | 9 (22.5%) | 0.455 |
| No | 12 (63.2%) | 17 (81.0%) | 29 (72.5%) | |
| Pre-eclampsia, n (%) | | | | |
| Yes | 5 (26.3%) | 1 (4.8% | 6 (15.0%) | 0.057 |
| No | 14 (73.7%) | 20 (95.2%) | 34 (85.0%) | |
| Obesity at End of Pregnancy, n (%) | | | | |
| Yes | 7 (36.8%) | 11 (52.4%) | 18 (45.0%) | 0.399 |
| No | 11 (57.9%) | 10 (47.6%) | 21 (52.5%) | |
| Placenta Disorders (placenta previa/ | | | | |
| accreta/percreta, placenta abruption), n (%) | | | | |
| Yes | 4 (21.1%) | 1 (4.8%) | 5 (12.5%) | 0.120 |
| No | 15 (78.9%) | 20 (95.2%) | 35 (87.5%) | |
| Cervical Insufficiency/ | | | | |
| Shortened Cervix, n (%) | | | | |
| Yes | 4 (21.1%) | 0 (0.0%) | 4 (10.0%) | 0.190 |
| No | 13 (68.4%) | 21 (100.0%) | 34 (85.0%) | |
| Maternal Infection (bacterial or viral | | | | |
| infection including UTI and STD), n (%) | | | | |
| Yes | 10 (52.6%) | 9 (42.9%) | 19 (47.5%) | 0.194 |
| No | 7 (36.8%) | 12 (57.1%) | 19 (47.5%) | |
| Neuropsychological Disorders | | | | |
| (anxiety, depression, ADHD, bipolar, | | | | |
| schizophrenia, epilepsy, migraines), n (%) | | | | |
| Yes | 12 (63.2%) | 9 (42.9%) | 21 (52.5%) | 0.240 |
| No | 5 (26.3%) | 12 (57.1%) | 17 (42.5%) | |
| Labor & Delivery Complications | | | | |
| Medical Indicated Delivery, n (%) | | | | |
| Yes | 15 (78.9%) | 8 (38.1%) | 23 (57.5%) | 0.009 |
| No | 4 (21.1%) | 13 (61.9%) | 17 (42.5%) | |
| Fetal Distress, n (%) | | | | |
| Yes | 5 (26.3%) | 1 (4.8%) | 6 (15.0%) | 0.057 |
| No | 14 (73.7%) | 20 (95.2%) | 34 (85.0%) | |
| Meconium Stained Amniotic | | | | |
| Fluid (MSAF), n (%) | | | | |
| Yes | 3 (15.8%) | 8 (38.1%) | 11 (27.5%) | 0.190 |
| No | 15 (78.9%) | 13 (61.9%) | 28 (70.0%) | |
| Infant Characteristics | | | | |
| Infant ethnicity/race, n (%) | | | | |
| Hispanic | 1 (5.3%) | 0 (0.0%) | 1 (2.5%) | 0.746 |
| Non-Hispanic White | 15 (78.9%) | 17 (81.0%) | 32 (80.0%) | |
| Non-Hispanic Black | 2 (10.5%) | 3 (14.3%) | 5 (12.5%) | |
| Non-Hispanic Other | 1 (5.3%) | 1 (4.8%) | 2 (5.0%) | |
| Infant gender, n (%) | | | | |
| Female | 10 (47.6%) | 11 (52.4%) | 21 (52.5%) | 0.987 |
| Male | 9 (52.4%) | 10 (47.6%) | 19 (47.5%) | |
| Birth Gestation (days), mean (SD) | 214 (22.68) | 274 (5.77) | 245 (34.30) | <0.001 |
| Birth Gestation (weeks), mean (min, max) | 30.2 (23, 34) | 38.8 (37, 41) | 34.7 (23, 41) | <0.001 |
| Birth Weight (grams), mean (SD) | 1568 (577.07) | 3333 (593.17) | 95 (1063.53) | <0.001 |
| Birth Weight Percentile, mean (SD) | 55.9 (24.74) | 48.1 (31.57) | 51.2 (28.45) | 0.392 |
| Fetal Growth, n (%) | | | | |
| Small for Gestational Age (SGA) | 2 (10.5%) | 1 (4.8%) | 3 (7.5%) | 0.198 |
| Appropriate for Gestational Age | 17 (89.5%) | 17 (81.0) | 34 (85.0%) | |
| (AGA) | | | | |
| Large for Gestational Age (LGA) | 0 (0.0%) | 3 (14.3) | 3 (7.5%) | |
| 5 Minute APGAR Scores, mean | 6 (2, 9) | 9 (8, 9) | 7 (2, 9) | <0.001 |
| (min, max) | | | | |
| 5 Minute APGAR Scores, n (%) | | | | |
| Less than 5 | 8 (42.9%) | 0 (0.0%) | 8 (20%) | <0.001 |
| 5 or Greater | 11 (57.9%) | 21 (100.0%) | 32 (80%) | |
| Hospital Length of Stay (days), | 52 (15, 124) | 3 (2, 4) | 27 (2, 124) | <0.001 |
| mean (min, max) | | | | |

### DNA Preparation

Frozen human buccal samples were thawed for analysis. Genomic DNA from buccal samples was prepared as follows. The buccal brush was suspended in 750 μl of cell lysis solution and 3.5 μl of Proteinase K (20 mg/ml). This suspension was incubated at 55° C. for 3 hours, then vortexed and centrifuged briefly. The lysis solution was then transferred to a new 1.5 μl microcentrifuge tube. The microcentrifuge tube with the buccal brush was centrifuged again to retain any remaining solution which was combined with the transferred lysis solution. The buccal brush was discarded and 300 μl of protein precipitation solution (Promega, A795A, Madison, WI) was added to the lysis solution. The sample was incubated on ice for 15 minutes, then centrifuged at 4° C. for 30 minutes. The supernatant was transferred to a fresh 2 mL microcentrifuge tube and 1,000 μl ice cold isopropanol was added along with 2 μL glycoblue. This suspension was mixed thoroughly and incubated at −20° C. overnight. The suspension was then centrifuged at 4° C. for 20 minutes, the supernatant was discarded, and the pellet was washed with 75% ethanol, then air-dried and resuspended in 100 μl H2O. DNA concentration was measured using the Nanodrop (Thermo Fisher, Waltham, MA).

### Methylated DNA Immunoprecipitation (MeDIP)

Methylated DNA Immunoprecipitation (MeDIP) with genomic DNA was performed as follows. Individual DNA samples (2-4 μg of total DNA) were diluted to 130 μl with 1× Tris-EDTA (TE, 10 mM Tris, 1 mM EDTA) and sonicated with the Covaris M220 using the 300 bp setting. Fragment size was verified on a 2% E-gel agarose gel. The sonicated DNA was transferred from the Covaris tube to a 1.7 ml microfuge tube, and the volume was measured.

The sonicated DNA was then diluted with TE buffer (10 mM Tris HCl, pH7.5; 1 mM EDTA) to 400 μl, heat-denatured for 10 minutes at 95° C., then immediately cooled on ice for 10 minutes. Then 100 μl of 5× IP buffer and 5 μg of antibody (monoclonal mouse anti 5-methyl cytidine; Diagenode #C15200006) were added to the denatured sonicated DNA. The DNA-antibody mixture was incubated overnight on a rotator at 4° C. The following day magnetic beads (Dynabeads M-280 Sheep anti-Mouse IgG; 11201D) were pre-washed as follows: The beads were resuspended in the vial, then the appropriate volume (50 μl per sample) was transferred to a microfuge tube. The same volume of Washing Buffer (at least 1 mL 1×PBS with 0.1% BSA and 2 mM EDTA) was added and the bead sample was resuspended. The tube was then placed into a magnetic rack for 1-2 minutes and the supernatant was discarded. The tube was removed from the magnetic rack and the beads were washed once.

The washed beads were resuspended in the same volume of 1×IP buffer (50 mM sodium phosphate pH 7.0, 700 mM NaCl, 0.25% TritonX-100) as the initial volume of beads. 50 μl of beads were added to the 500 μl of DNA-antibody mixture from the overnight incubation, then incubated for 2 hours on a rotator at 4° C. After the incubation, the bead-antibody-DNA complex was washed three times with 1×IP buffer as follows: The tube was placed into a magnetic rack for 1-2 minutes and the supernatant was discarded, then the magnetic bead antibody pellet was washed with 1×IP buffer 3 times. The washed bead antibody DNA pellet was then resuspended in 250 μl digestion buffer with 3.5 μl Proteinase K (20 mg/ml). The sample was incubated for 2-3 hours on a rotator at 55° C., then 250 μl of buffered Phenol-Chloroform-Isoamylalcohol solution was added to the sample, and the tube was vortexed for 30 seconds and then centrifuged at 14,000 rpm for 5 minutes at room temperature. The aqueous supernatant was carefully removed and transferred to a fresh microfuge tube. Then 250 μl chloroform were added to the supernatant from the previous step, vortexed for 30 seconds and centrifuged at 14,000 rpm for 5 minutes at room temperature. The aqueous supernatant was removed and transferred to a fresh microfuge tube. To the supernatant 2 μl of glycoblue (20 mg/ml), 20 μl of 5M NaCl and 500 μl ethanol were added and mixed well, then precipitated in −20° C. freezer for 1 hour to overnight. The precipitate was centrifuged at 14,000 rpm for 20 minutes at 4° C. and the supernatant was removed, while not disturbing the pellet. The pellet was washed with 500 μl cold 70% ethanol in −20° C. freezer for 15 minutes then centrifuged again at 14,000 rpm for 5 minutes at 4° C. and the supernatant was discarded. The tube was spun again briefly to collect residual ethanol to the bottom of the tube and as much liquid as possible was removed with gel loading tip. The pellet was air-dried at RT until it looked dry (about 5 minutes) then resuspended in 20 μl H2O or TE. DNA concentration was measured in Qubit (Life Technologies) with ssDNA kit (Molecular Probes Q10212).

### MeDIP-Seq Analysis

The MeDIP DNA samples (50 ng of each) were used to create libraries for next generation sequencing (NGS) using the NEBNext Ultra RNA Library Prep Kit for Illumina (San Diego, CA) starting at step 1.4 of the manufacturer's protocol to generate double stranded DNA. After this step the manufacturer's protocol was followed. Each sample received a separate index primer. NGS was performed at WSU Spokane Genomics Core using the Illumina HiSeq 2500 with a PE50 application, with a read size of approximately 50 bp and approximately 5-35 million reads per sample, and 6-7 sample libraries each were run in one lane.

### Molecular Bioinformatics and Statistics

Basic read quality was verified using information produced by the FastQC program. Reads were filtered and trimmed to remove low quality base pairs using Trimmomatic. The reads for each sample were mapped to the GRCh38 human genome using Bowtie2 with default parameter options. The mapped read files were then converted to sorted BAM files using SAMtools. To identify DMR, the reference genome was broken into 1000 bp windows. The MEDIPS R package was used to calculate differential coverage between control and exposure sample groups. The edgeR p-value was used to determine the relative difference between the two groups for each genomic window. Windows with an edgeR p-value less than 104 were considered DMRs.

The DMR edges were extended until no genomic window with an edgeR p-value less than 0.1 remained within 1000 bp of the DMR. CpG density and other information was then calculated for the DMR based on the reference genome. DMR were annotated using the NCBI provided annotations. The genes that overlapped with DMR were then input into the KEGG pathway search to identify associated pathways. The DMR associated genes were then sorted into functional groups by reducing Panther protein classifications into more general categories. All MeDIP-Seq genomic data obtained in the current study have been deposited in the NCBI public GEO database (GEO #: GSE194227).

Blinded test set analysis was performed to classify test samples into case or control groups. Samples from ten novel trios were collected to evaluate the efficacy of using the DMR sets identified as a biomarker for preterm birth. The test samples were processed identically to the samples used in the main analysis. PCA and cluster dendrogram analyses were used to search for test samples that clustered with the known samples when only DMR sites were considered. Additionally, linear discriminant analysis (LDA) and random forest (RF) classification was performed to identify which blinded samples were preterm birth, as previously described.

### Example 2

### Analysis of Buccal Cell Samples

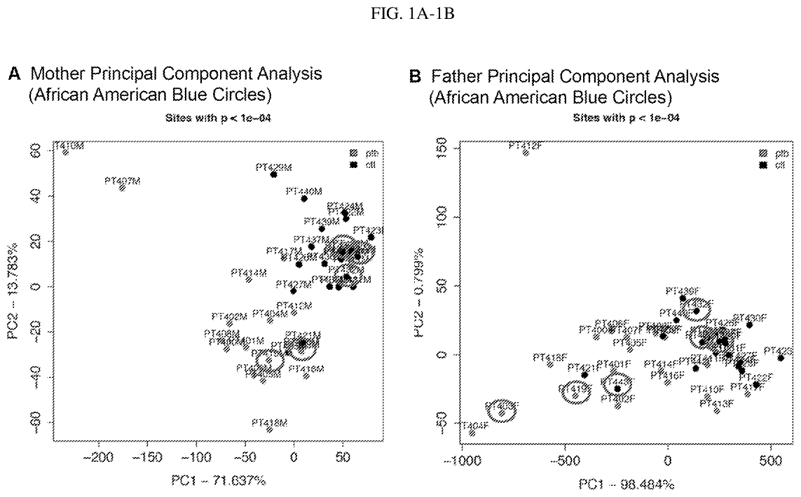

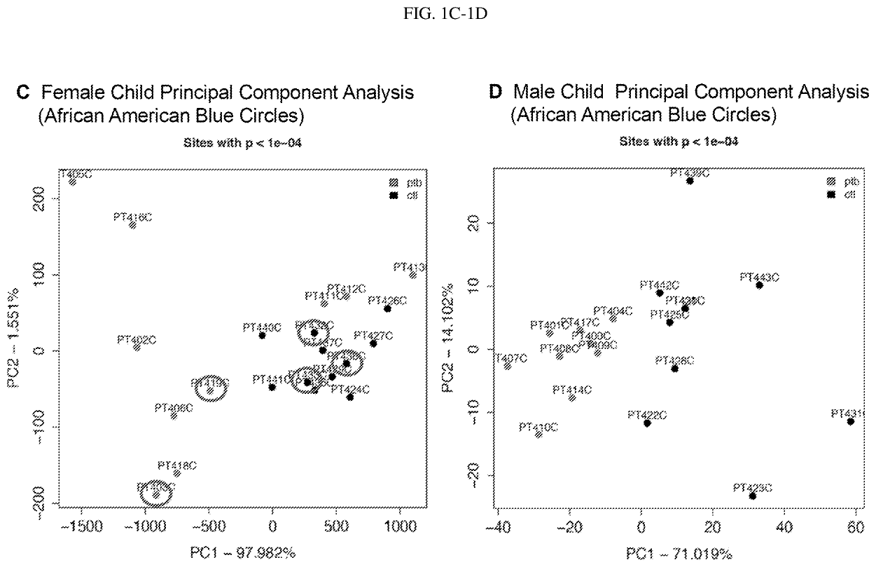

As described in Example 1, buccal cells were collected from the mother, father, and newborn child (triads) to assess epigenetic biomarkers in each group separately. The full term (FT) birth controls had 21 triad participants and the pre-term birth (PTB) cases had 19 triad participants. Although the majority were of non-Hispanic white Caucasian backgrounds, a number of triads in each population were of African American descent (Tables 5-8). The presence of the African American participants did not appear to affect the analysis and similar methylation data was observed in these samples, as assessed with a principal component analysis (PCA) and shown in FIGS. 1A-1D.

The mean maternal age was 28.1 years (controls) and 28.7 years (PTB cases) and mean paternal age 30.8 years (controls) and 30.4 years (PTB cases) with no observed statistical difference between the control or PTB case groups and with no statistical difference between the groups (Tables 5-9). The newborn gestational age at birth, mean±SD was 38.8±0.94 weeks for the control group and 30.2±3.24 weeks for the PTB case group, with statistical difference (p≤0.001) (see Tables 5-8).

Table 9 presents the clinical demographics for the populations. The preterm pregnancies were found to be significantly more likely to be multiparous and less likely to be primiparous. Therefore, without being bound by any theory, PTB occurrences appear more likely to have had one or more of the following clinical conditions: 1) to have had a previous preterm birth or pregnancy loss; 2) more likely to have preeclampsia; 3) to have a medically indicated delivery; and/or 4) to have a delivery accompanied by fetal distress and lower APGAR scores. Preterm infants naturally would have had lower birth weights, shorter gestation, and longer hospital stay. Other maternal characteristics were not significantly different between groups (e.g., maternal age, paternal age, BMI, insurance source, substance use, diabetes, thyroid placental disorders, cervical disorders, infections, neuropsychiatric disorders) (see Table 2). The PCA analysis also indicated no major outliers based on clinical parameters were detected, as shown in FIGS. 1A-1D.

### Example 3

Analysis of DMRs from Samples

DNA was isolated from the buccal cell collections and analyzed with a methylated DNA immunoprecipitation (MeDIP) procedure to obtain methylated DNA for subsequent sequencing (Seq) for an MeDIP-Seq protocol. The procedure provides a genome-wide assessment of greater than 90% of the genome, compared to approximately 50% for bisulfite sequencing or less than 1% for array analysis. Differential DNA methylation regions (DMRs) were identified by comparing the control and PTB case samples for each mother, father, or child triad. DMRs identified were obtained for each group and presented in FIG. 2A for the mother, FIG. 2B for the father, FIG. 2C for the female child, and FIG. 2D for the male child. The DMRs at various EdgeR p-value statistical thresholds are presented, and p<1e-04 was used for all subsequent data analysis, which was selected as it also provided a reasonable false discovery rate (FDR) analysis.

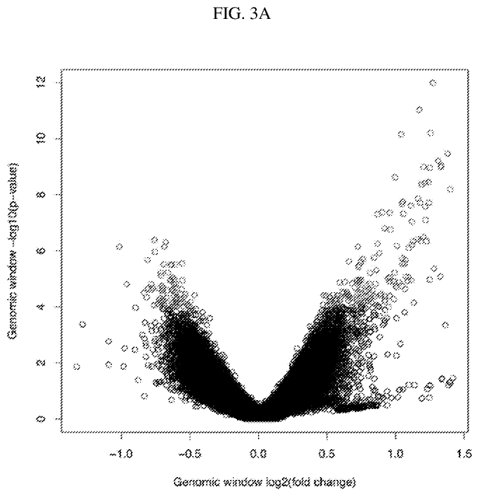

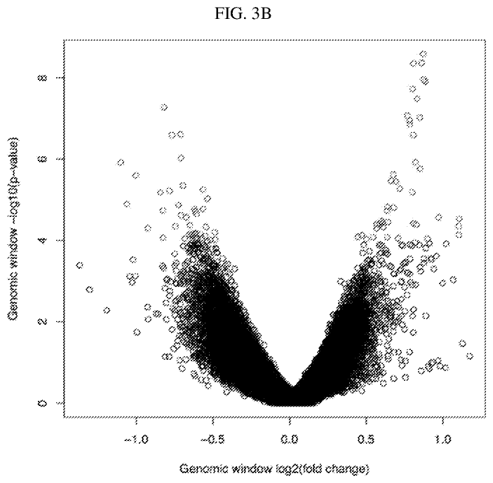

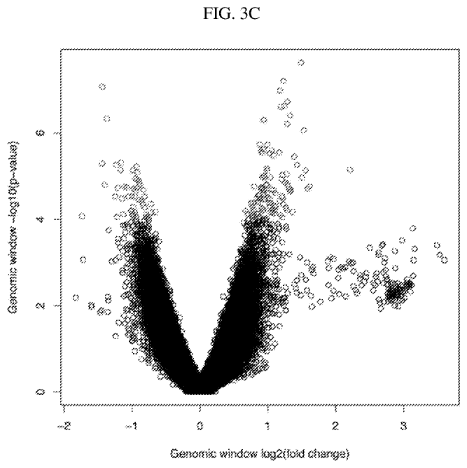

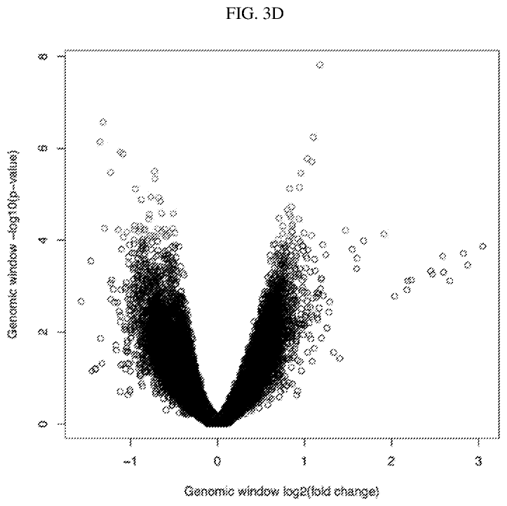

The number of adjacent DMR 1 kb windows are shown at a significance level of p<1e-04 and the majority of DMR for each group had a single 1 kb window with some higher numbers of significant adjacent windows as shown in FIGS. 2A-2D. Maternal buccal cells had 165 DMRs, paternal 73 DMRs, female child 136 DMRs, and male child 61 DMRs. The FDR p-value was less than 0.1 for 100% of the mother DMRs, 75% for the father DMRs, 50% for the female child, and 25% (i.e., 14 DMRs) for the male child. Therefore, the male child had negligible significant DMRs as shown in FIG. 2D. Approximately 50% of DMRs showed an increase (e.g., hypermethylation) and 50% a decrease (e.g., hypomethylation) in DNA methylation in each group (see FIGS. 2E and 2F and FIGS. 3A-3D).

An overlap of the DMRs demonstrated each group was primarily distinct at p<1e-04, except for the mother and female child, which shared 31 DMRs in common as shown in FIG. 4A. Further analysis of potential overlaps used an extended overlap analysis with a comparison of the p<1e-04 DMRs with the other groups at a p<0.05 threshold. This extended overlap demonstrated much higher levels of overlaps with maternal DMRs having a 49% overlap with the paternal, 58% with the female child, and 30% with the male child. Paternal DMRs had a 75% overlap with the mother, 64% with the female child, and 47% with the male child. The female child overlaps were higher and ranged from 34% to 58%, while the male child overlap ranged from 18% to 28% as shown in FIG. 4B. Therefore, preterm birth DMR were identified in the buccal cells of the mother and father, as well as in the female children following a preterm birth.

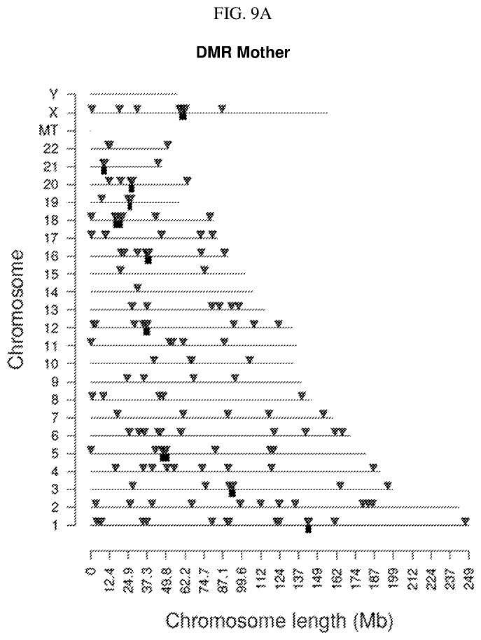

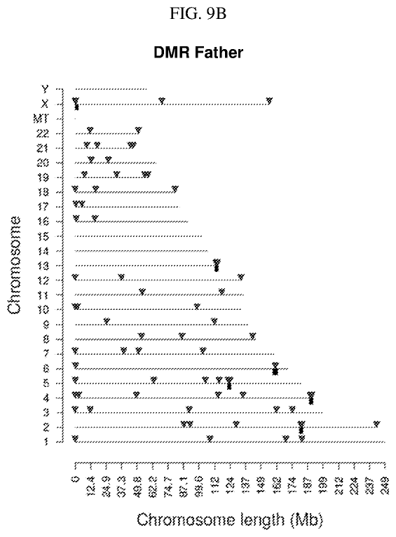

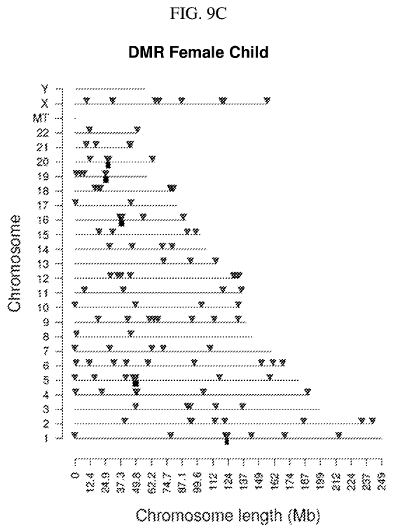

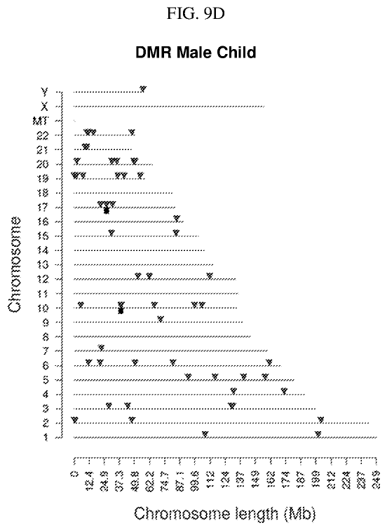

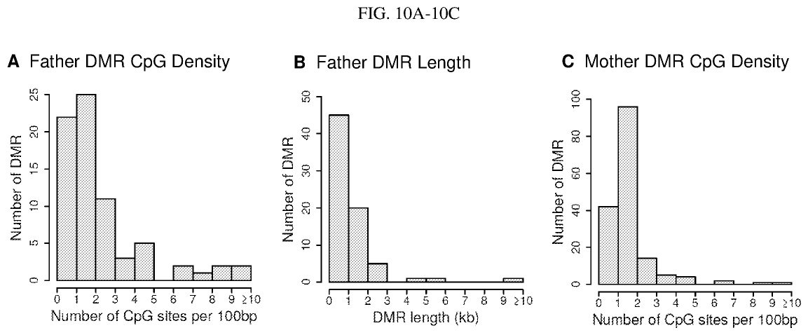

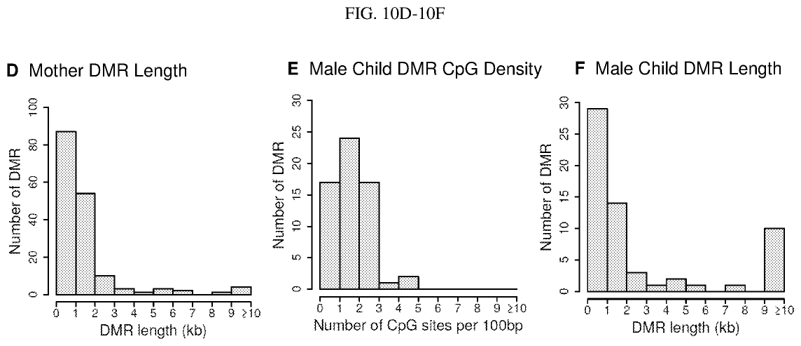

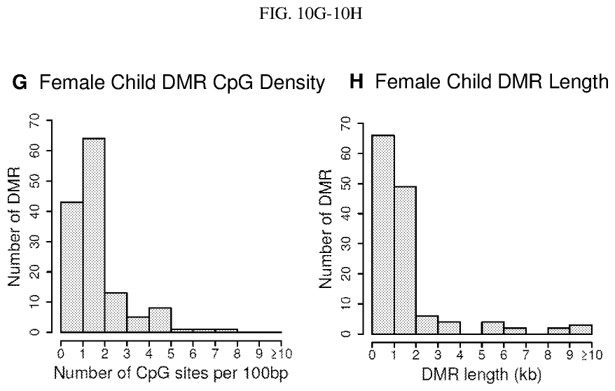

The lists of DMRs and genomic information are presented in Table 1 for the mother, Table 2 for the father, Table 3 for the female child, and Table 4 for the male child. These tables present for each group the DMR name, chromosomal location, start and stop nucleotide number, statistics information (p-value and FDR), log-fold methylation change (increase positive or decrease negative) for each DMR, gene associations (within 10 kb of gene) and functional categories for the associated genes. FIGS. 9A-9D present the chromosomal locations of the DMRs (arrowheads) for each group. The DMRs are present on most chromosomes throughout the genome. The black boxes indicate clusters of DMRs at similar regions. Although some individual DMR overlaps as a 1 kb level are observed (see FIGS. 4A-4B) no obvious gross (Mb) common chromosomal regions or sites are observed with the mother, father or female child genomes, as shown in FIGS. 9A-9D. The size of the DMRs for each group is 1 or 2 kb with a CpG density less than 3 CpG/100 bp, as shown in FIGS. 10A-10H. These regions with low CpG density are considered CpG deserts, which represents the majority (>90%) of the genome, but some DMRs are observed at higher 8-10 CpG/100 bp density associated with CpG islands.

The MeDIP procedure used in the instant example provides detection of DNA methylation in regions of lower CpG density of <5 CpG/100 bp, which corresponds to >95% of the genome. Using the genome-wide MeDIP procedure to identify DNA methylation alterations significantly increases the feasibility of finding robust epigenetic biomarkers of preterm birth.

### Example 4

### Principal Component Analysis (PCA) of DMRs

A principal component analysis (PCA) of the DMRs for the control and case comparison for each group are presented in FIGS. 1A-1D. Generally, the case and control principal component 1 and 2 separated the DMRs (see FIGS. 1A-1D). The African American samples (circled) were generally clustered with the appropriate case or control groups (see FIG. 1A). Therefore, the racial background did not appear to have major impacts. Further, the various clinical parameters in Tables 5-9 did not correlate with outliers in the PCA analysis as shown in FIGS. 1A-ID. Therefore, the DMRs identified appear to reflect PTB rather than specific pathway parameters or race.

A blinded test set of samples were collected to help validate the predictive ability of the PTB samples that were identified. Five triads for control and five triads for PTB case were collected for analysis. The test set was analyzed and the data used in dendrogram, machine learning and PCA analysis.

The accuracy for the test set of the mother was determined to be 50%, for the father was determined to be 40%, and for the female child was determined to be 60%. However, after the analyses of the unblinded samples, a very heterogenous equal mixture of moderate, very, and extreme PTB were present.

### Example 5

### Analysis of DMR Associated Genes

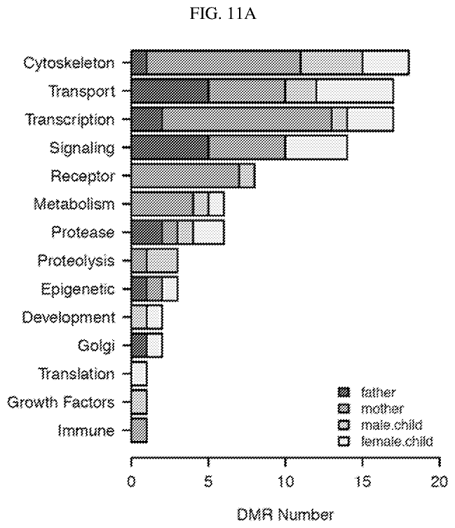

The final analysis investigated the DMR associated genes with each mother, father, and child DMR sets. The DMRs within 10 kb of a gene were considered to include proximal and distal promoter regions, as well as the gene. The DMR associated genes listed in Tables 1-4 were identified for gene functional category (see FIG. 11A). The cytoskeleton, transport, transcription, and signaling categories were prominent in each group. The DMR associated gene groups were analyzed for KEGG pathways with ≥3 genes in the pathway, and the pathways and genes presented for each group (see FIG. 11B). The mother DMR associated genes had the highest number of pathways with metabolism, synaptic vesicle cycle, and a number of signaling pathways prominent. The father had metabolism pathway, and male child no pathways. Interestingly, both the mother and female child had microRNA pathways represented (highlighted) as shown in FIG. 11B. This reflects DMRs shared between them that contain a cluster of genes and non-coding RNA, including Aopep (aminopeptidase O) and the micro-RNAs Mir 24-1, Mir 27b, Mir 23b, and Mir 3074. Therefore, an additional epigenetic mechanism altered in preterm birth appears to involve ncRNA that was common between the mother and daughter DMRs.

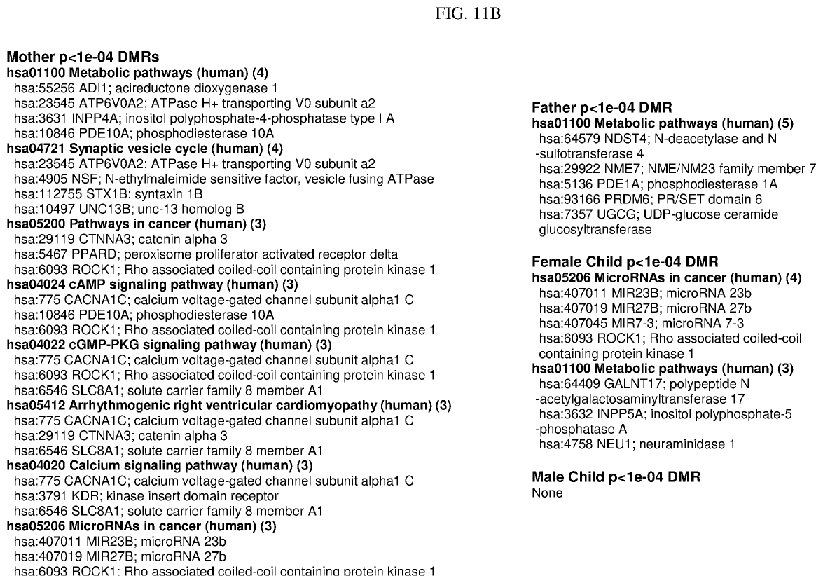

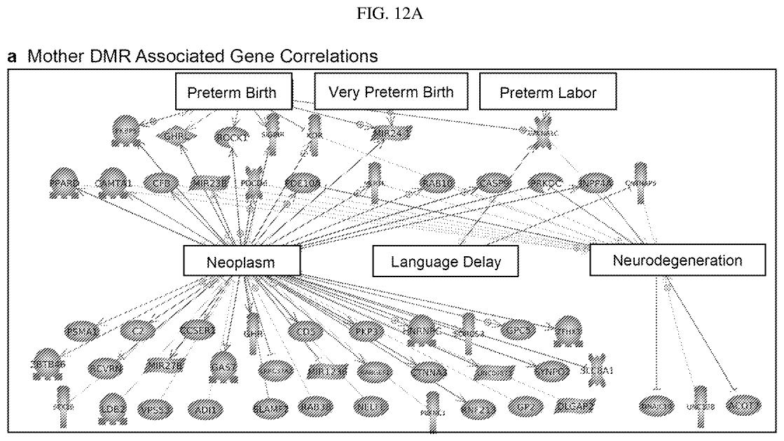

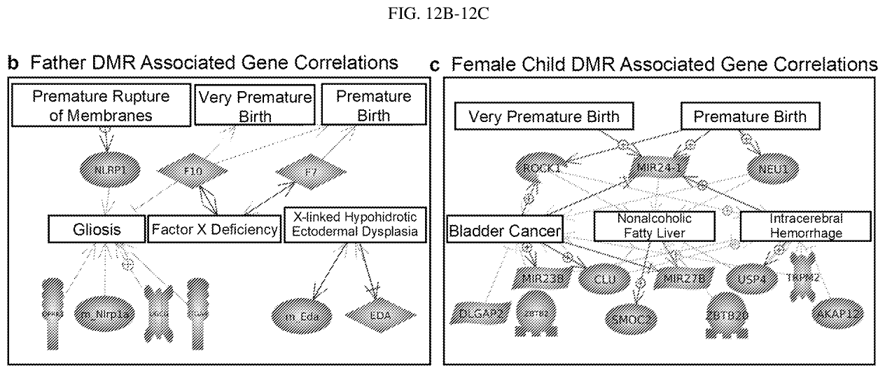

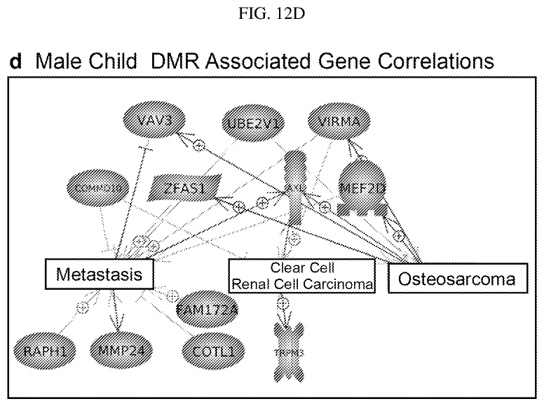

A presentation of the mother, father, and child DMR associated genes with network links, as determined by Pathway Studio (Elsevier, Inc.), are presented in FIGS. 12A-12D. For each group the three disease states most over-represented in the list of DMR-associated genes are presented. DMR associated genes with associations to premature birth are also included. The mother, father, and female child groups all had DMR-associated genes linked to preterm birth, including Rock1, Ghrl1, Fkbp5, Sigirr, Kdr, Mir24-1, Cacnalc, Neu1, Nlrp1, F7 and F10 as shown in FIGS. 12A-12D. This validates the potential PTB DMR biomarkers identified.

What is claimed is:

1. A method comprising:
obtaining a sample of a somatic cell from a human subject;
isolating deoxyribonucleic acid (DNA) from the sample, thereby obtaining isolated DNA; detecting and determining a methylation level of a differential DNA methylation region (DMR) comprised in the isolated DNA; and
comparing the methylation level of the DMR to a reference methylation level of a corresponding reference DMR;
wherein the comparing comprises employing a computer comprising a computer processor and computer readable memory comprising computer readable instructions contained thereon,
wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
wherein a plurality of distinct DMRs are detected and compared, wherein the plurality comprises at least 10 distinct DMRs.

2. The method of claim 1, wherein the somatic cell comprises a buccal cell.

3. The method of claim 1, wherein the somatic cell comprises a blood monocyte cell, and optionally wherein the blood monocyte cell is a purified blood monocyte cell.

4. The method of claim 1, wherein the plurality of distinct DMRs are selected from the DMRs in Table 1.

5. The method of claim 1, wherein the plurality of distinct DMRs are selected from the DMRs in Table 2.

6. The method of claim 1, wherein the plurality of distinct DMRs are selected from the DMRs in Table 3.

7. The method of claim 1, wherein the plurality of distinct DMRs are selected from the DMRs in Table 4.

8. The method of claim 1, wherein the plurality of distinct DMRs comprises about 10 to about 15 DMRs, about 15 to about 25 distinct DMRs, about 20 to about 40 distinct DMRs, about 30 to about 60 distinct DMRs, about 50 to about 100 distinct DMRs, or about 100 to about 150 distinct DMRs.

9. The method of claim 1, wherein the plurality of distinct DMRs comprises about 150 to about 200 distinct DMRs or about 200 to about 250 distinct DMRs.

10. The method of claim 1, comprising sequencing, wherein the sequencing comprises sequencing by synthesis, ion semiconductor sequencing, single molecule real time sequencing, nanopore sequencing, next-generation sequencing, or any combination thereof.

11. The method of claim 1, wherein the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.

12. The method of claim 1, wherein the DMRs that are determined and compared, individually, range from about 100 to about 74000 adjacent nucleotides.

13. The method of claim 1, wherein at least a plurality of the DMRs that are determined and compared comprise a CpG density of 0.1 to about 11 CpG per 100 nucleotides.

14. The method of claim 13, wherein at least a plurality of the DMRs that are determined and compared comprise a CpG density of 0.1 to about 3 CpG per 100 nucleotides.

15. The method of claim 1, wherein about 50 percent of the DMRs that are determined and compared are hypermethylated when compared, individually, to individual reference methylation levels of corresponding individual reference DMRs.

16. The method of claim 1, wherein about 50 percent of the DMRs that are determined and compared are hypomethylated when compared, individually, to individual reference methylation levels of corresponding individual reference DMRs.

17. The method of claim 1, wherein the human subject is a female subject.

18. The method of claim 1, wherein the human subject is a male subject.

19. The method of claim 1, wherein the human subject is a female infant.

20. The method of claim 1, wherein the human subject is a male infant.

21. The method of claim 1, wherein the method further comprises determining a risk of the human subject for preterm birth, wherein the determining is performed with a computer comprising a computer processor and computer readable memory comprising computer readable instructions.

22. The method of claim 1, further comprising performing an additional analysis using a computer comprising a computer processor and computer readable memory comprising computer readable instructions.

23. The method of claim 22, wherein the additional analysis comprises a principle component analysis (PCA), a dendrogram analysis, a machine learning analysis, or any combination thereof.

24. The method of claim 1, further comprising treating the human subject.

25. The method of claim 24, wherein the treating comprises administering clinical management for prevention of preterm birth.

26. The method of claim 24, wherein the treating comprises administering a medication for preventative treatment of preterm birth.

27. The method of claim 26, wherein the medication is a prenatal steroid.

28. The method of claim 26, wherein the medication is a tocolytic.

29. The method of claim 1, further comprising transmitting data, a result, or both, via an electronic communication medium.

30. A method comprising:
obtaining a sample of a somatic cell from a human subject;
isolating deoxyribonucleic acid (DNA) from the sample, thereby obtaining isolated DNA;
detecting and determining a methylation level of a differential DNA methylation region (DMR) comprised in the isolated DNA; and
comparing the methylation level of the DMR to a reference methylation level of a corresponding reference DMR;
wherein the comparing comprises employing a computer comprising a computer processor and computer readable memory comprising computer readable instructions contained thereon,
wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
wherein a plurality of determined DMRs are sufficient to determine, from a process comprising the comparing and employing a computer, whether the human subject has or is at increased risk of preterm birth.

31. The method of claim 30, wherein the somatic cell comprises a buccal cell.

32. The method of claim 30, wherein the somatic cell comprises a blood monocyte cell, and optionally wherein the blood monocyte cell is a purified blood monocyte cell.

33. The method of claim 30, wherein the plurality of determined DMRs are selected from the DMRs in Table 1.

34. The method of claim 30, wherein the plurality of determined DMRs are selected from the DMRs in Table 2.

35. The method of claim 30, wherein the plurality of determined DMRs are selected from the DMRs in Table 3.

36. The method of claim 30, wherein the plurality of determined DMRs are selected from the DMRs in Table 4.

37. The method of claim 30, wherein the plurality of determined DMRs comprises at least 10 distinct DMRs, about 10 to about 15 distinct DMRs, about 15 to about 25 distinct DMRs, about 20 to about 40 distinct DMRs, about 30 to about 60 distinct DMRs, about 50 to about 100 distinct DMRs or about 100 to about 150 distinct DMRs.

38. The method of claim 30, wherein the plurality of determined DMRs comprises about 150 to about 200 distinct DMRs or about 200 to about 250 distinct DMRs.

39. The method of claim 30, wherein the human subject is a male subject.

40. The method of claim 30, wherein the human subject is a female subject.

41. The method of claim 30, wherein the human subject is a female infant.

42. The method of claim 30, further comprising treating the human subject.

43. The method of claim 42, wherein the treating comprises administering clinical management for prevention of preterm birth.

44. The method of claim 42, wherein the treating comprises administering a medication for preventative treatment of preterm birth.

45. The method of claim 44, wherein the medication is a prenatal steroid.

46. The method of claim 44, wherein the medication is a tocolytic.

47. The method of claim 30, further comprising transmitting data, a result, or both, via an electronic communication medium.

48. A kit comprising at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence selected from the DMRs in Table 1, the DMRs in Table 2, the DMRs in Table 3, the DMRs in Table 4, and any combination thereof; and a container.

49. The kit of claim 48, wherein the distinct primers or pairs of primers each further comprise a unique barcode.

50. The kit of claim 48, wherein the distinct primers or pairs of primers are not bound to an array or a microarray.

51. The kit of claim 48, wherein the distinct primers or pairs of primers are bound to an array or a microarray.

52. The kit of claim 48, wherein the distinct primers or pairs of primers comprise DNA.

53. The kit of claim 48, wherein the distinct DMR sequences are selected from the DMRs in Table 1.

54. The kit of claim 48, wherein the distinct DMR sequences are selected from the DMRs in Table 2.

55. The kit of claim 48, wherein the distinct DMR sequences are selected from the DMRs in Table 3.

56. The kit of claim 48, wherein the distinct DMR sequences are selected from the DMRs in Table 4.

57. A kit comprising at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct probes, each distinct probe complementary to a distinct DMR sequence selected from the DMRs in Table 1, the DMRs in Table 2, the DMRs in Table 3, the DMRs in Table 4, and any combination thereof; and a container.

58. The kit of claim 57, wherein the distinct probes further comprises at least one of fluorophore, a chromophore, a barcode, or any combination thereof.

59. The kit of claim 57, wherein each probe comprises a unique fluorophore, a chromophore, barcode, or any combination thereof.

60. The kit of claim 57, wherein the distinct probes are not bound to an array or a microarray.

61. The kit of claim 57, wherein the distinct probes are bound to an array or a microarray.

62. The kit of claim 57, wherein distinct probes comprise DNA.

63. The kit of claim 57, wherein the distinct DMR sequences are selected from the DMRs in Table 1.

64. The kit of claim 57, wherein the distinct DMR sequences are selected from the DMRs in Table 2.

65. The kit of claim 57, wherein the distinct DMR sequences are selected from the DMRs in Table 3.

66. The kit of claim 57, wherein the distinct DMR sequences are selected from the DMRs in Table 4.

67. A method comprising:
obtaining a sample of a somatic cell from a human subject;
isolating deoxyribonucleic acid (DNA) from the sample, thereby obtaining isolated DNA;
fragmenting the DNA;
isolating fragmented methylated DNA;
detecting and determining a methylation level of a differential DNA methylation region (DMR) comprised in the isolated fragmented methylated DNA; and
comparing the methylation level of the DMR to a reference methylation level of a corresponding reference DMR;
wherein the comparing comprises comparing employing a computer comprising a computer processor and computer readable memory comprising computer readable instructions contained thereon,
wherein the detecting and determining comprises i) amplifying the isolated fragmented methylated DNA, ii) sequencing the isolated fragmented methylated DNA, an amplicon thereof, or both, or iii) employing an array, or iv) any combination thereof,
wherein a plurality of distinct DMRs are detected and compared, wherein the plurality comprises at least 10 distinct DMRs.

68. The method of claim 67, wherein the isolating the fragmented methylated DNA comprises methylated DNA immunoprecipitation (MeDIP).

69. The method of claim 67, wherein the somatic cells comprises a buccal cell.

70. The method of claim 67, wherein the somatic cell comprises a blood monocyte cell, wherein the blood monocyte cell is a purified blood monocyte cell.

71. The method of claim 67, wherein the plurality of distinct DMRs are selected from the DMRs in Table 1.

72. The method of claim 67, wherein the plurality of distinct DMRs are selected from the DMRs in Table 2.

73. The method of claim 67, wherein the plurality of distinct DMRs are selected from the DMRs in Table 3.

74. The method of claim 67, wherein the plurality of distinct DMRs are selected from the DMRs in Table 4.

75. A method comprising:
obtaining a sample of a somatic cell from a human subject;
isolating deoxyribonucleic acid (DNA) from the sample;
fragmenting the DNA;
isolating fragmented methylated DNA;
detecting and determining a methylation level of a differential DNA methylation region (DMR) comprised in the isolated fragmented methylated DNA; and
comparing the methylation level of the DMR to a reference methylation level of a corresponding reference DMR;
wherein the comparing comprises comparing employing a computer comprising a computer processor and computer readable memory comprising computer readable instructions contained thereon,
wherein the detecting and determining comprises i) amplifying the isolated fragmented methylated DNA, ii) sequencing the isolated fragmented methylated DNA, an amplicon thereof, or both, or iii) employing an array, or iv) any combination thereof; and
wherein a plurality of determined DMRs are sufficient to determine, from a process comprising the comparing and employing a computer, whether the human subject has or is at increased risk of preterm birth.

76. The method of claim 75, wherein the isolating the fragmented methylated DNA comprises methylated DNA immunoprecipitation (MeDIP).

77. The method of claim 75, wherein the somatic cell comprises a buccal cells.

78. The method of claim 75, wherein the somatic cell comprises a blood monocyte cell, wherein the blood monocyte cell is a purified blood monocyte cells.

79. The method of claim 75, wherein the plurality of determined DMRs are selected from the DMRs in Table 1.

80. The method of claim 75, wherein the plurality of determined DMRs are selected from the DMRs in Table 2.

81. The method of claim 75, wherein the plurality of determined DMRs are selected from the DMRs in Table 3.

82. The method of claim 75, wherein the plurality of determined DMRs are selected from the DMRs in Table 4.

83. The method of claim 75, wherein the plurality comprises at least 10 distinct DMRs.
